(19) United States
(12) Patent Application Publication
(10) Pub. No.: US 2012/0184919 A1
(43) Pub. Date: Jul. 19, 2012

(54) FAT TISSUE RE-INJECTION SYSTEM EMPLOYING A PHOTOMETRICALLY-CONTROLLED PHOTO-ACTIVATION CHAMBER INSTALLED ABOUT A TISSUE COLLECTION AND PROCESSING DEVICE MOUNTED ON A HAND-HELD TISSUE INJECTOR GUN

(76) Inventor: Robert L. Cucin, West Palm Beach, FL (US)

(21) Appl. No.: 13/315,246

(22) Filed: Dec. 8, 2011

Related U.S. Application Data
(63) Continuation of application No. 13/094,302, filed on Apr. 26, 2011.
Application No. 13/094,302 is a continuation-in-part of application No. 12/955,420, filed on Nov. 29, 2010.
Application No. 12/955,420 is a continuation-in-part of application No. 12/850,786, filed on Aug. 5, 2010.
Application No. 12/850,786 is a continuation-in-part of application No. 12/462,596, filed on Aug. 5, 2009.

(51) Int. Cl. A61M 5/31; A61M 5/315

(52) U.S. Cl. 604/218; 604/187

(57) ABSTRACT

A fat tissue re-injection system employing a photometrically-controlled photo-activation chamber installed about a sealed tissue collection and processing device mounted on a hand-held tissue injector gun.

### RELATED CASES

This Application is a Continuation of application Ser. No. 13/094,302 filed Apr. 26, 2011; which is a Continuation-in-Part (CIP) of copending application Ser. No. 12/955,420 filed Nov. 29, 2010; which is a CIP of application Ser. No. 12/850,786 filed on Aug. 5, 2010; which is a CIP of application Ser. No. 12/462,596 filed Aug. 5, 2009, and copending application Ser. No. 12/813,067 filed Jun. 10, 2010; wherein each said Application is owned by Rocin Laboratories, Inc., and incorporated herein by reference in its entirety.

### BACKGROUND OF INVENTION

1. Field of Invention

The present disclosure relates to new and improved ways of and means for collecting, processing and managing adipocyte derived stem cells (ASC) within aspirated fat tissue, for therapeutic, cosmetic and reconstructive applications.

2. Brief Description of the State of Knowledge in the Art

It is well known that fat is an ideal Mesenchymal Stem Cell (MSC) source for the following reasons: (i) the primary roles of adult stem cells are to maintain and repair the tissue in which they are found (“self-renewal”); (ii) there are two main types: Hematopoietic Stem Cells (HSCs), forming all blood cells and Mesenchymal Stem Cells (MSCs), able to differentiate into multiple cell types such as bone, fat, muscle and cartilage (“differentiation”); (iii) adipose tissue is an ideal, very rich source of adult stem cells. 5% of aspirated cells or 50 times higher concentration than in bone marrow; (iv) MSC's are robust, grow easily, are easily classified into cellular differentiated lines.

ASC's allow differentiation of all the mesenchymal stem cell lines: adipocytes; chondrocytes; osteoblasts and osteocytes; cardiomyocytes; neurons; skeletal myocytes; and endothelial cells.

Thus, stem cells are pluripotential, in that they have both the ability to replicate itself indefinitely (i.e. not to die), and to differentiate into any of the tissues enumerated above which come from mesenchymal tissues.

Liposuction popularity assures an abundant autograft source. Liposuction is one of the most common elective procedures carried out in the world. Liposuction is the most common procedure carried out for obesity treatment. Liposuction has increased 2% from 2009 to 2010. The total U.S. expenditure for liposuction in 2010 has been $585,668,787. Over 203,106 procedures were performed in 2010, with $2,884 reported as the average fee per procedure.

Currently, there are numerous markets ready for ASC lines, namely: tissue fillers; meniscular cartilage; knee and hip (for treating osteoarthritis and rheumatoid arthritis); ischemic heart damage (e.g. damaged ventricular muscle); degenerative neurologic diseases (e.g. Parkinson's Disease and Alzheimer's Disease); and degenerative muscle disease (e.g. Muscular Dystrophy).

ASC lines can be applied to numerous tissue filler treatment sites and conditions: Facial wrinkles; scars and over-treated areas; facial revoluminization. Romberg's hemifacial atrophy; microsomia; breast augmentation and breast reconstruction; buttock augmentation; and calf augmentation.

Also, there are numerous advantages to using fat and ASC-enriched autograft, than artificial tissue fillers, namely: no risk of allergy or rejection when using autografts; living tissue may give better and more sustained results; superficial mesotherapy volume restoration can be used to lessen sagging and youthen skin; the face can be “rebooted” using non-apoptotic primitive precursor adipocytes and stem cells.

Currently, Cytori's StemSource product provides 1% of 200,000 nucleated cells/ml fat aspirate. Also, Cryo-Save's Cryo-Lip product provides 5% of nucleated cells/ml. However, each of these products require exposure to collagenease and centrifuging or ultrasonic agitation, procedures which are harmful to cells and lessen cellular viability.

Thus, there is a great need in the art for a new and improved methods and apparatus for collecting, processing and managing adipocyte derived stem cell (ASC) for use in autographs and diverse forms of ASC theraphy, without the accompanying shortcomings and drawbacks of prior art techniques and methodologies.

### SUMMARY AND OBJECT OF THE INVENTION

Accordingly, a primary object of the present disclosure is to provide a new and improved method of and apparatus for photo-activating collected samples of aspirated fat tissue, including stem cells therein, to improve the proliferation, migration and adhesion thereof, during autographs transplantations, and other forms of therapeutic and/or reconstructive surgery, while avoiding the shortcomings and drawbacks of prior art methodologies.

Another object of the present disclosure is to provide a new and improved method of and system for authenticating, photo-activating, assaying, cataloguing, tracking and managing fat tissue samples, including stem cells therein, for use in autographs and other forms of therapeutic and/or reconstructive procedures.

Another object is to provide an integrated system for aspirating, collecting, concentrating, photo-activating, labeling, photo-measuring, cataloging, tracking, processing, and returning autografts of tissue and adipocyte derived stem cells (ASCs) to the patient.

Another object of the present invention is to provide an improved method of and apparatus for aspirating fat tissue from a patience using a low pressure vacuum source that minimizes cellular rupture and oils, supports gentler aspiration, and leads to higher graft survival, so that tissue can be harvested gently without heat, tissue trauma, blood loss, or surgeon's effort.

Another object of the present invention is to provide an improved method of collecting fat tissue samples including stem cells in self-contained, single-use sterile tissue collection and processing devices that employ RFID tags to identify the patient/donor source, and managing the state of collected aspirated fat tissue samples including stem cells therein, during tissue aspiration, collection, processing, and re-injection operations.

Another object of the present invention is to provide an improved method of concentrating aspirated fat tissue, including stem cells therein, while gently cleaning the same using an accompanying tumescent fluid, so that fluid, lipids, oils, contaminants and excess water passes through micro-pores formed in the walls of the syringe-like issue collection and processing devices of the present invention.

Another object of the present invention is to provide an improved method of photo-activating the cellular components of aspirated fat tissue, including stem cells therein, so as to improve graft survival, encourage cell differentiation and protein synthesis, and achieve higher levels of cellular energy.

Another object of the present invention is to provide an improved method of labeling collected samples of aspirated fat tissue, including stem cells therein, stored in syringe-like tissue collection and processing containers that have been tagged with read/write RFID tags.

Another object of the present invention is to provide an improved method of photometrically measuring, and recording, the photo-activation index (PAI) of the aspirated fat tissue sample before, during, or after photo-activation so as to expose the collected tissue sample and stem cells therein, to an adequate and not an excessive level of photo-active energy, and thus improve the vitality thereof during autografting operations.

Another object of the present invention is to provide an improved method of cataloguing, within a central networked database, information that has been recorded on the RFID tags of the tissue collection and processing devices employed in the system and across the stem cell banking network of the present invention.

Another object of the present invention is to provide an improved method of tracking collected fat tissue samples that have been harvested, processed and catalogued in a centralized system so that physician, hospitals and banks can identify the existence of, and ascertain the physical location of, such stored fat tissue samples and differentiated lines, using a Web-based database system.

Another object of the present invention is to provide an improved method of processing collected samples of aspirated fat tissue using concentration, lavage, and photo-activation operations so that harvested fat cells can be lavaged using an insulin or a growth factor enriched solution, while contained within tissue collection devices of the present invention.

Another object of the present invention is to provide an improved method of autografting of fat tissue and ASC-enriched cellular components, in an elegantly simple manner, employing manual or mechanically-assisted fat tissue reinjection devices, while completely avoiding the need for decanting, tissue transfers, autoclaving, and/or straining operations in a self-contained sterile field involving container transfers.

Another object is to provide a new and improved apparatus for photo-activating a collected sample (i.e. specimen) of fat tissue, including stems cells contained in self-contained tissue collection and processing device, by exposing the collected fat tissue sample, including stem cells therein, to low levels of photo-active light energy while contained within the tissue collection and processing device, and being photometrically-measured to determine the photo-activation index of the specimen and ensure optimized photo-activation providing the tissue sample with an improved capacity to bind free oxygen species.

Another object is to provide such apparatus in the form of a countertop supportable instrument system having a photometrically-controlled photo-activation chamber installed within the housing of the console unit, so that a sealed tissue collection and processing device can be easily inserted into the chamber of the console unit, and the fat tissue including stems cells therein undergo photometrically-controlled photo-activation treatment by low level photo-active light energy having photo-active wavelengths (e.g. 635 and 830 nanometers), emitted from arrays of visible light emitting diodes (LEDs) and/or visible laser diodes (VLDs) surrounding the tissue collection tube, while the photo-activation index (PAI) of the fat tissue sample is photometrically-measured between periodically-alternating modes of photo-activation, so as to ensure that the fat tissue sample is optimally photo-activated, and over-activation is avoided.

Another object is to provide such apparatus in the form of a countertop supportable instrument system having a photometrically-controlled photo-activation chamber installed within hand-supportable housing, so that a sealed tissue collection and processing device can be easily inserted into the chamber of the hand-supportable housing, and the fat tissue including stems cells contained therein undergo automatically-controlled photo-activation treatment by low level of photo-active light energy having photo-active wavelengths (e.g. 635 and 830 nanometers) emitted from arrays of visible light emitting diodes (LEDs) and/or visible laser diodes (VLDs) surrounding the tissue collection tube, while the photo-activation index (PAI) of the fat tissue sample is photometrically-measured between periodically-alternating modes of photo-activation, so as to ensure that the fat tissue sample is optimally photo-activated, and over-activation is avoided.

Another object is to provide such apparatus in the form of a countertop supportable instrument system having a photometrically-controlled photo-activation chamber installed about a sealed tissue collection and processing device mounted on a hand-held tissue injector gun, so that, prior to performing tissue reinjection operations, fat tissue contained in the sealed tissue collection and processing device can undergo automatically-controlled photo-activation treatment by low level of photo-active light energy having photo-active wavelengths (e.g. 635 and 830 nanometers), emitted from multiple arrays of visible light emitting diodes (LEDs) and/or visible laser diodes (VLDs) surrounding the tissue collection tube, while the photo-activation index (PAI) of the fat tissue sample is photometrically-measured between periodically-alternating modes of photo-activation, so as to ensure that the fat tissue sample is optimally photo-activated, and over-activation is avoided.

Another object is to provide such apparatus in the form of a wireless mobile hand-supportable instrument system having a photometrically-controlled photo-activation chamber installed within its hand-supportable housing, so that a sealed tissue collection and processing device can be easily inserted into the chamber of the hand-supportable housing, and the fat tissue sample including stem cells contained therein undergo automatically-controlled photo-activation treatment by low level of photo-active light energy having photo-active wavelengths (e.g. 635 and 830 nanometers) emitted from arrays of visible light emitting diodes (LEDs) and/or visible laser diodes (VLDs) surrounding the tissue collection and processing tube, while the photo-activation index (PAI) of the fat tissue sample is photometrically-measured between periodically-alternating modes of photo-activation, so as to ensure that the fat tissue sample is optimally photo-activated, and over-activation is avoided.

Another object is to provide such apparatus in the form of a hand-supportable instrument system having a photo-activation/photometric array installed within hand-supportable housing, so that in vivo fat tissue within a patient's body can undergo automatically-controlled photo-activation treatment by low level of photo-active light energy having photo-active wavelengths (e.g. 635 and 830 nanometers), emitted from arrays of visible light emitting diodes (LEDs) and/or visible laser diodes (VLDs) in proximity with the fat tissue sample, while the photo-activation index (PAI) thereof is photometrically-measured between periodically-alternating modes of photo-activation, so as to ensure that the fat tissue is optimally photo-activated, and over-activation is avoided.

Another object is to provide a new and improved method of and system and network for integrating stem cell storage banks and cellular differentiation and enrichment programs.

Another object is to provide a new and improved method of and apparatus for treating collected fat tissue samples, including stem cells therein, using dermal injections to treat of one or more conditions selected from the group consisting of: treating anti-aging, lines and/or wrinkles; achieving re-volumization of tissue; treating acne and scar repair; and treating burns and chronic ulcers.

Another object is to provide a new and improved method of and apparatus for treating collected fat tissue samples, including stem cells therein, so as to derive differentiated stem cell lines for use in treating of one or more conditions selected from the group consisting of: treating a knee, an elbow, or arthritis; regeneration of cardiac muscle tissue; repair of neurologic injury such as spinal cord injury, or stroke; regeneration of cartilage for knees and hips; regeneration of cervical or lumbar disk regeneration.

These and other objects will become apparent hereinafter and in the claims.

### BRIEF DESCRIPTION OF THE DRAWINGS

In order to more fully understand the Objects, the following Detailed Description of the Illustrative Embodiments should be read in conjunction with the accompanying figure Drawings in which:

FIG. 1 is a schematic representation illustrating the known fact that adipocyte derived stem cell (ASC) are pluripotential, i.e. having the capacity for self-replication, (adipocytes, chondrocytes, and osteocytes), and differentiation (cardiomyocytes, endothelial cells, myocytes, neuronal cells, and osteoblasts);

FIG. 2A is a schematic representation of the internetworked system for authenticating, photo-activating, assaying, cataloguing, tracking and managing aspirated fat tissue samples, including stem cells therein, in accordance with the principles of the present invention, for purposes of reinjection into patients so as to repair or construct skin, cartilage, bone, muscle and/or cardiac tissue;

FIG. 2B is a schematic representation of the process for authenticating, photo-activating, assaying, cataloguing, tracking and managing aspirated fat tissue samples including stem cells therein, in accordance with the principles of the present invention, for purposes of reinjection into patients so as to repair or construct skin, cartilage, bone, muscle and/or cardiac tissue;

FIG. 3A is a first perspective view of the RFID-tagged tissue collection device of the present invention having use in the different types of tissue aspiration instruments of the present invention disclosed herein, as well as in the different types of tissue processing instruments of the present invention, also disclosed herein;

FIG. 3B is an exploded view of the RFID-tagged tissue collection device of the present invention shown in FIG. 3A, and comprising (i) a cylindrical optically transparent tissue collection tube having micro-pores formed along one side of the optically transparent walls thereof, (ii) a micro-pore occluder that slides about the cylindrical tissue collection tube to selectively occlude the micro-pores when configured as shown in FIG. 4C and un-occlude the micro-pores when configured as shown in FIG. 4D, (iii) a distal cap for sealing off the open distal tip portion of the tissue collection tube, (iv) proximal cap for sealing off the open proximal end opening of the tissue collection tube; and (v) an RFID tag applied to the cylindrical tissue collection tube;

FIG. 4A is a perspective view showing the RFID-tagged tissue collection and processing device of the illustrative embodiment, comprising (i) a cylindrical optically transparent tissue collection tube having micro-pores formed along one side of the optically transparent walls thereof, (ii) a micro-pore occluder that slides about the cylindrical tissue collection tube to selectively occlude the micro-pores when configured as shown in FIG. 4B, and un-occlude the micro-pores when configured as shown in FIG. 4C, (iii) a distal cap for sealing off the open distal tip portion of the tissue collection tube, and (iv) a plunger for insertion through the proximal opening of the tissue collection tube and into its interior cylindrical volume to express a collected tissue sample out of its distal end opening;

FIG. 4B is a perspective view of the tissue collection and processing device of FIG. 4A shown configured in its micro-pores in its occluded state;

FIG. 4C is a perspective view of the tissue collection and processing device of FIG. 4A shown configured in its micro-pores in its occluded state, and its distal end portion sealed with a distal cap;

FIG. 5 is a perspective view of the tissue collection and processing device of FIG. 4C having a cannula connected to its distal end opening via Leur-lock connector;

FIG. 6A is a perspective view of a hand-supported power-assisted tissue aspiration instrument connected to an in-line tissue collection device containing six RFID-tagged tissue collection tubes of the present invention;

FIG. 6B is an exploded view of the in-line tissue collection device shown in FIG. 6A;

FIG. 7 is a schematic illustration indicating the beneficial effects of low level photo-active light energy on cellular tissue, including proliferation, migration and adhesion, thus increasing the rate of tissue transplantation and grafting;

FIG. 8A is a schematic illustration indicating that there exists an optical window into cells over the red and near-red light band (i.e. between 600-900 nanometer wavelengths), where light photo-active energy over this photo-active band is received by photo-acceptors (i.e. chromophores) in the mitochondrial regions of the cell, to influence the respiratory chain including the production of Cytochrome C Oxidase (Cco) and increase the bio-energy state of the cell;

FIG. 8B is a schematic representation of the absorbance versus wavelength response characteristics of chromophores within cellular tissue;

FIG. 8C is a schematic representation of the Reactive Oxygen Species (ROS) and gene transcription within cellular tissue;

FIG. 8D is a graphical representation illustrating the nitric oxide (NO) versus wavelength absorbance characteristics over infrared portion of the electromagnetic energy spectrum;

FIG. 8E is a schematic representation of the Arndt-Schulz biphasic response curve, indicating that there is an optimum photo-activation dosage that can be delivered and monitored;

FIG. 9A is a schematic representation of the photometrically-controlled photo-activation instrument system, for treating aspirated fat tissue samples, in accordance with the principles of the present invention, shown comprising (i) a photometrically-controlled photo-activation chamber adapted to receive at least one RFID-tagged sealed tissue collection device for photo-activation treatment using a photometrically-controlled photo-activation process supported by the instrument, (ii) RFID tag read/write subsystem for reading from and writing to the RFID tag on the tissue collection device, (iii) a photo-activation illumination subsystem for illuminating the aspirated tissue sample including stem cells contained in the tissue collection and processing device, (iv) a real-time photo-activation index (PAI) measurement subsystem for measuring the PAI of the collected tissue sample during and after photo-activation operations, determining the rate of change of this index and ceasing photo-activation when this rate of change is essentially zero or is closely approaching this limit, (v) an information display subsystem for displaying information to the doctor and other medical personnel during instrument operation; (vi) a memory subsystem for storing and retrieving information regarding tissue samples collected and processed by the instrument system, or associated with a tissue banking system, and (vii) an input/output (I/O) subsystem for interfacing the instrument system with one or more host systems and/or wired and/or wireless data communication networks;

FIG. 9B is a graphical illustration of a prophetic example of the photo-activation response characteristics of an in vitro aspirated tissue sample, showing the photo-activation index (AI) increases with low level photo-active light energy exposure, and then decreases after a particular amount of LLW energy exposure;

FIGS. 9C1 and 9C2, taken together, set forth a flow chart describing the primary steps of a first illustrative in vitro method of photo-activating a collected sample of aspirated fat tissue including stem cells contained therein using photometric (i.e. nitric-oximetry) feedback principles supported by the instrument system shown in FIG. 9A;

FIGS. 9D1 and 9D2, taken together, set forth a flow chart describing the primary steps of a second illustrative in vitro method of photo-activating a collected sample of aspirated fat tissue including stem cells contained therein using photometric (i.e. nitric-oximetry) feedback principles supported by the instrument system shown in FIG. 9A;

FIG. 10A is a schematic representation showing a first illustrative embodiment of the tissue authentication and photo-activation instrument system of present invention, capable of authenticating and photo-activating a collected fat tissue sample including stem cells contained in a sealed tissue collection and processing device inserted within its photometrically-controlled photo-activation chamber, mounted within the top surface of a console unit having instrument controls, an LCD touch-screen display panel, data entry keypad and the like;

FIG. 10B is a block schematic representation of the tissue authentication and photo-activation instrument system shown in FIG. 10A;

FIG. 10C is a perspective view of the sealed tissue collection device shown in FIG. 3A, and for insertion with the photometrically-controlled photo-activation chamber installed within the tissue authentication and photo-activation instrument system of FIG. 10A;

FIG. 10D is a perspective view the photometrically-controlled photo-activation chamber of FIG. 10A, showing the sealed tissue collection tube of FIG. 10C inserted within the central treatment volume of the lensed barrel insert installed against the interior walls of the photometrically-controlled photo-activation chamber;

FIG. 10E1 is a perspective view of the lensed barrel insert installed within the photometrically-controlled photo-activation chamber of FIG. 10D;

FIG. 10E2 is a cross-sectional view of the lensed barrel insert shown in FIG. 10E1, taken along line 10E2-10E2;

FIG. 10F is a schematic representation of the process used to compute the photo-activation index (PAI) of an aspirated tissue sample at any testing interval within the instrument system of FIG. 10A;

FIG. 10G1 is a cross-sectional view of the photometrically-controlled photo-activation chamber of FIG. 10D, taken along the line 10G1-10G1 thereof;

FIG. 10G2 is a cross-sectional view of the photometrically-controlled photo-activation chamber of FIG. 10D, taken along the line 10G2-10G2 thereof;

FIG. 10H is a timing diagram illustrating the periodic photo-activation treatment periods during the photo-activation modes/states of the instrument system of FIG. 10A, and the periodic photometric measurement periods during the photometric modes of the instrument system;

FIG. 11A is a schematic representation showing a second illustrative embodiment of the tissue authentication and photo-activation instrument system of present invention, capable of authenticating and photo-activating a aspirated fat tissue sample contained in a sealed tissue collection tube that is inserted within the photometrically-controlled photo-activation chamber mounted within a hand-held device that is electrically connected to a console unit having controls, a display panel, and data entry keypad;

FIG. 11B is a block schematic representation of the tissue authentication and photo-activation instrument system shown in FIG. 11A;

FIG. 11C is a first perspective view of the hand-held photo-activation device of FIG. 11A, showing treating a sample of aspirated tissue contained within a sealed tissue collection device inserted within the photometrically-controlled photo-activation chamber of the hand-held photo-activation device;

FIG. 11D is a schematic representation of the process used to compute the photo-activation index of an aspirated tissue sample at any testing interval within the instrument system of FIG. 11A

FIG. 11E1 is a cross-sectional view of the photometrically-controlled photo-activation chamber of FIG. 11C, taken along the line 11E1-11E1 thereof;

FIG. 11E2 is a cross-sectional view of the photometrically-controlled photo-activation chamber of FIG. 11C, taken along the line 11E2-11E2 thereof;

FIG. 11F is a timing diagram illustrating the periodic photo-activation treatment periods during the photo-activation modes/states of the instrument system of FIG. 11A, and the periodic photometric measurement periods during the photometric modes of the instrument system;

FIG. 12A is a schematic representation showing a third illustrative embodiment of the tissue photo-activation instrument system of present invention, capable of authenticating and photo-activating a collected fat tissue sample contained in a sealed tissue collection device inserted within a hand-held tissue injector gun, and enveloped within its photometrically-controlled photo-activation chamber, and operably connected mounted to a console unit having controls, a display panel, and data entry keypad;

FIG. 12B is a block schematic representation of the tissue authentication and photo-activation instrument system shown in FIG. 12A;

FIG. 12C is a perspective view of the hand-held tissue injector gun employed in the tissue authentication and photo-activation instrument system of FIG. 12A, with its photo-activation/photometric pod removed from about the loaded tissue collection device;

FIG. 12D is an elevated semi-transparent perspective view of the hand-held tissue injector gun employed in the tissue authentication and photo-activation instrument system of FIG. 12A;

FIG. 12E is a schematic representation of the process used to compute the photo-activation index of an aspirated tissue sample at any testing interval within the instrument system of FIG. 12A

FIG. 12F1 is a cross-sectional view of the photometrically-controlled photo-activation chamber of shown in FIG. 12D, taken along the line 12F1-12F1 thereof;

FIG. 12F2 is a cross-sectional view of the photometrically-controlled photo-activation chamber of shown in FIG. 12D, taken along the line 12F2-12F2 thereof;

FIG. 12G is a timing diagram illustrating the periodic photo-activation treatment periods during the photo-activation modes/states of the instrument system of FIG. 12A, and the periodic photometric measurement periods during the photometric modes of the instrument system;

FIG. 13A is a schematic representation showing a fourth illustrative embodiment of the tissue photo-activation instrument system of present invention, capable of photo-activating fat tissue in vivo using a hand-held photo-activation instrument having integrated controls, and touch-screen display panel;

FIG. 13B is a block schematic representation of the tissue photo-activation instrument system shown in FIG. 13A;

FIG. 13C is a schematic representation of the process used to compute the photo-activation index of an aspirated tissue sample at any testing interval within the instrument of FIG. 13A;

FIG. 13D is a cross-sectional view of the photo-activation/photometric assembly employed in the instrument of FIG. 13A, taken along the line 13D-13D thereof;

FIG. 13E is a timing diagram illustrating the periodic photo-activation treatment periods during the photo-activation modes/states of the instrument system of FIG. 13A, and the periodic photometric (Photo-Activation Index) measurement periods during the photometric modes of the instrument;

FIG. 14A is a schematic representation showing a fifth illustrative embodiment of the tissue authentication and photo-activation instrument system of present invention, realized in a wireless mobile instrument form-factor, capable of authenticating and photo-activating an aspirated fat tissue sample including stem cells contained in a sealed tissue collection and processing tube inserted within a photometrically-controlled photo-activation chamber mounted within its hand-held housing, which is wirelessly connected to a battery charging and wireless data communication station, internetworked with the infrastructure of the Internet, including local area networks (LANs) and wide area network (WANs);

FIG. 14B is a block schematic representation of the tissue authentication and photo-activation instrument system shown in FIG. 14A;

FIG. 14C is a first perspective view of the hand-held photo-activation device of FIG. 14A, showing treating a sample of aspirated tissue including stem cells contained within a sealed tissue collection and processing device inserted within the photometrically-controlled photo-activation chamber of the hand-held photo-activation device;

FIG. 14D is a schematic representation of the process used to compute the photo-activation index (PAI) of an aspirated tissue sample at any testing interval within the instrument system of FIG. 14A;

FIG. 14E1 is a cross-sectional view of the photometrically-controlled photo-activation chamber shown in FIG. 14C, taken along the line 14E1-14E1 thereof;

FIG. 14E2 is a cross-sectional view of the photometrically-controlled photo-activation chamber shown in FIG. 14C, taken along the line 14E2-14E2 thereof;

FIG. 14F is a timing diagram illustrating the periodic photo-activation treatment periods during the photo-activation modes/states of the instrument system of FIG. 14A, and the periodic photometric measurement periods during the photometric modes of the instrument system;

FIG. 15 is a schematic representation describing the primary steps involved in carrying out the method of stem cell tissue harvesting, collecting, processing and injecting ASC-enriched tissue into patients for diverse modes of treatment in accordance with the principles of the present invention;

FIG. 16 is a schematic representation describing the primary steps involved in carrying out a first method of harvesting, concentrating and photo-activating tissue samples including stem cells therein, in accordance with the principles of the present invention; and

FIG. 17 is a schematic representation describing the primary steps involved in carrying out a second method of harvesting, concentrating and photo-activating tissue samples, including stem cells therein, in accordance with the principles of the present invention.

### DETAILED DESCRIPTION OF THE ILLUSTRATIVE EMBODIMENTS OF THE PRESENT INVENTION

Referring to the figures in the accompanying Drawings, the various illustrative embodiments of the apparatus and methodologies will be described in great detail, wherein like elements will be indicated using like reference numerals.

General Overview of The System and Network of The Present Invention

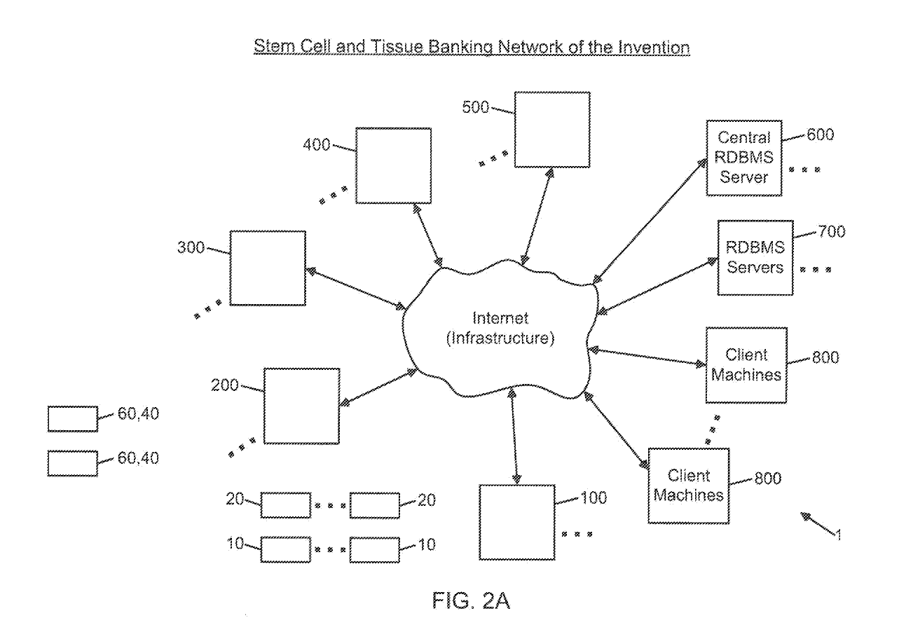

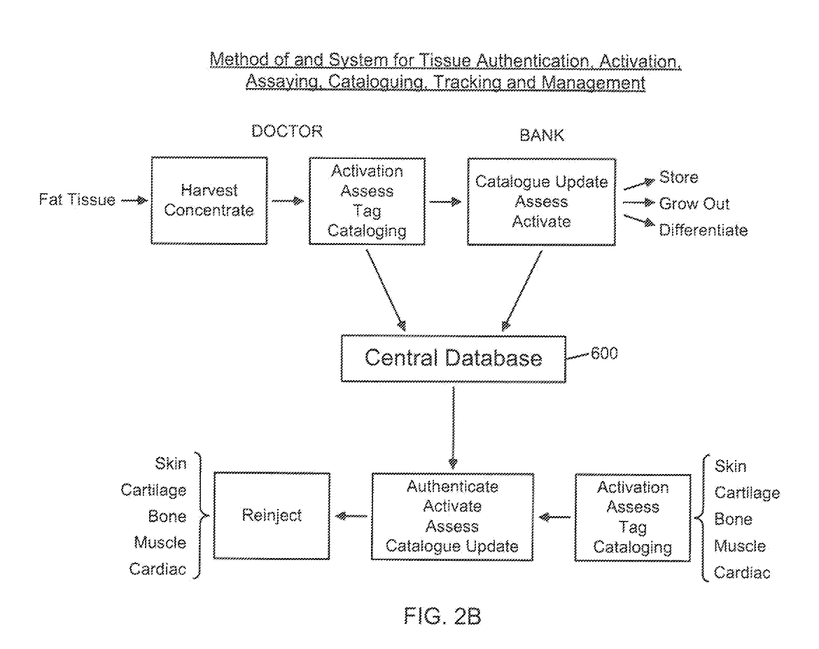

FIG. 2A provides an overview of the system and network of the present invention which supports authenticating, photo-activating, assaying, cataloguing, tracking and managing aspirated fat tissue samples in accordance with the principles of the present invention. Typically, such tissue samples are re-injected into patients to repair or construct skin, cartilage, bone, muscle and/or cardiac tissue.

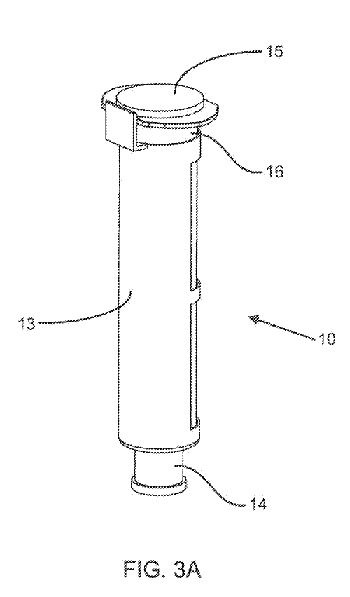

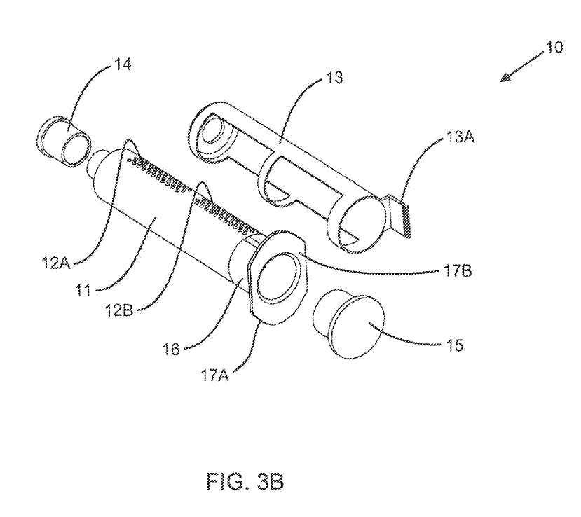

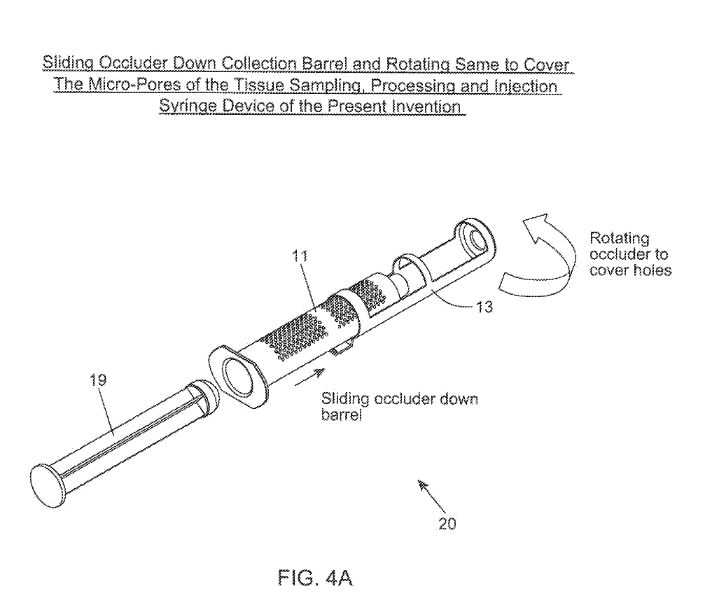

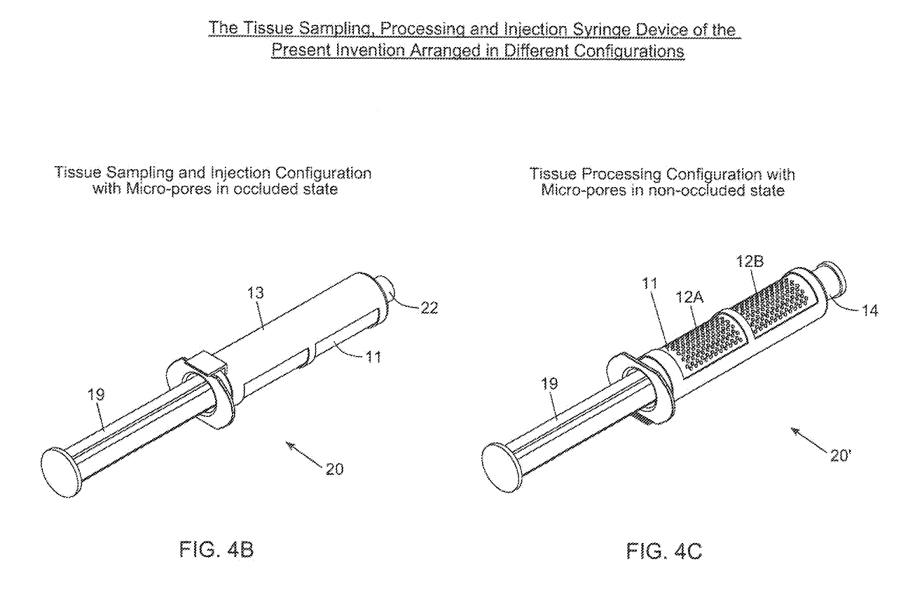

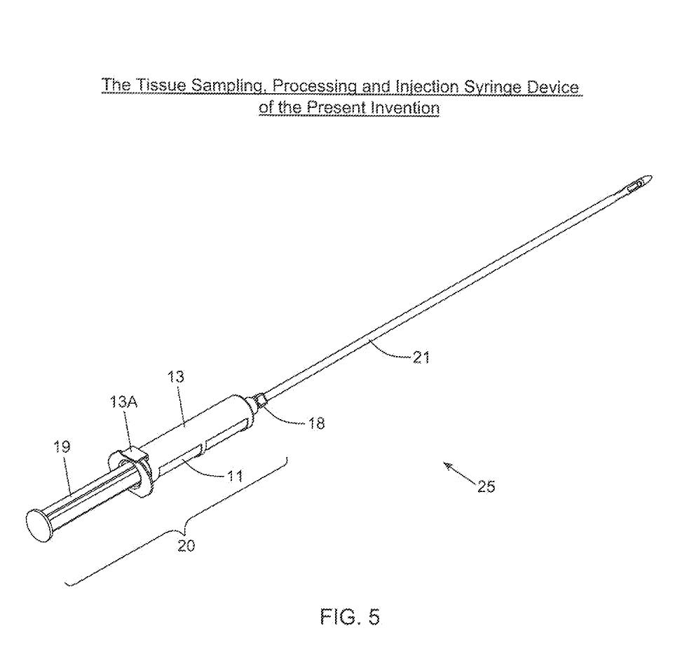

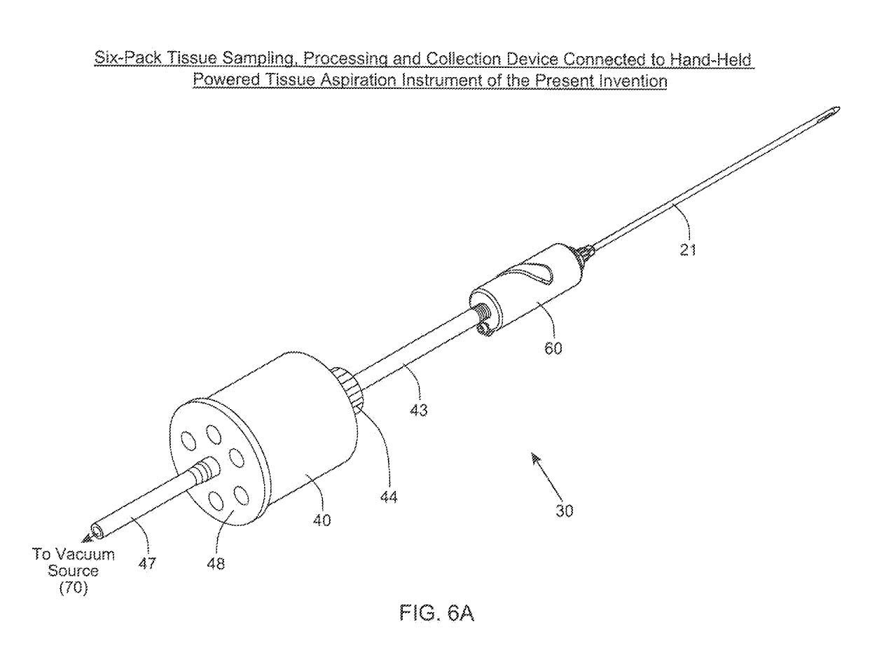

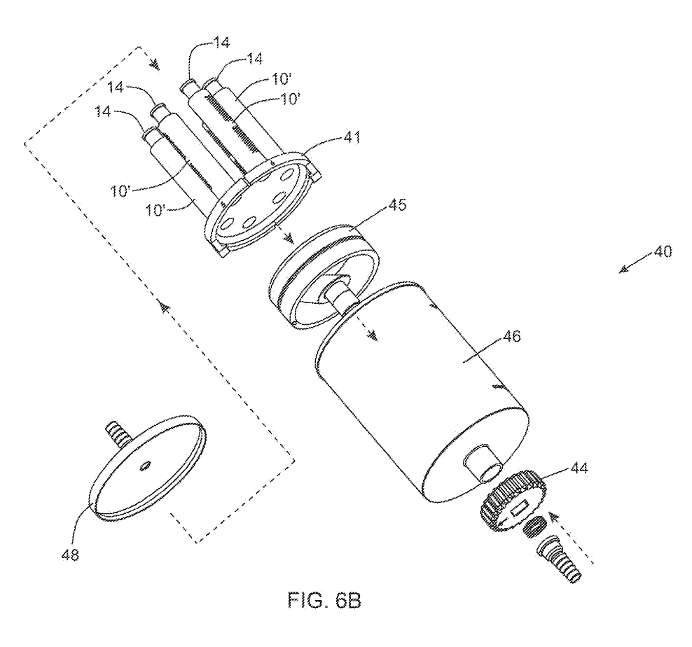

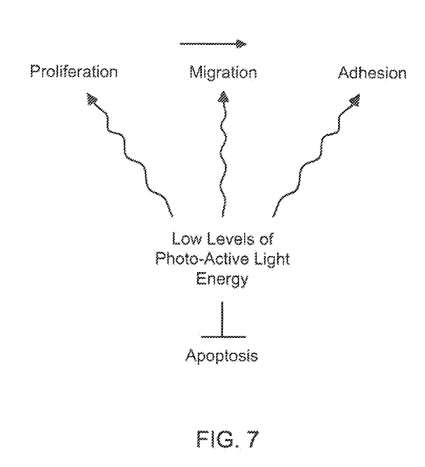

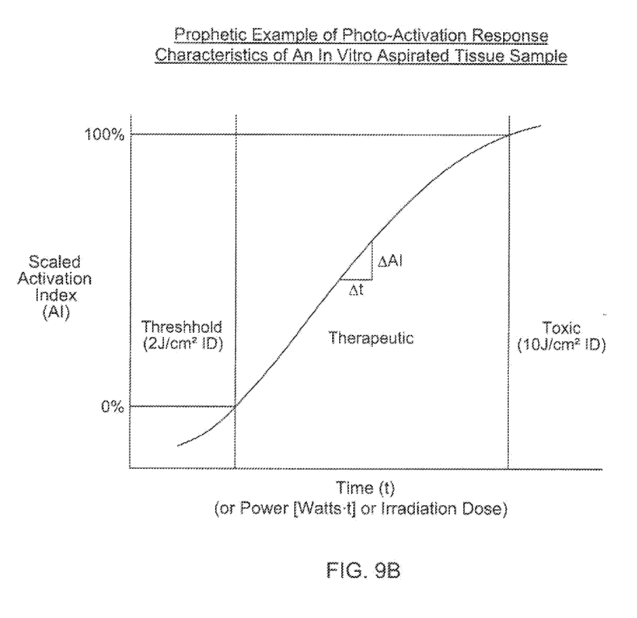

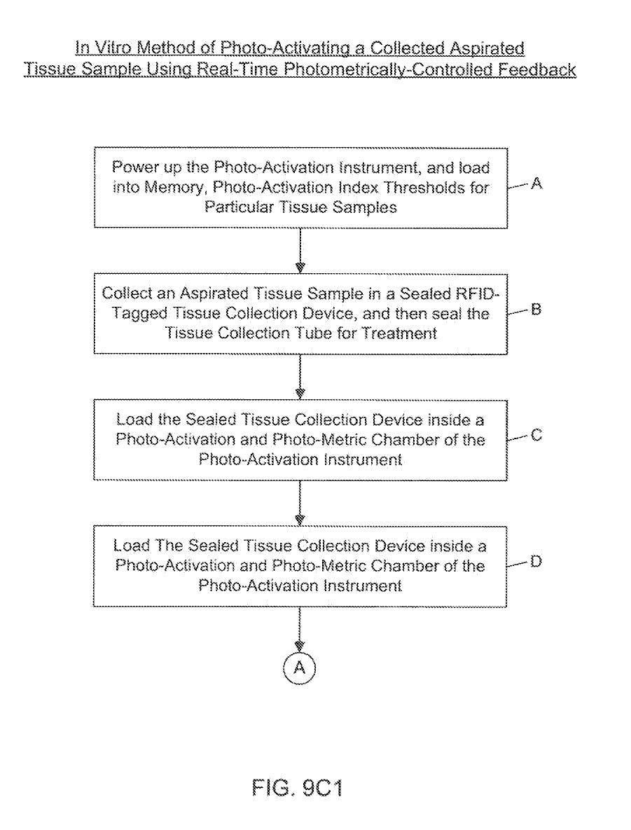

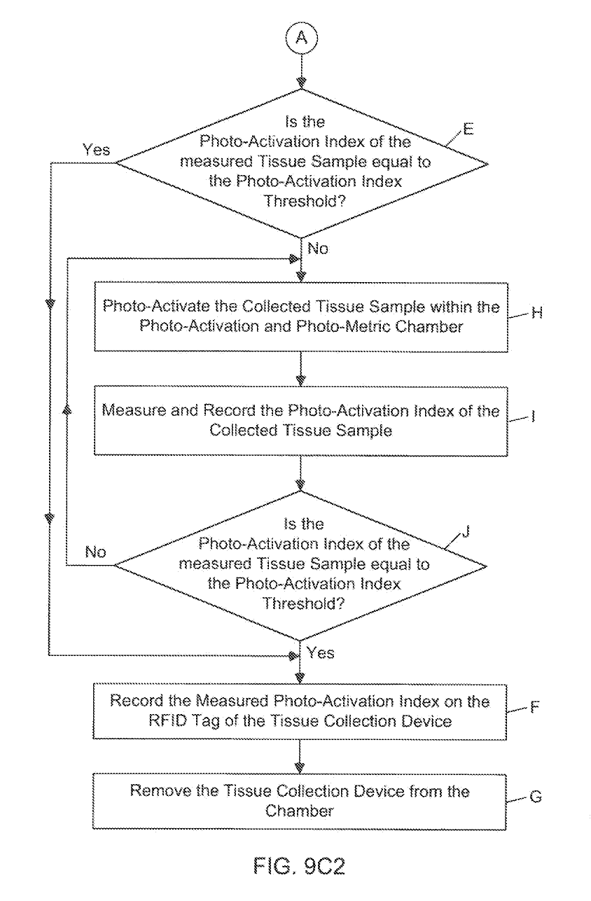

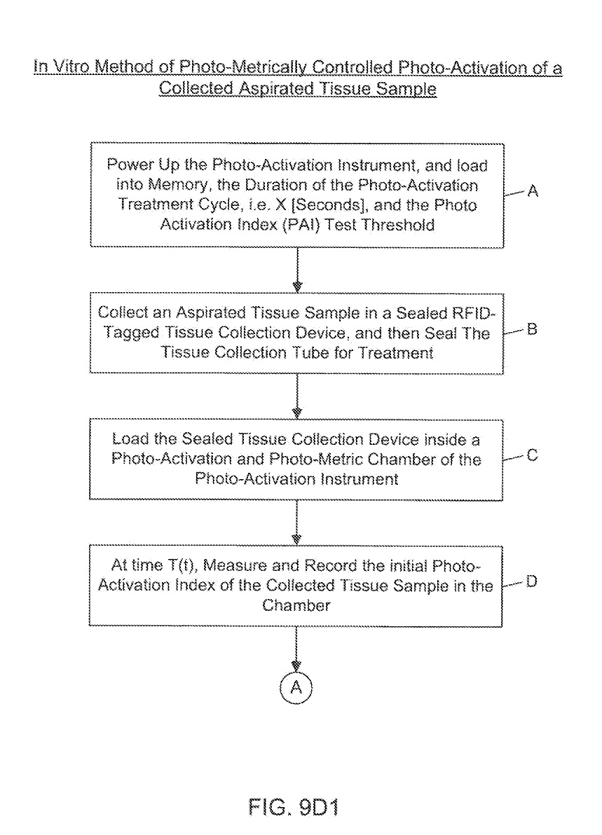

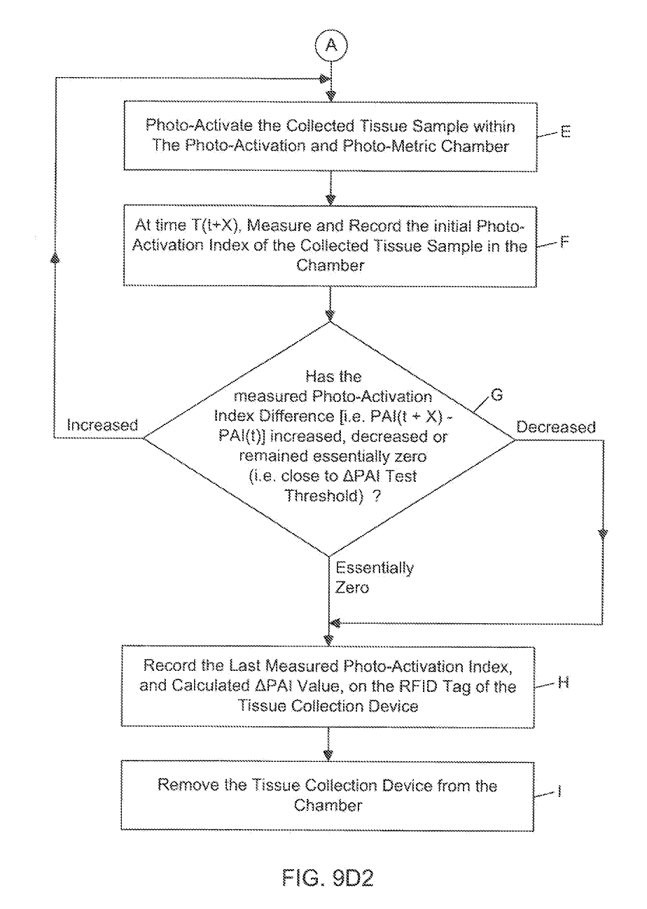

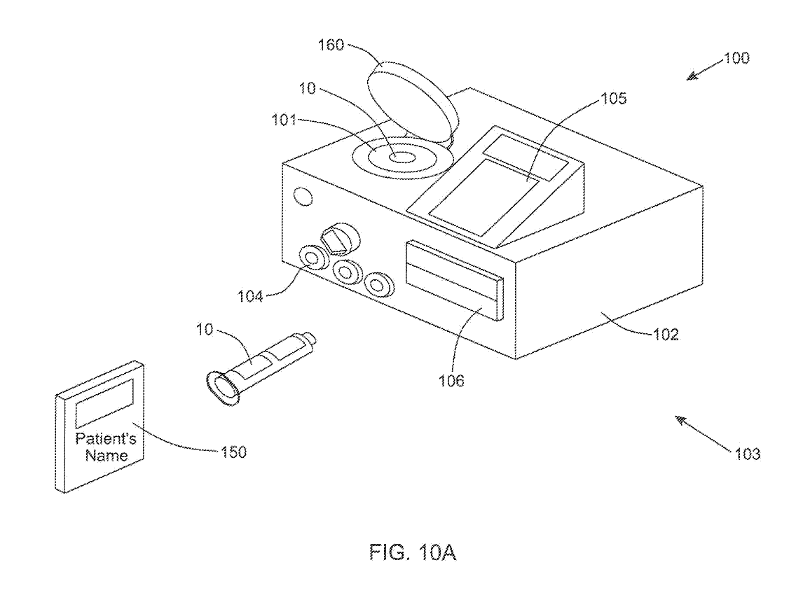

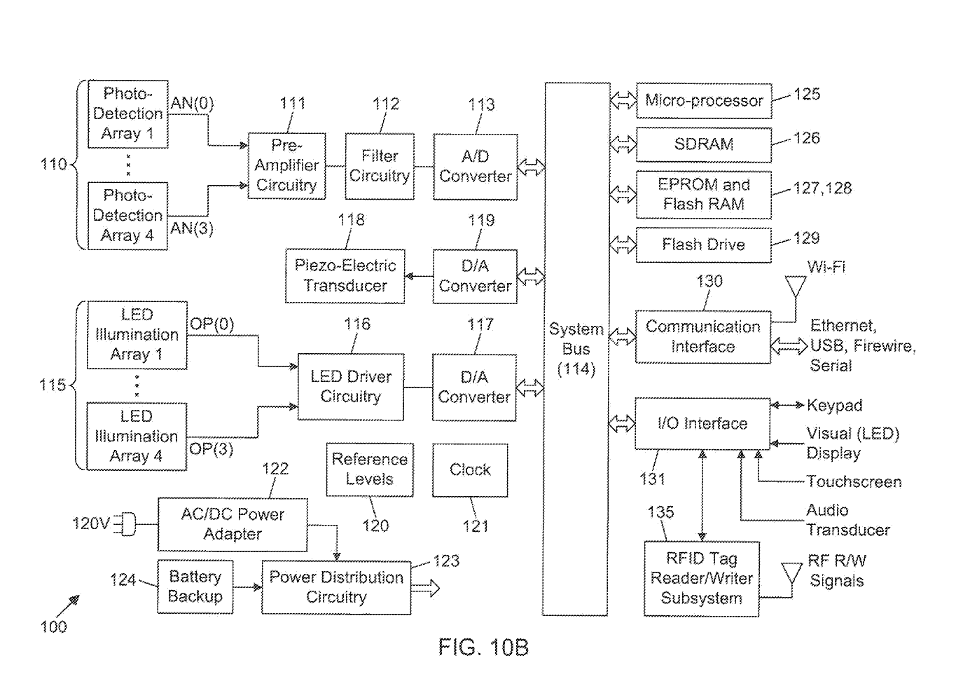

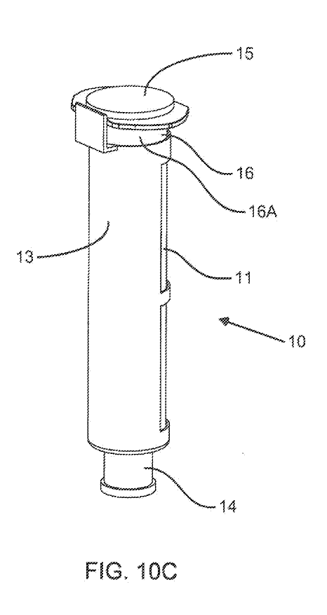

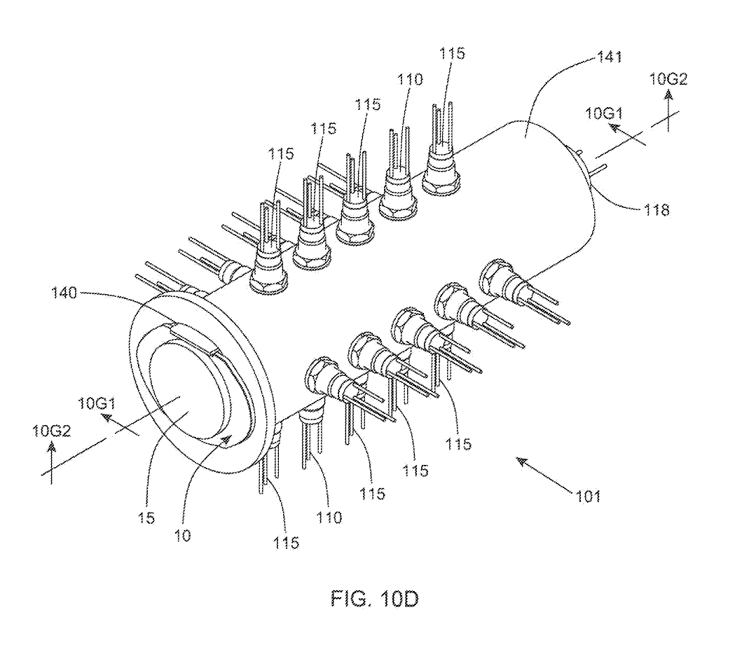

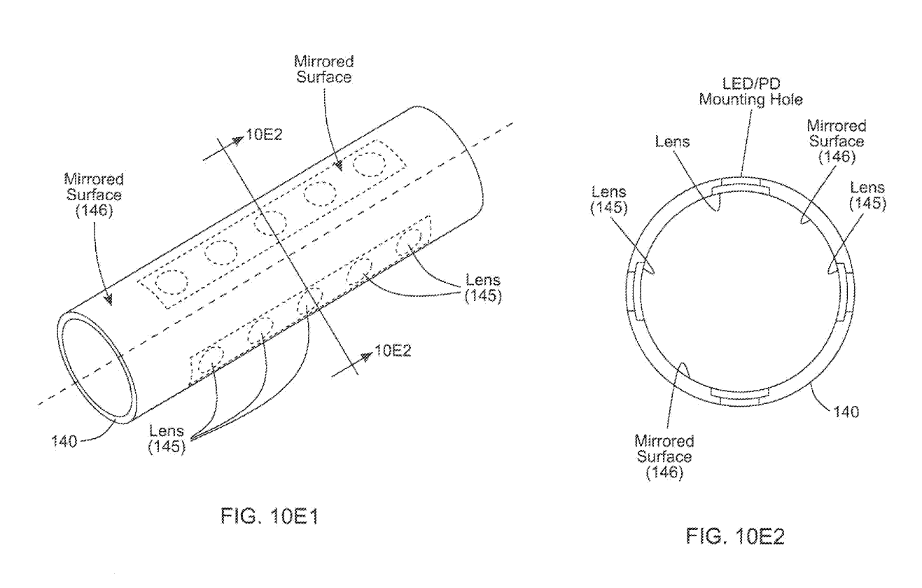

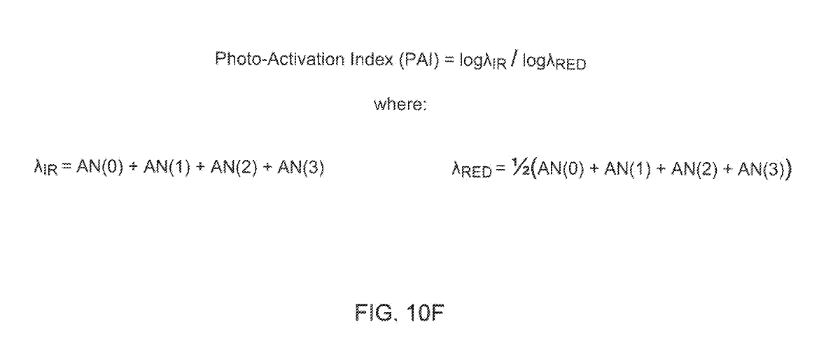

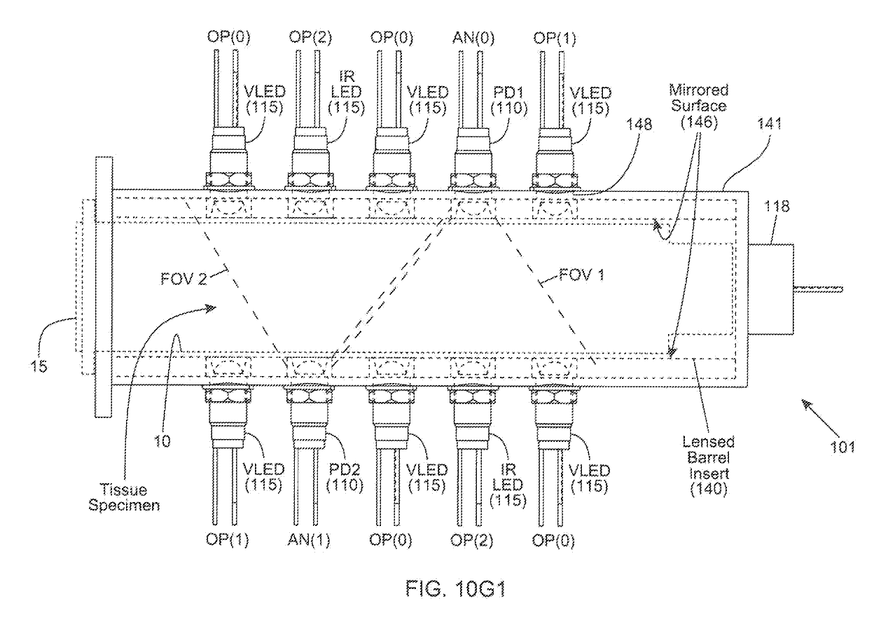

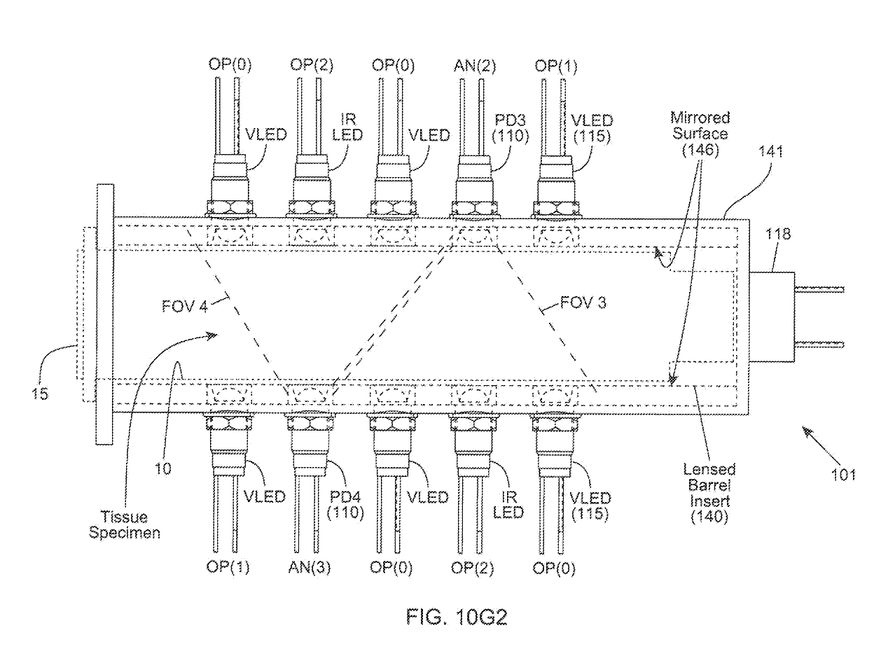

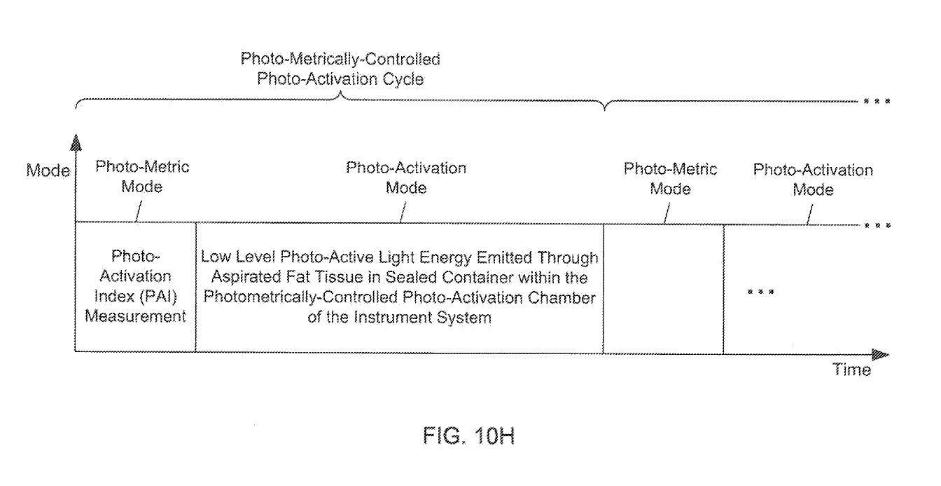

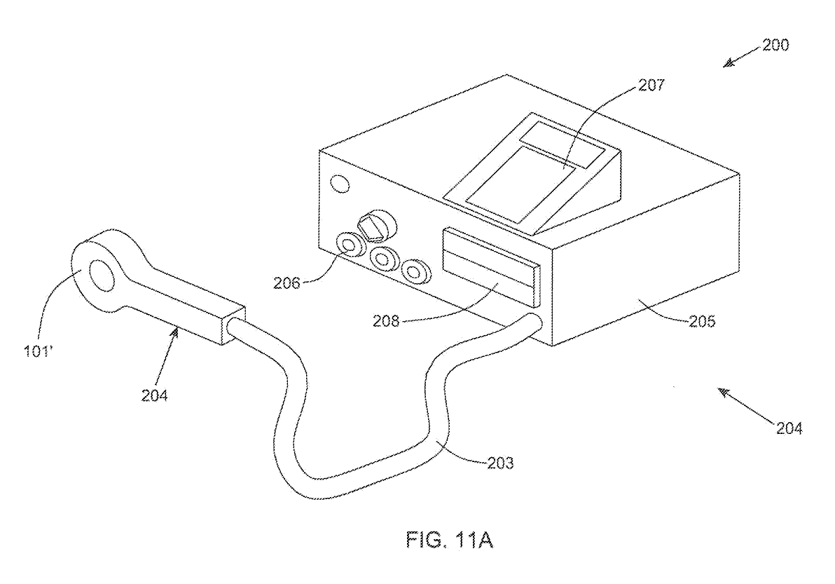

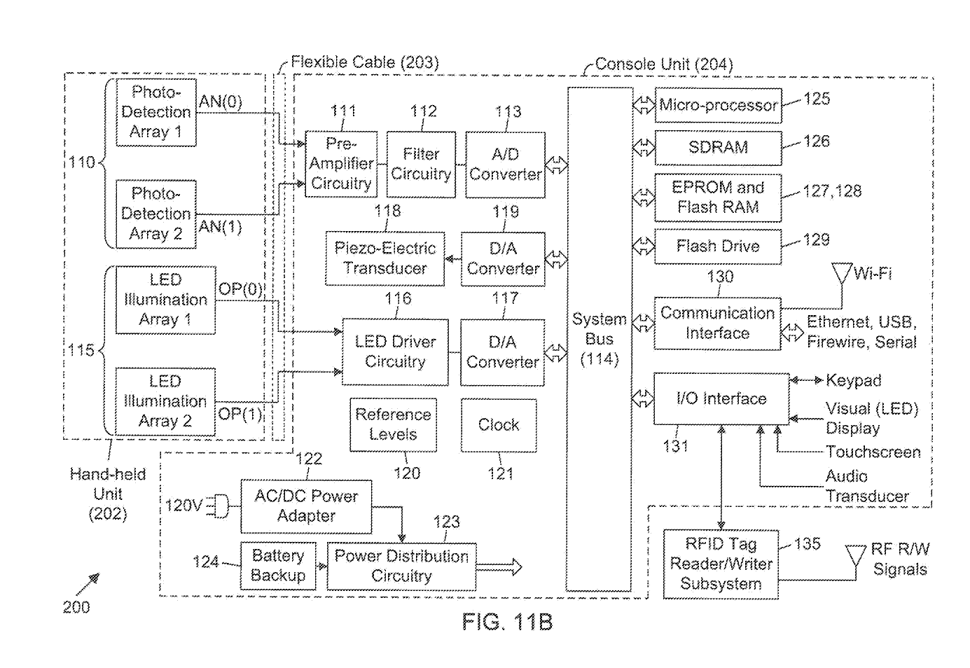

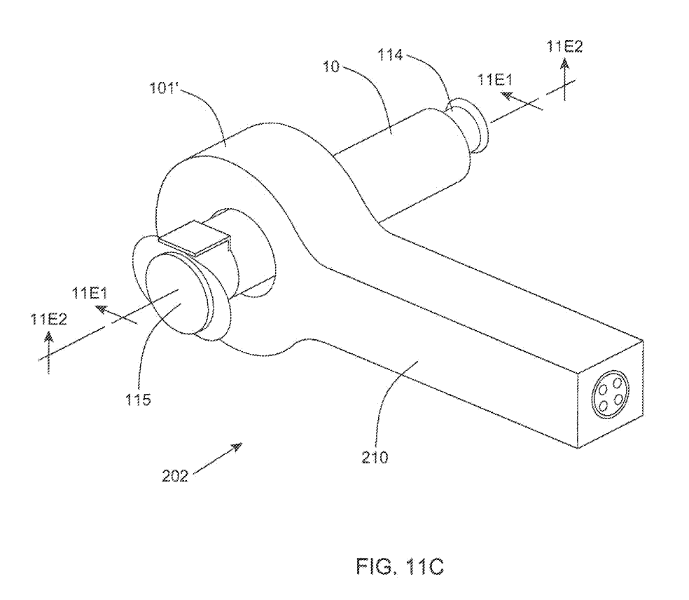

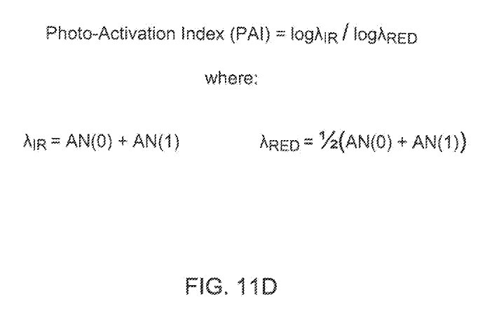

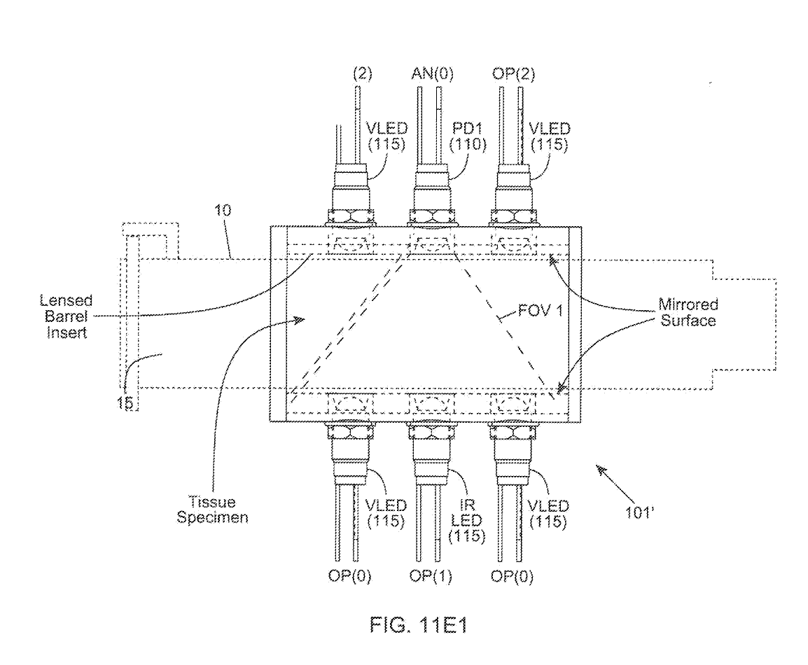

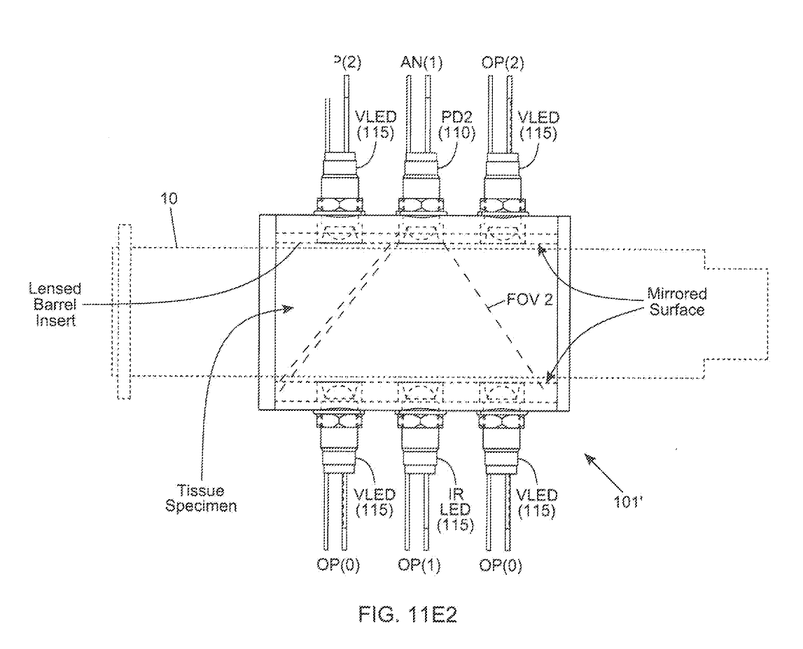

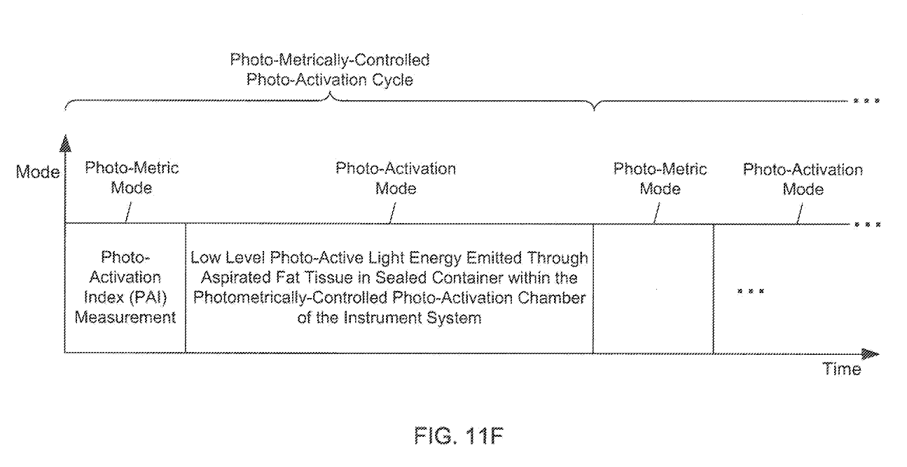

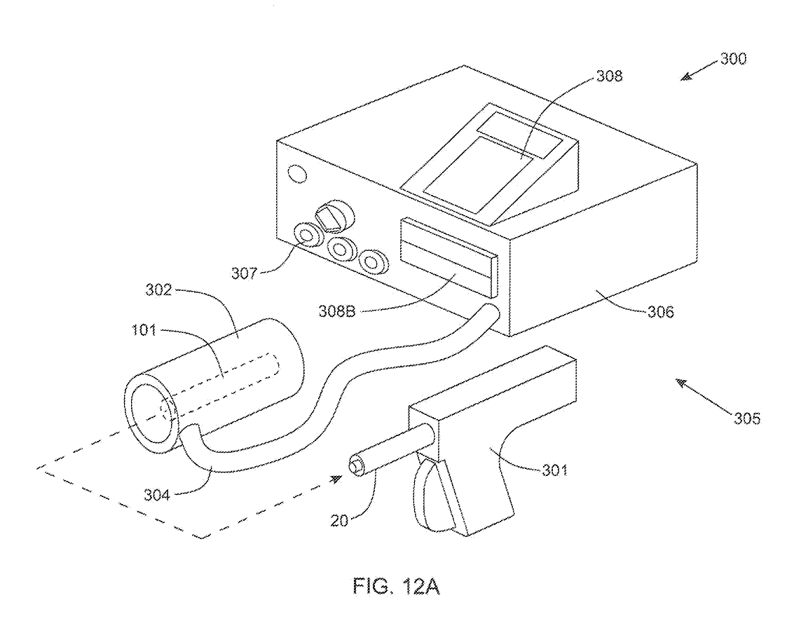

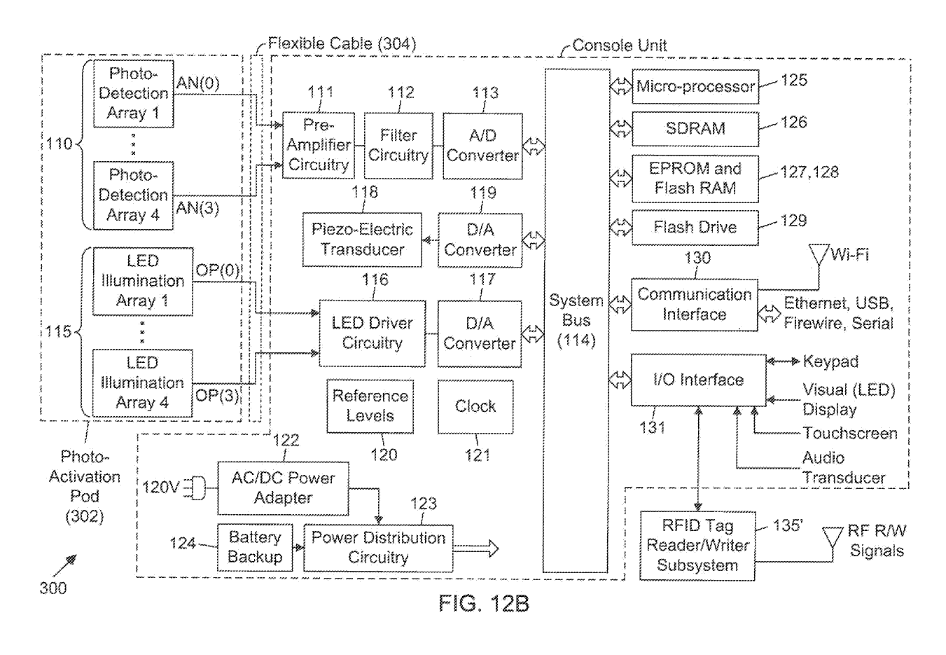

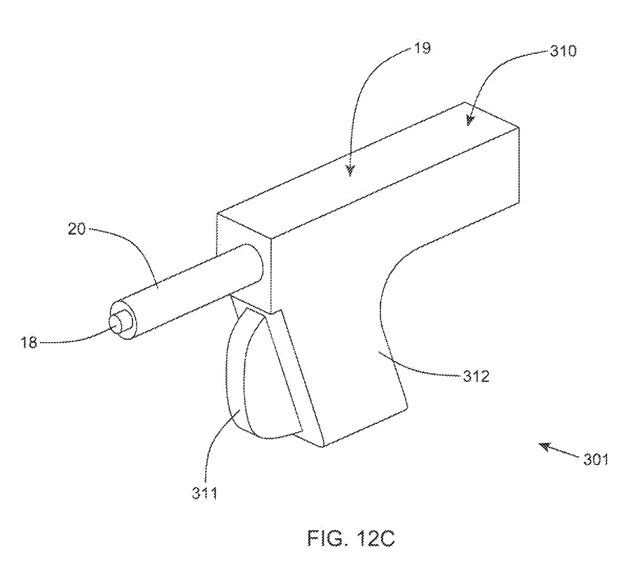

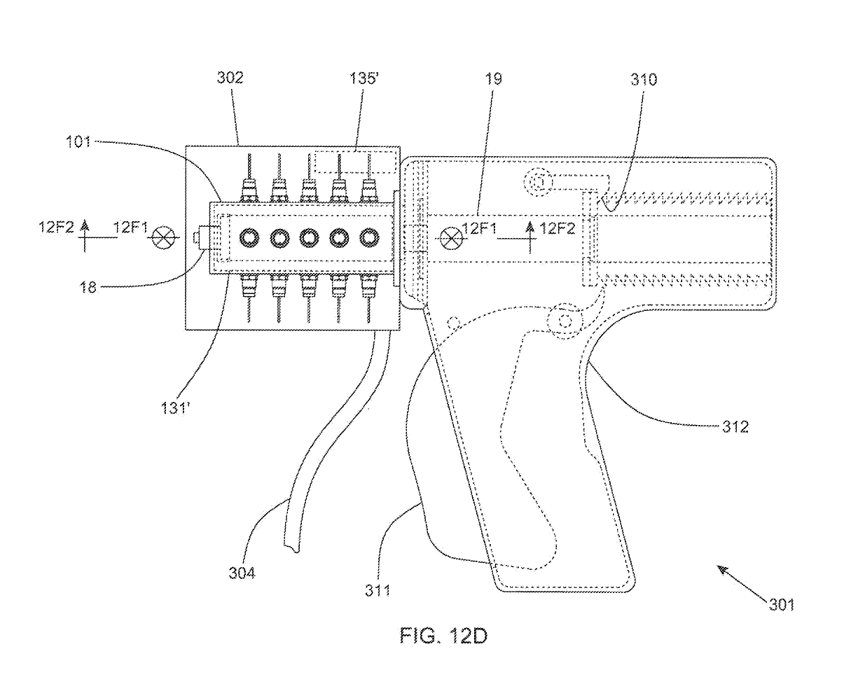

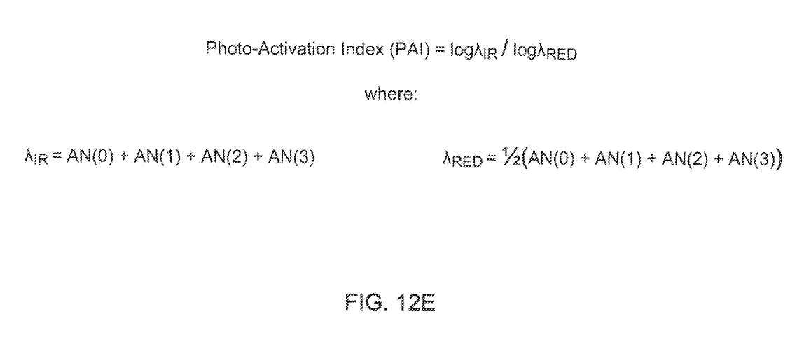

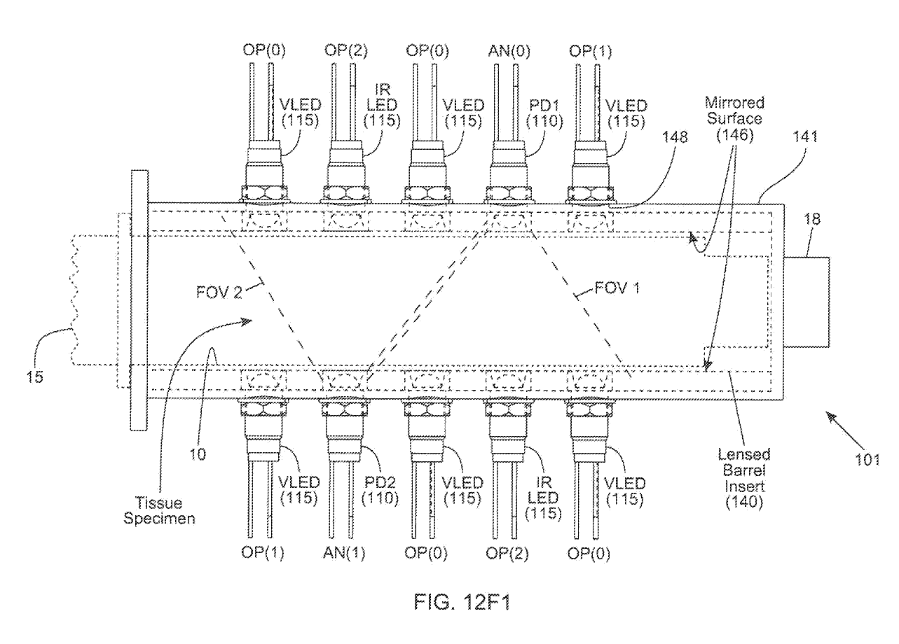

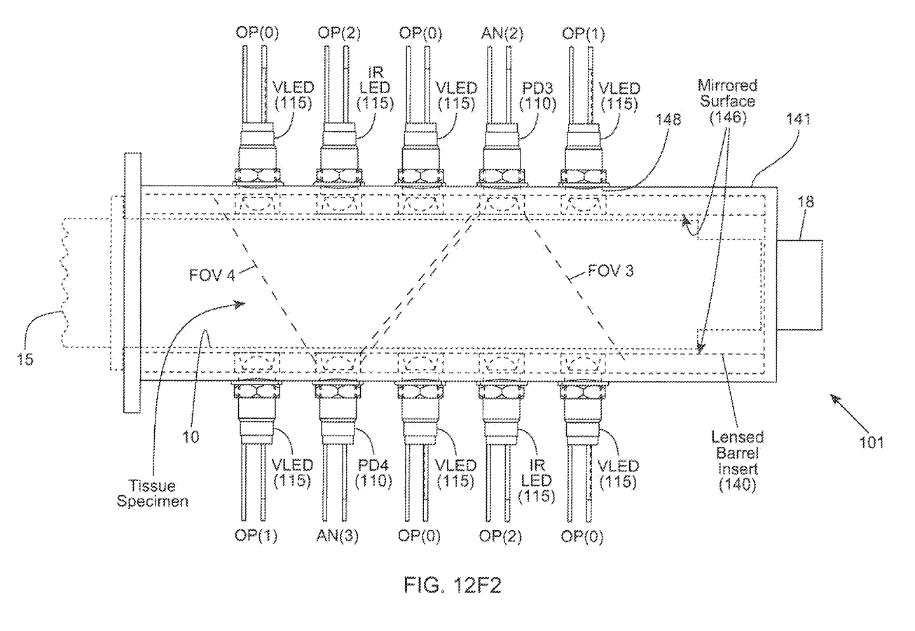

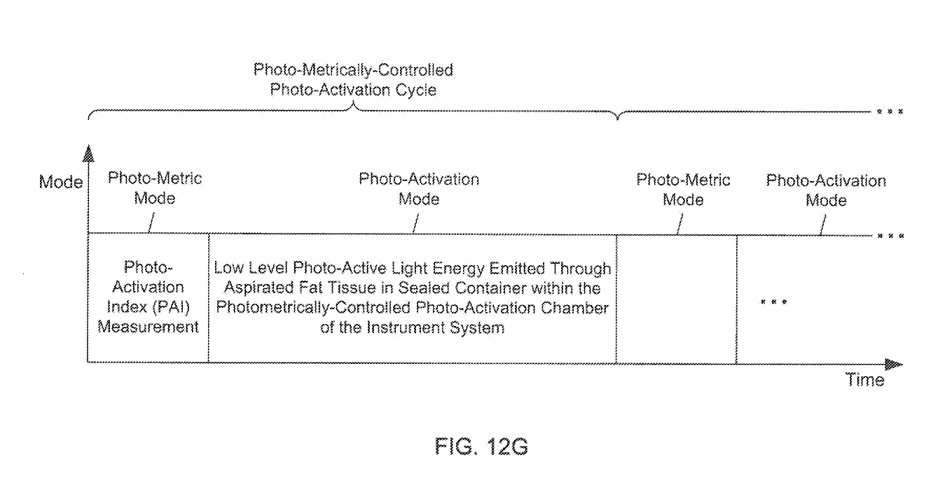

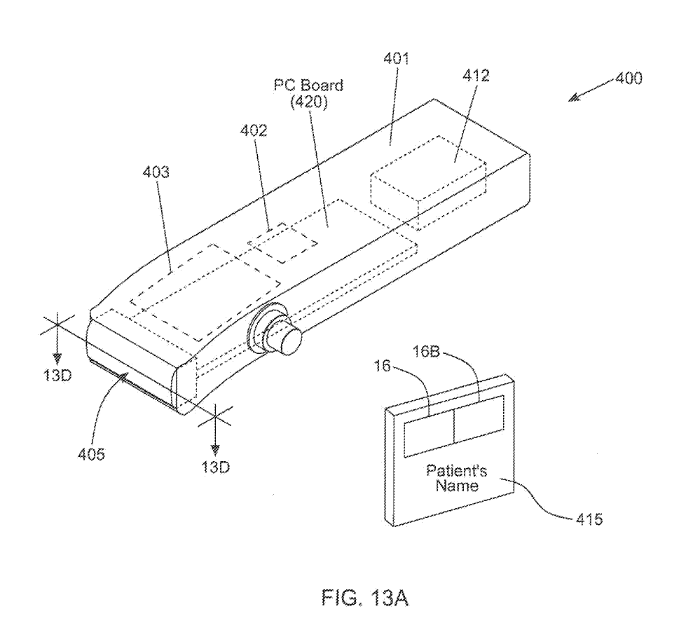

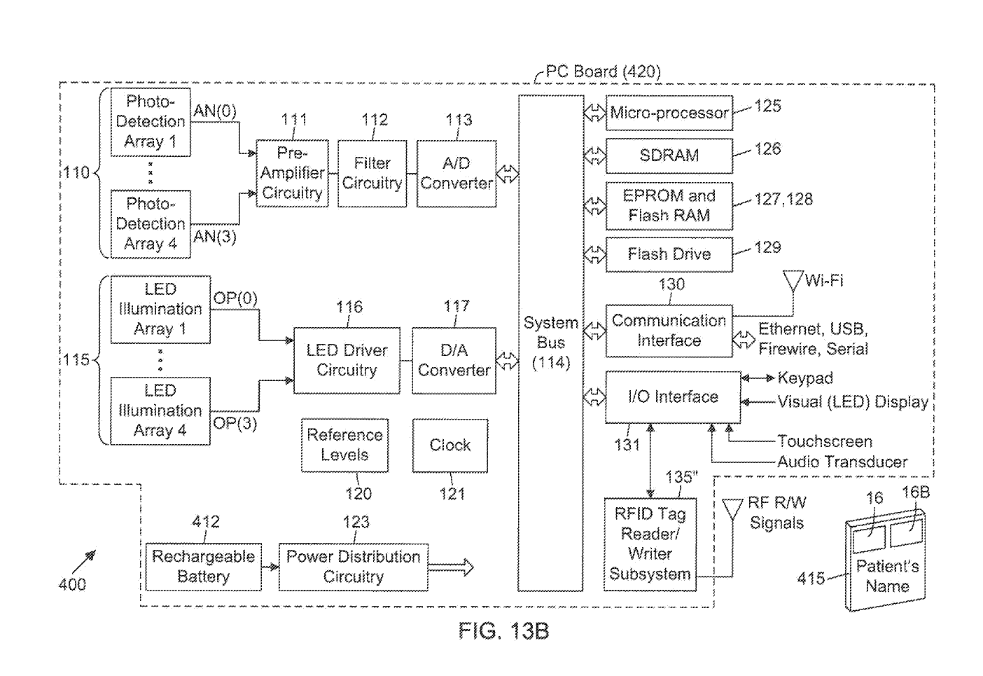

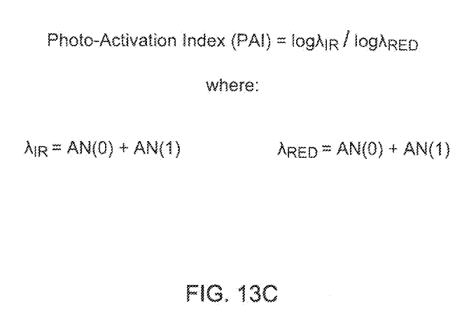

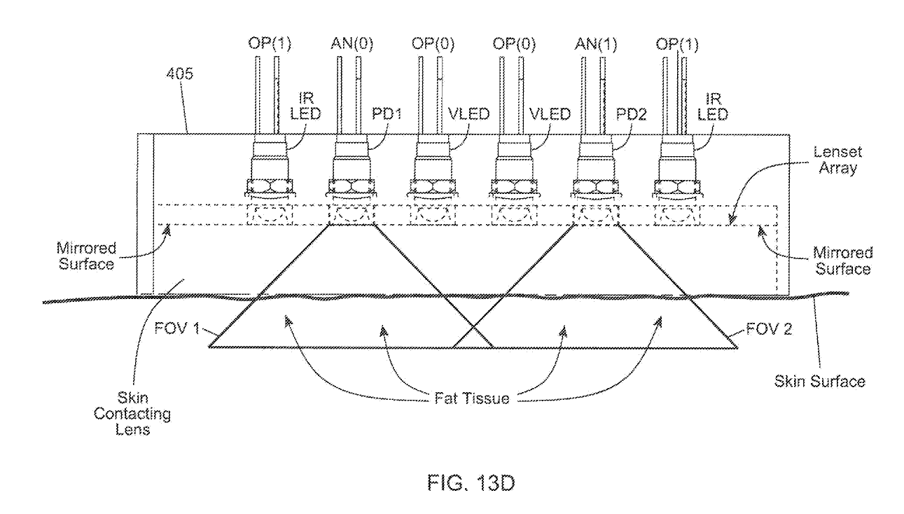

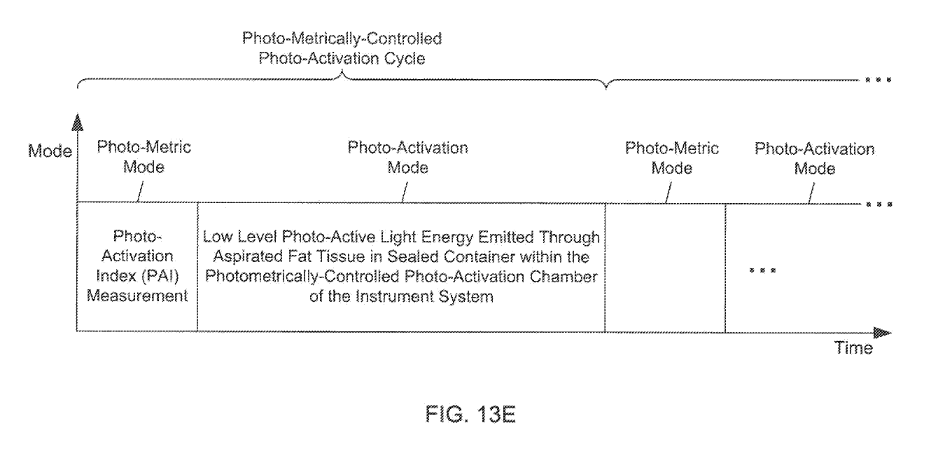

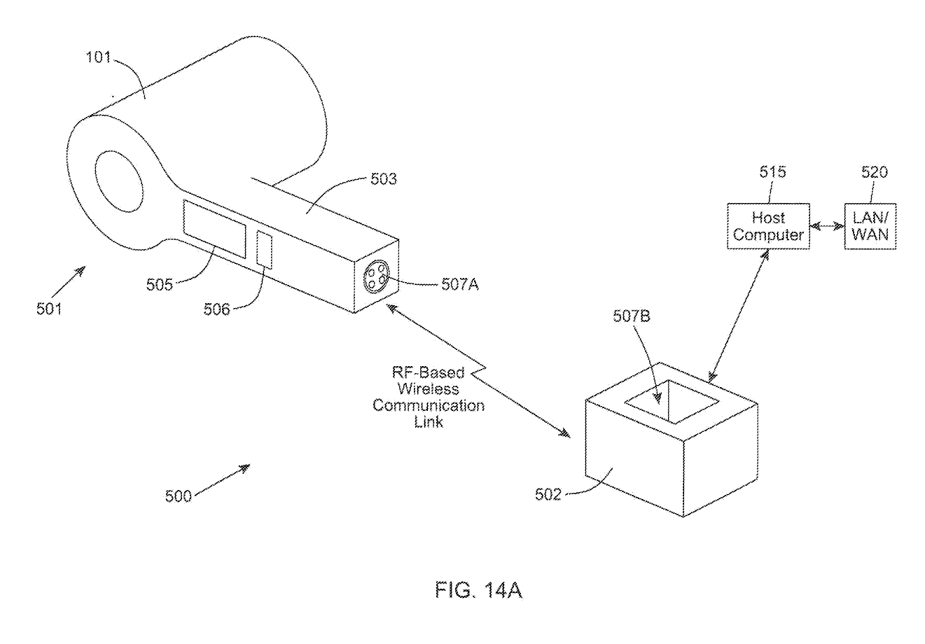

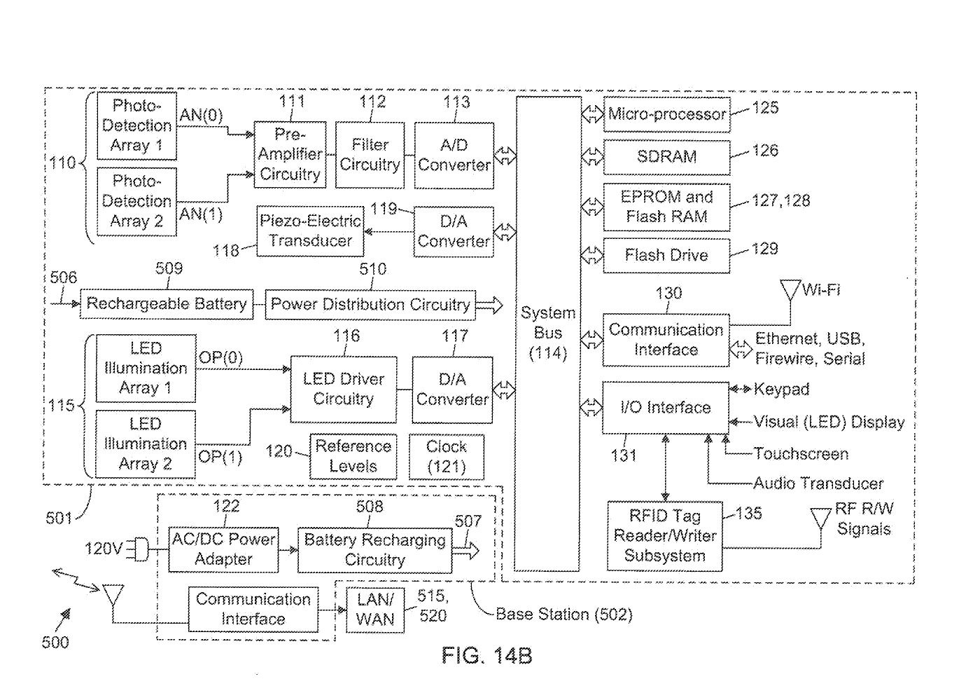

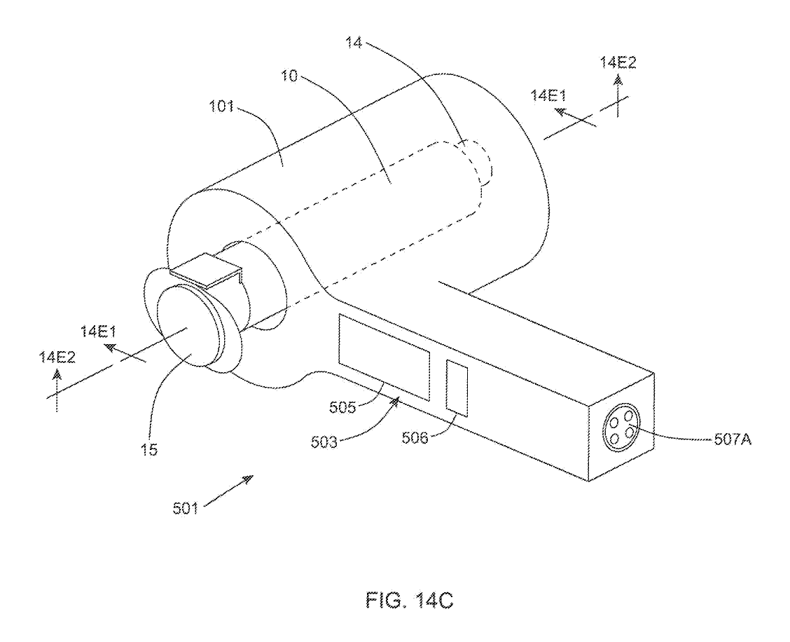

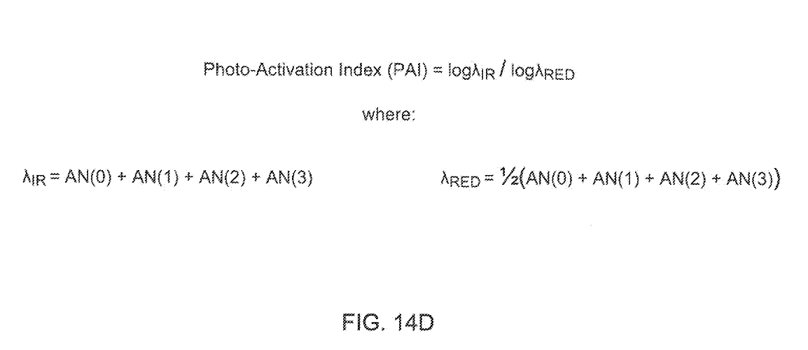

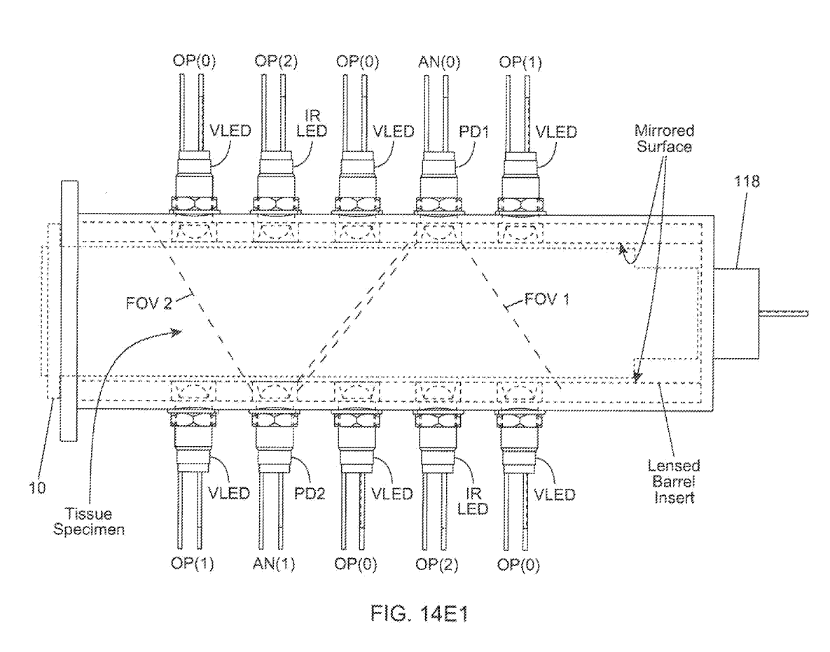

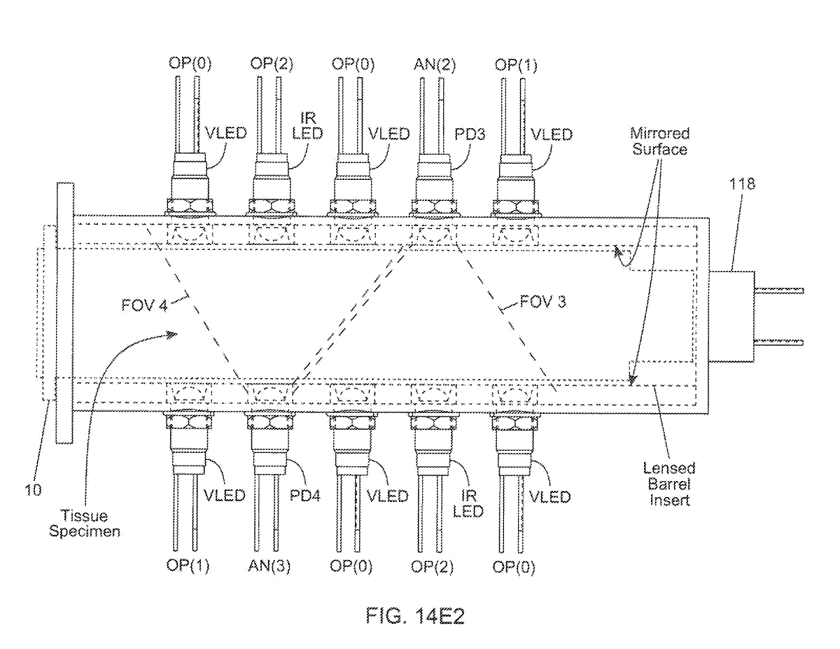

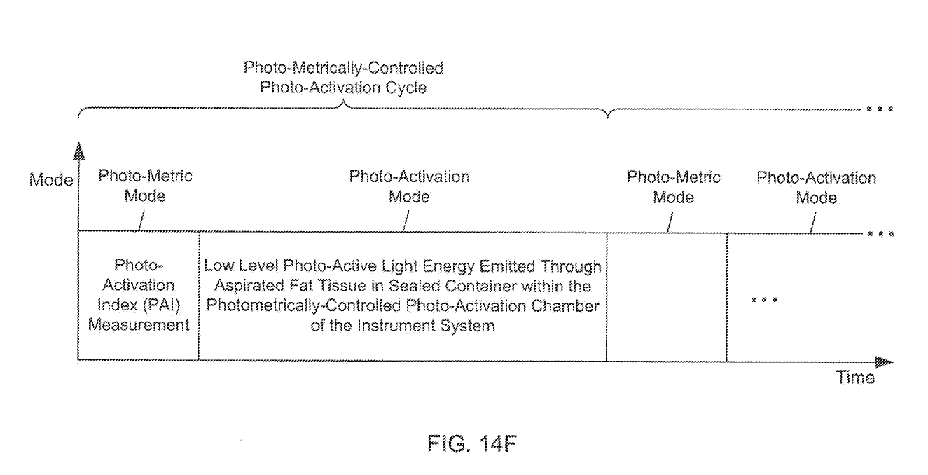

As shown in FIG. 2A, the internetworked system (i.e. network) of the present invention 1 generally comprises numerous system components typically located in physically different locations, namely: (i) a plurality of tissue collection and processing devices 10 as shown in FIGS. 3A through 3F, deployed in doctor's offices and operating rooms, and particularly adapted for use in (a) the manually-operated tissue aspiration and collection devices 20 shown in FIGS. 4A through 5, as well as (b) within in-line tissue collection devices 40 connected to power-assisted tissue aspiration instruments 60 as shown in FIGS. 6A and 6B, and throughout the instruments disclosed in Applicant's copending U.S. application Ser. No. 12/955,420 filed Nov. 29, 2010, supra; (ii) a plurality of countertop-based tissue authentication and photo-activation instrument systems 100, employing photometrically-controlled photo-activation chambers within the console housing, as described in FIGS. 10A through 10H, based on the photometrically-controlled photo-activation processes illustrated in FIGS. 9A through 9D2, and internetworked with the infrastructure of the Internet; (iii) a plurality of countertop-based tissue authentication and photo-activation instrument systems 200 employing hand-held photometrically-controlled photo-activation chambers, as described in FIGS. 11A through 11E, based on the photometrically-controlled photo-activation processes illustrated in FIGS. 9A through 9D2, and internetworked with the infrastructure of the Internet; (iv) a plurality of countertop-based tissue authentication and photo-activation instrument systems 300, employing manually-actuated tissue injection guns, as described in FIGS. 12A through 12G, based on the photometrically-controlled photo-activation processes illustrated in FIGS. 9A through 9D2, and internetworked with the infrastructure of the Internet; (v) a plurality of hand-held tissue photo-activation instrument systems 400 as described in FIGS. 13A through 13F, based on the photometrically-controlled photo-activation processes illustrated in FIGS. 9A through 9D2, and internetworked with the infrastructure of the Internet; (vi) a plurality of hand-supportable mobile/wireless tissue authentication and photo-activation instrument systems 500 as described in FIGS. 14A through 14H, based on the photometrically-controlled photo-activation processes illustrated in FIGS. 9A through 9D2, and internetworked with the infrastructure of the Internet; (vii) one or more central relational database management system (RDBMS) servers 600, internetworked with the infrastructure of the Internet, along with the various instrument systems described above; (vii) a plurality of information servers 700, internetworked with the infrastructure of the Internet, for supporting administration of the tissue collection, processing and storage/banking network of the present invention; and (viii) a plurality of Web-enabled client computers 800 and internetworked with the infrastructure of the Internet, for use by users of the system and network. By virtue of this network arrangement, each of the systems indicated above supports the Internet Protocol (IP) and other higher level communication protocols (e.g. ftp, http, smb, afp, etc) providing high-speed access to the RDBMS 600 and 700, and allowing such system to read and write information files pertaining to patients, tissue donors, doctor/surgeons, and tissue specimens that have been collected, processed and banked within the network.

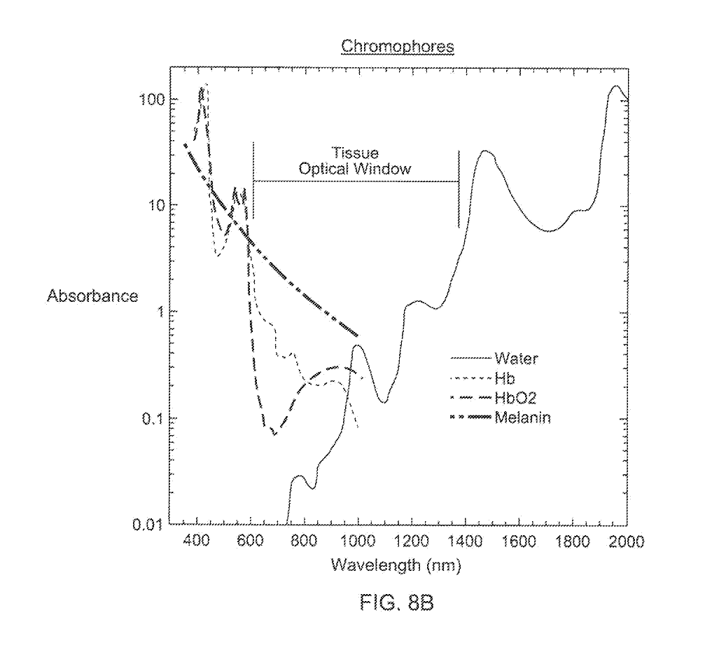

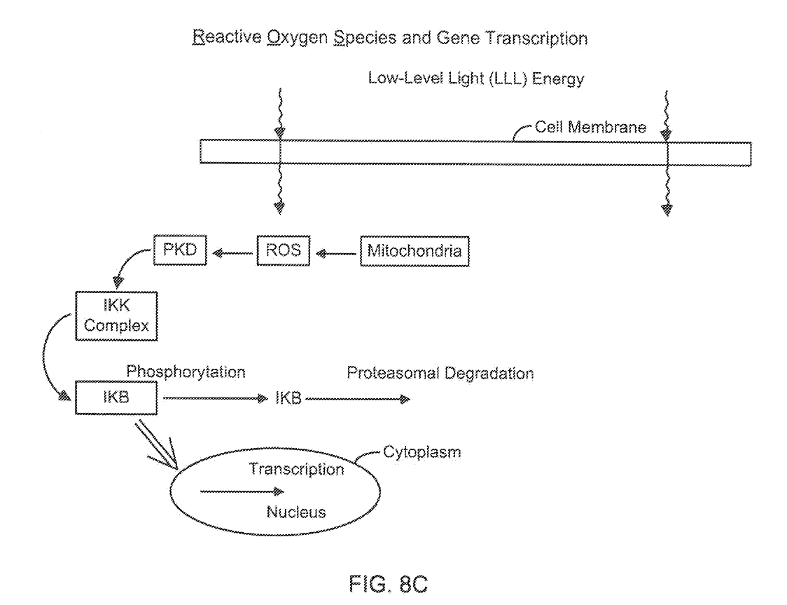

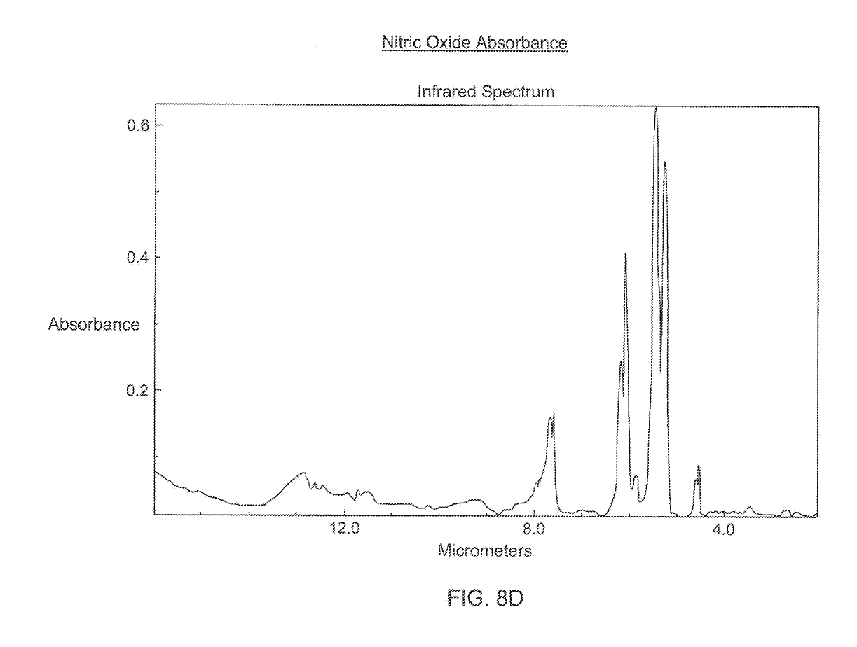

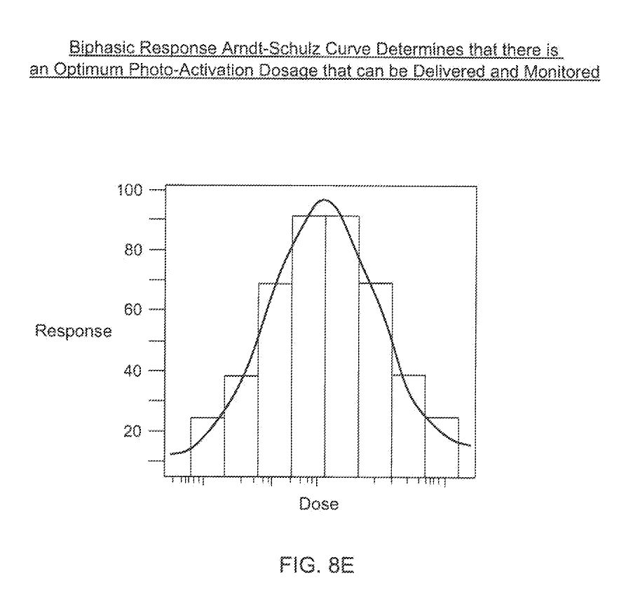

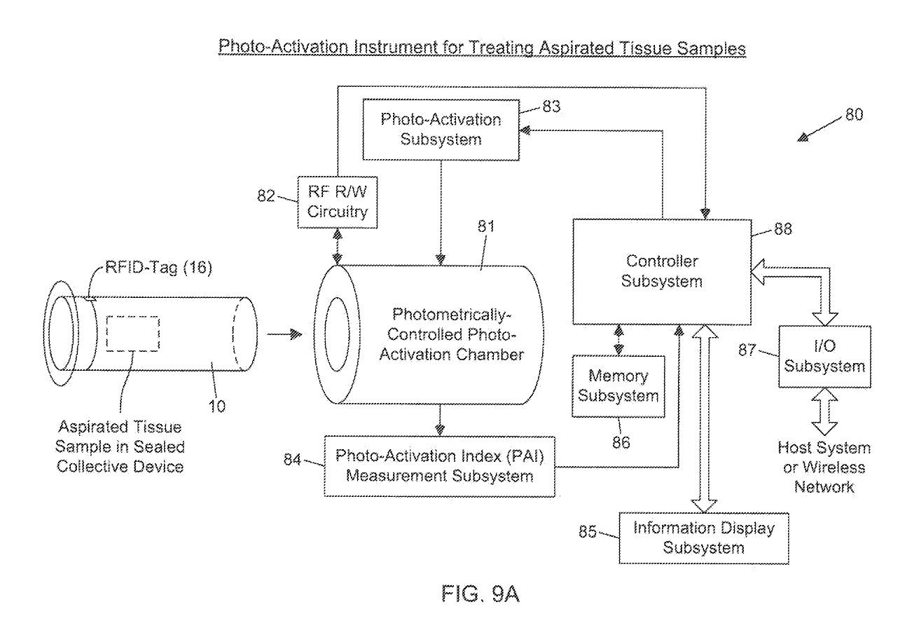

As shown, FIG. 2B illustrates the processes supported on the network of the present invention, and also the various opportunities for photo-activating collected samples of aspirated fat tissue using low level photo-active light energy, in accordance with the principles of the present invention, which will be detailed in greater detail hereinafter with reference to FIGS. 8A through 9D2.

As shown in FIG. 2B, collected fat tissue samples can be treated with non-coherent, con-collimated low level light produced from LEDs, when and as follows: after harvest; before immediate reinjection; before shipping to tissue bank; before tissue bank growth and differentiation; before shipment of banked tissue to doctor; and before doctor injects an autograft into a patient. Also, aspirated fat tissue can also be treated with multiple exposures of visible LED-based or VLD-based coherent light, or with one single exposure of coherent, collimated, laser light (capable of penetrating through the patient's skin) when and as follows: immediately after autograft in injection into patient (i.e. thru intact skin; and during subsequent patient re-visits to the doctor after the injection. Such modes of photo-activation will be described in greater detail hereafter.

Overview of Devices and Systems For Collecting and Processing Aspirated Fat Tissue According To The Present Invention

FIG. 3A shows the RFID-tagged tissue collection device of the present invention 10 having use in the different types of tissue aspiration instruments of the present invention disclosed herein, as well as in the different types of tissue processing instruments of the present invention, also disclosed herein. Also, FIGS. 3B and 3C show different views of the RFID-tagged tissue collection device 10 shown in FIG. 3A. In general, this tissue collection device 10, and devices 10′, 20, 25, 30, 40 and 60 are disclosed in Applicant's copending U.S. application Ser. No. 12/955,420 filed Nov. 29, 2010, supra, incorporated herein by reference, except for the applied of a read/write RFID tag 16 to the outer face of the tissue collection tube 11 employed in such illustrative embodiments.

FIG. 3B shows the four basic components comprising the RFID-tagged tissue collection device 10, namely: (i) a cylindrical optically transparent tissue collection tube 11 having two sets of spaced-apart micro-pores 12, 12B formed along one side of the optically transparent walls thereof; (ii) a micro-occluder 13 that slides about the outer surface of the cylindrical tissue collection tube 11 to selectively (a) occlude the micro-pores 12A, 12B when configured as shown in FIG. 4B, and (b) un-occlude the micro-pores 12A, 12B when configured as shown in FIG. 4C; (iii) a distal cap 14 for sealing off the open distal tip portion of the tissue collection tube 11; (iv) proximal cap 15 for sealing off the open proximal end opening of the tissue collection tube 11; and (v) an RFID tag 16 applied to the cylindrical tissue collection tube 11, or other suitable subcomponents of the device.

As shown in FIGS. 3A and 3B, the RFID-tagged tissue collection tube 11 according to a first illustrative embodiment is shown comprising: two sets of micro-pores 12A, 12B formed in the optically-transparent wall surfaces of the cylindrical outer tube 11; a set of flanges 17A, 17B formed at the proximal end portion similar to a standard syringe-device; an distal end portion having a standard Leur-lock fitting 18 for attachment of a cannula 21 having a matching Leur-lock fitting; and an RFID tag 16 affixed about the proximal end portion of the cylindrical collection tube 11.

In the illustrative embodiment, the RFID tag 16 can be affixed to the flange portion 17A, 17B of the cylindrical collection tube 11 at the proximal end thereof.

In FIG. 4A, the components of the RFID-tagged tissue collection device 20 of FIG. 3A are shown partially disassembled, as comprising: (i) a cylindrical optically transparent tissue collection tube 11 having micro-pores 12A, 12B formed along one side of the optically transparent walls thereof; (ii) a micro-occluder 13 that slides about the cylindrical tissue collection tube 11 to selectively occlude the micro-pores 12A, 12B when configured as shown in FIG. 4B and un-occlude the micro-pores when configured as shown in FIG. 4D; (iii) a distal cap 14 for sealing off the open distal tip portion of the tissue collection tube 11, and (iv) a plunger 19 for insertion through the proximal opening of the tissue collection tube 11 and into its interior volume to expressed a collected tissue sample out of its distal end opening 22.

When harvesting and/or injecting fat tissue using the device 20 configured in FIG. 4B, the doctor (i) inserts the plunger into the tissue collection tube (i.e. syringe-like device), (ii) rotates the micro-pore occluder 13 over the micro-pores (i.e. holes), (iii) snaps flange 13A against flange 17A, 17B so that micro-pore occluder 13 occludes the micro-pores 12A, 12B on the tissue collection tube, and (iv) then finally manually withdraws the plunger 19 to aspirate tissue, or manually pushes the plunger to eject tissue into a patient during a fat tissue injection.

When processing fat tissue collected in tissue collection 20 configured in FIG. 4C, the doctor (i) inserts the plunger 19 into the tissue collection tube (i.e. syringe-like device), (ii) rotates the micro-pore occluder 13 off the micro-pores (i.e. holes), (iii) unsnaps the flange 13A against the opposite side of flange 17A, 17B so that the micro-pore occluder 13 does not occlude the micro-pores 12A, 12B on the tissue collection tube, and (iv) then finally manually pushes the plunger 19 to express fluid from the collected tissue sample and out through the micro-pores, and thus concentrate the cellular content of the collected fat tissue sample.

Optionally, the tissue collection and processing device 20 can be reconfigured back to the state shown in FIG. 4B, so that its plunger 19 can be withdrawn within the tissue collection tube 11 and draw a quantity of saline, insulin and/or other solution into the concentrated tissue sample; then the device is reconfigured to the state shown in FIG. 4C, with the micro-pores in an un-occluded state, and then the plunger 19 is pushed into the tissue collection tube 11 to express out the fluid of the collected tissue sample, thereby washing or lavaging the same.

Collecting and Processing Aspirated Fat Tissue Using Manually-Operated Syringe-Like Tissue Collection and Processing Device of The Present Invention

FIG. 5 shows the tissue collection device of FIG. 4A equipped with a cannula 21 connected to its distal end opening via Leur-lock connector 18. Then with the micropores 12A, 12B occluded, as shown in FIG. 4B, the surgeon inserts the mounted cannula into the desired donor or treatment site, and withdraws plunger 19 to aspirate fat tissue and collect the same within the tissue collection tube 11. After concentrating and lavaging the tissue within the tissue collection tube 11, as described above, the processed fat tissue sample can be injected back into the same patient or a different compatible patient, by occluding the micro-pores 12A, 12B and then manually depressing the plunger 19 into the cylindrical interior volume of the tissue collection tube 11. Notably, the concentrated tissue sample can be photo-activated prior to reinjection into the patient using the apparatus and methods disclosed in great detail hereinbelow, for the purpose of improving graft survival, encourage differentiation and protein synthesis, and achieve higher bio-energy levels within the cells.

Collecting and Processing Aspirated Fat Tissue Using An-Line Tissue Collection Tube Chamber and Power-Assisted Tissue Aspiration Instrument

FIG. 6A shows a hand-supported power-assisted tissue aspiration instrument system 60 connected to an in-line tissue collection device 40 containing six RFID-tagged tissue collection tubes 11 of the present invention, shown in FIGS. 3E and 3F and described in detail hereinabove. FIG. 6B illustrates the construction details for the in-line tissue collection device 40 shown in FIG. 6A.

As shown in FIG. 6B, the surgeon places the distal cap 14 on each of six RFID-tagged tissue collection devices 10, and removes the proximal plug 15 and plunger 19 from their proximal opening on the tissue collection tube 11. Then the tissue collection tubes 11 are installed on mounting projection on the suction plate 41 as shown in FIG. 6B, and the collection device is reassembled as taught in great detail in copending U.S. application Ser. No. 12/955,420 filed Nov. 29, 2010, supra. Once assembled, the six-pack tissue collection tube 43 is inserted in-line with the powered tissue aspiration instrument 60 and the collection device 40, as shown in FIG. 6A, and taught in greater detail in copending U.S. application Ser. No. 12/955,420 filed Nov. 29, 2010, supra. Then the surgeon selects which tissue collection device he or she wishes to fill with aspirated fat tissue. This selection function is achieved by rotating the selector 44 using the surgeon's thumb, and rotating the selected tissue collection tube into position with the fluid passageway through the in-line collection chamber 46. The surgeon then aspirates fat tissue from the patient or donor using the powered tissue aspiration instrument 60, causing tissue to be collected within the selected tissue collection tube, while fluids are filtered out and allowed to flow through tube 48 and move towards the vacuum source 70, and the cellular content of the collected tissue sample to become concentrated. Tumescent solution can also be injected at the aspiration site and used to lavage the concentrated fat tissue sample in the tissue collection tube within the in-line tissue collection device 40. When the selected tube 11 is filled with aspirated tissue, which the surgeon can visually detect through the optically transparent tissue collection chamber walls, and optically transparent walls of the selected tissue collection device 11, then the surgeon simply selects another available tissue collection tube within the chamber by rotating the selector 44, once again using his or her thumb. Once aspirated fat tissue samples have been collected in the tissue collection chamber, the collection chamber 46 is disassembly as shown in FIG. 6B, and the tissue collection tubes 11 removed from the suction plate 41 and then quickly plugged with a proximal plug 15, and their occluder 13 rotated and snapped into position to occlude the micro-pores 12A, 12B on the tissue collection tube 11 to provide a sealed tissue collection and processing device 10, filled with an aspirate fat sample (e.g. 10 cc sample) including stem cells. Such collected tissue samples can then be further processing in accordance with the principles of the present invention, using the photo-activation techniques and apparatus disclosed and described hereinbelow in great detail, for the purpose of improving graft survival, encourage differentiation and protein synthesis, and achieve higher bio-energy levels with the cells of the tissue sample.

Overview of Photo-Activation Method and Photo-Metrically Controlled Instrument System of The Present Invention

The first law of photobiology states that for low level (power) visible light (LLL) energy to have any effect on a living biological system, the photons must be absorbed by electronic absorption bands belonging to some molecular photoacceptors, or chromophores. A chromophore is a molecule (or part of a molecule) which imparts some decided color to the compound of which it is an ingredient. Chromophore literally means, “Color lover” (L.chromo=color; L. Phore=to seek out, to have an affinity for, to love). Chromophores are generally pigmented molecules that accept photons within living tissue. When the chromophore accepts a photon, it causes a biochemical change within an atom, molecule, cell or tissue. If this change increases cellular function, it is said to have activated the tissue. If this change decreases cellular function, then it is said to have inhibited the tissue. Biomodulation occurs in both cases. Chromophores almost always occur in one of two forms: conjugated pi electron systems and metal complexes. Examples of such chromophores can be seen in chlorophyll (used by plants for photosynthesis), hemoglobin, cytochrome c oxidase (Cox), myoglobin, flavins, flavoproteins and porphyrins.

The ionizing effects of low levels of photo-active energy having photo-active wavelengths allow photon acceptors to accept an electron. This turns on the oxidation-reduction cycle of the stimulated chromophores, such as Cytochrome oxidase, hemoglobin, melanin, and serotonin. Changing the redox state of the chromophore changes the biological activity of that chromophore (e.g., hemoglobin) which changes its oxygen carrying capacity. Importantly, this photo-activation process has the potential to triple the oxygen carrying capacity of blood, instantly. In turn, direct photo-activation of cell membranes alters ion fluxes, particularly calcium, across that membrane. Changes in intracellular calcium alter the concentrations of cyclic nucleotides, causing an increase in DNA, RNA, and protein synthesis, which stimulate mitosis and cellular proliferation.

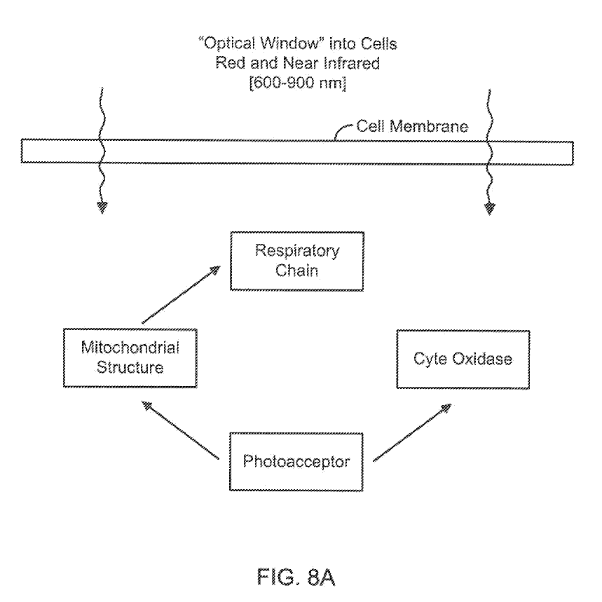

FIG. 7 illustrates the beneficial effects of low level photo-active light energy having photo-active wavelengths on cellular tissue, including proliferation, migration and adhesion, for the purpose of increasing the rate of tissue transplantation and grafting.

FIG. 8A schematically illustrates that there exists an optical window into cells over the red and near-red light band (i.e. between 600-900 nanometer wavelengths), where light energy over this photo-active band is received by photo-acceptors (i.e. chromophores) in the mitochondrial regions of the cell, and are capable of influencing the respiratory chain including the production of Cytochrome C Oxidase (Cco). While the precise mechanism by which electron transfer is coupled to proton pumping in cytochrome c oxidase is an unsolved problem in molecular bio-energetics, it is clear that the production of cyto c oxidase results in an increase in the bio-energy of the photo-activated cell.

FIG. 8B illustrates the absorbance versus wavelength response characteristics of chromophores within cellular tissue.

FIG. 8C illustrates the Reactive Oxygen Species (ROS) and gene transcription within cellular tissue.

In the reaction involving Cytochrome C Oxidase (Cco) and Nitric Oxide Release, Cytochrome C Oxidase is believed to be the primary photoacceptor for the red-NIR range in mammalian cells. Also, it is believed that nitric oxide (NO) concentrations are increased in a cell culture or in animals after exposure to low levels of photo-active energy, due to the photo release of NO from the mitochondria and Ccox. Also, as Nitric Oxide absorption mimics heme, the vitality” or activation quality of a graft should improve with increased levels of NO, correlating with induction of angiogenic factors, differentiation, level of ATP level, etc.

Also, it is believed that the release of Nitric Oxide (NO) allows the binding of oxygen species to heme.

FIG. 8D illustrates the nitric oxide (NO) versus wavelength absorbance characteristics over infrared portion of the spectrum.

FIG. 8E provides a graphical illustration of the Arndt-Schulz biphasic response curve, which indicates that there is an optimum photo-activation dosage that can be delivered and monitored to living tissue. Possible explanations for this biphasic dose response of tissue to low levels of photo-active light energy include: (1) excessive Release of Oxygen Species (ROS); (2) excessive Free Nitrogen Oxide (NO); and (3) activation of a Cytotoxic pathway. It will be helpful at this juncture to briefly discuss these three possible mechanisms for biphasic dose response.

First, current molecular and cellular mechanisms suggest that photons are absorbed by the mitochondria; they stimulate more ATP production and low levels of ROS, which then activates transcription factors, such as NF-KB, to induce many gene transcript products responsible for the beneficial effects of low levels of photo-active energy. Also, ROS is known to stimulate cellular proliferation of low levels, but inhibit proliferation and kill cells at high levels.

Second, nitric oxide (NO) is known to be photo-released from its binding sites in the respiratory chain and elsewhere. It is possible that NO release in low amounts by low dose light may be beneficial, while high levels released by high dose photo-active energy may be damaging.

Third, low levels of photo-active energy may activate transcription factors, up-regulating protective proteins which are anti-apoptotic, and generally promote cell survival. In contrast, it is entirely possible that different transcription factors and cell-signaling pathways, that promote apoptosis, could be activated after higher levels of light energy exposure.

Basic Principles of The Photo-Metrically Controlled Photo-Activation Process of The Present Invention

Based on such known principles of biology, Applicant has conceived a new way of improving graft survival, encouraging differentiation and protein synthesis, and achieving higher levels of cellular bio-energy, by “photo-activating” aspirated fat tissue specimens, in vitro, and in vivo, using photometrically-controlled delivery of low levels of photo-active light energy to such tissue components, without over dosing the same and causing deleterious effects. In accordance with principles of the present invention, photo-activation of aspirated fat tissue, and ASC components contained therein, can be carried out using (i) non-coherent non-collimated sources of light generated from visible light emitting diodes (LEDs), as well as (ii) coherent visible laser diodes (VLDs), provided that the power density of the light exposure has a sufficiently low level or intensity (i.e. photonic energy density), and the time delivery of this low levels of photo-active energy exposure to the tissue specimen is sufficient controlled to optimize the photo-activation index (PAI) of the treated tissue specimen, and avoid administering too much low levels of photo-active energy, after which the effect is deleterious—as measured by cell survival, cultured cell growth of differentiated samples, etc., predicted by the Arndt-Schulz biphasic response curve.

By using low levels of photo-active energy, form either LED and/or VLD energy sources, aspirated tissue samples and stem cells therein can be photo-activated before use as an autograft or cellular culture so as to achieve a higher energy state encouraging survival, the synthesis of cellular and angiogenic mediators, differentiation and proliferation. To measure the degree and effect of such photo-activation, and be able to optimize treatment, the preferred embodiments of the present invention using electronic photo-detectors to photometrically measure changes in the cellular and tissue absorbance at different spectral wavelengths of optical energy, over the red (i.e. 600-660 nm) spectral range and over the near-infrared (e.g. 830 nm) range, due to the effects of exposure to low levels of photo-active energy during photo-activation. It is estmated that low levels of photo-active energy at about 635 nm from a LED and/or a VLD, having a power density of about at least 5 [Joules/cm³], but not exceeding 50 [Joules/cm³], will be sufficient to “photo-activate” stem cells within an aspirated fat tissue sample, and thus increase their rate of survival, achieve more rapid proliferation, and increase cytokine (VEGF, NGF) production.

The photometrically-controlled photo-activation process of the present invention can be performed at the operating room (OR) table, or in the exam room after harvesting immediately before autograft reinjection, whether it be an un-enriched graft harvested in the same surgery, or whether it be a stem-cell bank grown graft of the patient's own cells, differentiated and grown into a 100% pure line of stem cells, activated with low levels of photo-active energy immediately before reinjection. Also collected specimens of aspirated fat tissue can be photo-activated using low levels of photo-active energy even before cells have been induced to differentiate at the skin bank, so as to encourage survival and proliferation.

Using the principles of the present invention, an autograft that has just been harvested in the operating room can be photo-activated at the time of surgery to increase proliferation and survival of the cells, and increased secretion of vascular stimulating adipokines Alternatively, the harvest autograph can be sent to a tissue bank and photo-activated over a period of several weeks allowing cultured, differentiated stem cells to grow and enrich the tissue sample.

Expectedly, the photometrically-controlled photo-activation process of the present invention can be utilized to stimulate the proliferation, growth, and differentiation of stem cells from any living organism. Using this process of photo-activation, it is expected that stem cells can be grown and differentiated into tissues or organs or structures or cell cultures for the purpose of infusion, implantation, etc, and that such growth and differentiation processes can be facilitated, enhanced, controlled or inhibited by modulating the photometrically-controlled photo-activation process. Advantageously, when stem cells are photo-activated using the principles of the present invention, there will be little or no temperature rise in the tissue sample due to low levels of photo-active energy exposure, although transient local nondestructive intracellular thermal changes may contribute via such effects as membrane changes or structured conformational changes.

There are a number of important factors that can be controlled when carrying out photo-activation.

A first factor is the frequency (i.e. wavelength-dependent) characteristics of the low levels of photo-active energy source used to carry out photo-activation of a collected fat tissue sample, and ASC contained therein. The energy content of the photons in the low levels of photo-active energy beam is dependent on the wavelength of the spectral component and Plank's constant, and shall be tuned to the band-gap response characteristics of the cellular components within the aspirated tissue sample, as discussed hereinbove, so as be absorbed and release an electron for use in reduction type reactions.

A second factor is the temporal characteristics of the low levels of photo-active energy source which determines the magnitude or intensity distribution of photonic flux when exposing an aspirated tissue sample to low levels of photo-active energy over a particular time duration. The intensity distribution of the photo-active energy beam (i.e. its photo flux) can be controlled by controlling the drive current supplied through the LEDs and/or VLDs. Drive current can be controlled to produce pulsed or continuous low levels of photo-active energy within the field of activation (FOA) over a particular duration, which might have the form of low levels of photo-active energy pulses, repeated at a particular frequency, followed by a dark or “OFF” period. Whether or not the exposed tissue sample has absorbed as many photons of a wavelength-specific low levels of photo-active energy as possible over a given treatment duration (i.e. the specimen has reached a saturation state of photo-activation) can be determined by photometrically measuring the photo-activation index (PAI) of the tissue sample during the photo-activation process. Such photometric measurements can be carried out by (i) illuminating the tissue sample with a first photo-active light (e.g. red light) energy source, (ii) measuring the intensity of first photo-active light energy transmitted through (or reflected from the sample) during the photometric measurement mode of the photo-activation process, and then repeating the same steps using a second photo-active light (e.g. IR light) energy source, and (iii) then processing the detected photometric signals from the first and second photo-active light energy sources to compute a photo-activation index (API) for the sample, based in the logarithmic ratio of the transmitted first and second photo-active light energy measurements made on the tissue sample. This process of photometric measurement and PAI computation will be described in great technical detail in FIGS. 9B though 9D2, and with respect to the various illustrative embodiments of the instrument systems of the present invention.

A third factor is the presence, absence or deficiency of any or all cofactors, enzymes, catalysts, or other building blocks of the process being photo-activated. Such material present within any given aspirated tissue sample can be thought of matter that has the capacity to absorb photonic energy from the photo-active light source, but not improve graft survival, encourage differentiation and protein synthesis, and achieve higher cellular energy levels.

Using the Photo-Metrically Controlled Photo-Activation Process to Drive Differentiation or Proliferation of Stem Cells

The photo-activation process of the present invention can control or direct the path or pathways of differentiation of stem cells, their proliferation and growth, their motility and ultimately what the stem cells produce or secrete and the specific activation or inhibition of such production. A specific set of parameters can activate or inhibit differentiation or proliferation or other activities of a stem cell. Likewise, a different set of parameters using the same wavelength of low levels of photo-active energy may have very diverse and even opposite effects. When different parameters of photo-activation are performed simultaneously, different effects may be produced. When different parameters are used serially or sequentially, the effects are also different. The selection of photo-activation wavelength is critical as is the bandwidth selected, as there may be a very narrow bandwidth for some applications—in essence because these are biologically-active spectral intervals. In general, the photo-activation process will target flavins, cytochromes, iron-sulfur complexes, quinines, heme, enzymes, and other transition metal ligand bond structures, though not limited to these cellular components.

The photonic energy received by photo acceptor molecules from sources of low level photo-active energy is sufficient to affect the chemical bonds thus ‘energizing’ the photo acceptor molecules which, in turn, transfers and may also amplify this energy signal. An ‘electron shuttle’ transports this energy to ultimately produce ATP (or inhibit) the mitochondria, thus energizing the cell (for proliferation or secretory activities for example). This bio-energization process can be broad, or very specific in the cellular response produced.

While the mechanism which establishes ‘priorities’ within living cells is not fully understood at this time, it nevertheless is possible to photo-activate the cellular components, for the purpose of promoting proliferation and differentiation of the stem cell population in an collected specimen of aspirated fat tissue.

It is believed that photo-activation parameters can function much like a “morse code” of sorts to communicate specific instructions to stem cells. This has enormous potential, in practical terms, such as guiding or directing the type of cells, tissues or organs that stem cells develop or differentiate into, as well as stimulating, enhancing or accelerating their growth, or keeping stem cells undifferentiated.

It is known that the spectral energy having a 635 nm wavelength falls within the wavelength spectrum of all biological chromophores, in both man and animals. Also, it is known that different chromophores have peak activation somewhere between 600 nm and 720 nm. Thus, each chromophore can still be photo-activated using a wider wavelength spectrum so long as spectral component having a 635 nm wavelength falls within the wavelength spectrum, thus avoiding the need to utilize multiple colors of low levels of photo-active energy to photo-activate the different chromophores in the human body. In short, over the visible band of the electro-magnetic spectrum, a single wavelength (i.e. 635 nm) should have the potential to photo-activate every biologically photosensitive receptor in the human body.

Three specific and unique ways are proposed below for the way the 635 nm wavelength of low levels of photo-active energy can photo-active a specimen of aspirated fat tissue in accordance with the principles of the present invention.

According to the first proposed mechanism, within the cell, the signal is transduced and amplified by a photon acceptor (chromophore). When a chromophore first absorbs light, electronically excited states are stimulated, primary molecular processes are initiated which lead to measurable biological effects. These photobiological effects are mediated through a secondary biochemical reaction, photosignal transduction cascade, or intracellular signaling which amplifies the biological response.

According to the second proposed mechanism, ionizing effects of low levels of photo-active energy allow photon acceptors to accept an electron. This turns on the oxidation-reduction cycle of the stimulated chromophores such as Cytochrome oxidase, hemoglobin, melanin, and serotonin. Changing the redox state of the chromophore changes the biological activity of that chromophore e.g., hemoglobin changes its oxygen carrying capacity. This has the potential to triple the oxygen carrying capacity of blood instantly.

According to the third proposed mechanism, when photon energy breaks a chemical bond, changes occur in the allosteric proteins in cell membranes (cell, mitochondrial, nuclear) and monovalent and divalent fluxes activate cell metabolism and intracellular enzymes directly. Direct activation of cell membranes alters ion fluxes, particularly calcium, across that membrane. Changes in intracellular calcium alter the concentrations of cyclic nucleotides, causing an increase in DNA, RNA, and protein synthesis, which stimulate mitosis and cellular proliferation.

In order to photometrically control the photo-activation process of the present invention, a far infrared wavelength of light (e.g. 830 nm) is required, in conjunction with a red wavelength component (e.g. 635 nm), to carry out the “photometric mode” of this process. Also, it would be advantageous if the 830 nm spectral component would have photo-active effects, in not photo-active effects, then at least beneficial effects during the “photo-activation mode” of the photo-activation process, as this would encourage this source of energy to be used during the photo-activation process.

Surprising, the 830 nm (near infrared) wavelength is absorbed in the cellular membrane, rather than in cellular organelles, which is the target of the photo-activation process of the present invention. Such wavelength absorption leads to accelerated fibroblast-myofibroblast transformation and mast cell degranulation. In addition, the 830 nm wavelength enhances chemotaxis and phagocytic activity of leucocytes and macrophages through cellular stimulation by this wavelength. Such photo-activated by-products have a number of beneficial effects. In particular, accelerated fibroblast-myofibroblast transformation results in an intermediate autograft which is beneficial. Accelerated mast cell degranulation may encourage some neovascularization which is beneficial. Enhancement of leukocytes reduces infection which is also beneficial. In short, exposing aspirated tissue samples to the 830 nm wavelength of low levels of photo-active energy during photo-activation, and during photometric measurement, is beneficial as it helps cellular membranes increase Ca levels and improve cellular adhesion. Thus, the 830 nm wavelength is an excellent source of far infrared light during both photometric and photo-activation modes of the photo-activation process of the present invention, which will be specified in greater technical detail hereinbelow.

Specification of the Photometrically-Controlled Photo-Activation Apparatus and Process of The Present Invention

FIG. 9A shows a generalized model of the photometrically-controlled photo-activation instrument system of the present invention 80, comprising the following components: (i) a photometrically-controlled photo-activation chamber 81 adapted to receive at least one RFID-tagged sealed tissue collection device 10 for photo-activation treatment using a photometrically-controlled photo-activation process supported by the instrument; (ii) RFID tag read/write subsystem 82 for reading from and writing to the RFID tag 16 on the tissue collection device 10; (iii) a photo-activation (illumination) subsystem 83 for illuminating the aspirated tissue sample in the tissue collection device 85; (iv) a real-time photo-activation index (PAI) measurement subsystem for measuring the photo-activation index (PAI) of the collected tissue sample during and after photo-activation operations, and determining whether or not the rate of changes in PAI are sufficient to terminate the photo-activation of the aspirated fat tissue specimen; (v) an information display subsystem 85 for displaying information to the doctor and other medical personnel during instrument operation; (vi) a memory subsystem (e.g. DRAM, SRAM, FLASH, solid-state hard-drive, etc) 86 for storing and retrieving information regarding tissue samples collected and processed by the instrument system 80 or associated with a tissue banking system; and (vii) an input/output (I/O) subsystem 87 for interfacing the instrument system with one or more host systems and/or wired and/or wireless data communication networks; and (vii) a control subsystem 88 for controllably the operation of the subsystems described above.

FIG. 9B provides a prophetic example of the photo-activation response characteristics of an in vitro aspirated tissue sample, showing that the photo-activation index (AI) of an aspirated fat tissue specimen increases with low levels of photo-active light energy exposure, and then decreases after a particular amount of such light energy exposure.

The photo-activation system 80 shown in FIG. 9A is capable of photo-activating a collected sample (i.e. specimen) of fat tissue contained in self-contained tissue collection and processing device 10 during periodically repeating photo-activation and photometric modes of operation, preferably having a duty ratio of about 9 to 1. During its photometric mode of operation, the system 80 exposes the collected fat tissue sample to low levels of red and infrared light energy while contained within the tissue collection and processing device, so as to photometrically determine the photo-activation index (PAI) of the specimen. Then, during the photo-activation mode of operation, the system exposes the collected fat tissue sample to low levels of photo-active energy for a predetermined time period. This process of switching between photometric and photo-activation modes of operation repeats periodically until the rate of change of the PAI is essentially zero, indicating that the collected fat tissue specimen is fully photo-activated, having improved capacity to bind free oxygen species, and improving the vitality thereof during autografting operations.

In general, there are many different ways of reducing to practice, the photometrically-controlled photo-activation system 80 and process of the present invention. For purposes of illustration, two different methods of photometrically-controlled photo-activation are described in FIGS. 9C1 and 9C2, and 9D1 and 9D2, respectively.

FIGS. 9C1 and 9C2 describe the primary steps of a first illustrative in vitro method of photo-activating a collected sample of aspirated fat tissue using photometric (i.e. nitric-oximetry) feedback principles supported by the instrument system 80 shown in FIG. 9A.

As indicated at Block A in FIG. 9C1, the first step of the process involves powering up the photo-activation instrument 80, and loading into memory 86, Photo-Activation Index (PAI) Thresholds empirically determined for particular tissue samples. PAI Thresholds can be experimentally determined for any given system design.

As indicated at Block B in FIG. 9C1, the second step of the process involves collecting an aspirated tissue sample in a sealed RFID-tagged tissue collection device, and then sealing the tissue collection tube for treatment.

As indicated at Block C in FIG. 9C1, the third step of the process involves loading the sealed tissue collection device 10 inside a photometrically-controlled photo-activation chamber of the photo-activation instrument 80.

As indicated at Block D in FIG. 9C1, the fourth step of the process involves measuring and recording the initial Photo-Activation Index (PAI) of the collected tissue sample in the chamber 81.

As indicated at Block E in FIG. 9C2, the fifth step of the process involves determining whether or not the Photo-Activation Index of the measured tissue sample is equal to or greater than the Photo-Activation Index Threshold.

If at Block E in FIG. 9C2, it is determined that the PAI equals the PAI is equal to or greater than the PAI Threshold, then the process involves at Block F recording the measured Photo-Activation Index on the RFID tag of the tissue collection device, 10 and then at Block G, the process involves removing the tissue collection device from the chamber, for subsequent processing, reinjection or banking operations.

If at Block E in FIG. 9C2, it is determined that the PAI is not equal to the PAI Threshold, then the process involves at Block H photo-activating the collected tissue sample within the photometrically-controlled photo-activation chamber 81.

As indicated at Block I in FIG. 9C2, the process then involves measuring and recording the Photo-Activation Index of the collected tissue sample on the RFID tag 16.

Then, at Block J in FIG. 9C2, the process determines whether or not the Photo-Activation Index of the measured tissue sample is equal to or greater than the Photo-Activation Index Threshold, and if so then proceeds to Block F, as shown. If the measured PAI is not equal to or greater than the PAI

Threshold for the tissue specimen, then the process returns to Block H and continues to undergo photo-activation.

The photometrically-controlled photo-activation process described above continues within its control loops illustrated in FIG. 9C2 until the tissue specimen (i.e. sample) is sufficiently photo-activated in accordance with the principles of the present invention.

FIGS. 9D1 and 9D2 describes the primary steps of a second illustrative in vitro method of photo-activating a collected sample of aspirated fat tissue using photometric (i.e. nitric-oximetry) feedback principles supported by the instrument system 80 shown in FIG. 9A.

As indicated at Block A in FIG. 9D1, the first step of the process involves powering up the photo-activation instrument, and loading into memory 86, the duration of the photo-activation treatment mode or cycle (e.g. X [seconds]) and the Photo-Activation Index (PAI) Test Threshold (a dimensionless figure).

As indicated at Block B in FIG. 9D1, the second step of the process involves collecting an aspirated tissue sample in a sealed RFID-tagged tissue collection device 10, and then sealing the tissue collection tube for treatment.

As indicated at Block C in FIG. 9D1, the third step of the process involves loading the sealed tissue collection device 10 inside a photometrically-controlled photo-activation chamber 81 of the photo-activation instrument.

As indicated at Block D in FIG. 9D1, the fourth step of the process involves at measuring and recording, at time T(t), the initial Photo-Activation Index (PAI) of the collected tissue sample in the photometrically-controlled photo-activation chamber of the instrument system 80.

As indicated at Block E in FIG. 9D2, the fifth step of the process involves photo-activating the collected tissue sample within the photometrically-controlled photo-activation chamber 81.

As indicated Block F in FIG. 9D2, the process involves photometrically measuring and recording the initial Photo-Activation Index of the collected tissue sample in the photometrically-controlled photo-activation chamber.

At Block G in FIG. 9D2, the process determines whether or not the measured Photo-Activation Index (PAI) Difference (i.e. ΔPAI=PAI(t+X)−PAI(t)) has increased, decreased or remained essentially zero (i.e. close to the ΔPAI Test Threshold).

As indicated at Block G, if ΔPAI has increased, then the process returns to Block E and continues another photo-activation cycle. If ΔPAI has decreased, or retains essentially zero (i.e. close to the APAI Test Threshold), then the process proceeds to Block H and records the last measured Photo-Activation Index (PAI) of the collected tissue sample, on the RFID tag 16 of its tissue collection device 10 loaded within the photometrically-controlled photo-activation chamber 81.

Then, at Block I in FIG. 9D2, the process involves removing the tissue collection device from the chamber 81, for subsequent processing, reinjection and/or banking operations.

The photometrically-controlled photo-activation process described above continues within its control loops illustrated in FIG. 9D2 until the tissue specimen (i.e. sample) is sufficiently photo-activated in accordance with the principles of the present invention.

When practicing the photo-activation process of the present invention, before and after treatment, it is recommended using LED-based low levels of photo-active energy, rather VLD sources, despite the fact that VLD sources are capable of penetrating intact skin because of coherency and collimated properties of laser light sources. However, to avoid generation of non-photo-active heat energy within the tissue specimen, it is preferable to treat patient tissue using only the 635 mm wavelength to avoid the generation of heat energy, and ensure that all chromophores are photo-activated within an aspirated tissue sample. For this purpose, 635 nm wavelength LED sources are recommended when constructing the photo-activation illumination subsystem. Such LED-based low levels of photo-active energy sources can be used to photo-activate in vitro tissue specimen before injection into a patient, as well as after the tissue has been injected into the patient, to treat the area of injection afterwards to maintain the photo-activation state of the transplanted or grafted tissue, and assure an optimal result.

When using a 635 nm VLD is used to implement the photo-activation illumination subsystem 83 the power-density of low levels of photo-active energy field should still reside within the above indicated power density limits to avoid over photo-activating aspirated tissue with deleterious effects. When designed properly, the VLD-based photo-activation instrument should function quite similar to a LED-based photo-activation instrument, with the exception being that fewer VLDs will be required to meet the low levels of photo-active energy requirements of the system under design. Also, the use of LEDs should reduce manufacturing costs as well.

The photo-activation instrument systems 100, 200, 300 and 500 described in FIGS. 10A through 12G and 14A through 14F have been particularly designed for treating in vitro fat aspirate specimens collected in tissue collection devices, using low levels of photo-active energy produced from an array of red and near infrared LED sources, both before injection and before plating out and culturing at skin bank, so as to activate the organelles and membranes with dual 635 nm and 830 nm wavelengths of low levels of photo-active energy, respectively.

In contrast, the photo-activation instrument system 400 described in FIGS. 13A through 13F has been designed for treating the post-injected area, for the same purpose, namely to activate the organelles and chromphores and membranes to encourage secretion of cytokines and proliferation using either (i) low levels of photo-active energy from LED sources for one or multiple treatments, or (ii) alternatively using a collimated and coherent light from VLD sources, so as to allow more penetration into the subcutaneous tissue and dermis. Here single the single 630 nm wavelength of low levels of photo-active light energy is safer to avoid the heat generating propensity of the near IR (830 nm) wavelength light.

Notably, a primary advantage when using LED-based low levels of photo-active energy sources is that such devices are classified as Class IIIB light sources, rather than Class IIIA sources, thus obviating FDA approval, allowing the delegation of paramedical staff, while lowering the probability of overdosing and harming the patient.

Tissue Photo-Activation Instrument System of the First Illustrative Embodiment of The Present Invention

FIG. 10A shows a first illustrative embodiment of the tissue authentication and photo-activation instrument system of present invention 100 having the form of countertop supportable instrument system with its photometrically-controlled photo-activation chamber 101 installed within the housing 102 of the console unit 103 having controls 104, an LCD touch-screen display panel 105, and display panel 106. This system is capable of authenticating and photo-activating a collected fat tissue sample contained in a sealed tissue collection device 10 inserted within its photometrically-controlled photo-activation chamber 101. During photo-activation, the specimen of fat tissue undergoes photo-activation treatment by low levels of photo-active light energy having photo-active wavelengths of about 635 and 830 nanometers, emitted from arrays of visible light emitting diodes (LEDs) and/or visible laser diodes (VLDs) surrounding the sealed tissue collection tube 10, while the photo-activation index (PAI) of the fat tissue sample is photometrically-measured between periodically-alternating modes of photo-activation, to ensure that the fat tissue sample is optimally photo-activated, and over-activation is avoided.

As shown in FIG. 10B, the tissue authentication and photo-activation instrument system 100 comprises a number of subcomponents, namely: a plurality of photo-detection (i.e. photo-diode) arrays 110A through 110D, with good response characteristics over the 635 nm and 830 nm regions, and whose analog output signals are provided to a band of pre-amplifier circuits 111, band-pass filters 112, and analog/digital (A/D) converters 113, interfaced with the system bus 114 of the system; a plurality of illumination arrays (e.g. formed from 635 nm and 830 nm LEDs and/or VLDs) 115A through 115D, driven by drive currents supplied by drive circuits 116, which are controlled by digital/analog (D/A) converters 117, interfaced with the system bus 114, as shown; a piezo-electric transducer 118, for generating vibrations that agitate the tissue sample in a tissue collection tube 10 during photo-activation, driven by the output signals from a D/A converter 119 which is interfaced with the system bus 114; reference signal generating circuits 120; a clock 121 for generating clock timing signals used by the system; an AC/DC power adapter 122 interfaced with a power distribution circuit 123 supplied with backup power from a backup battery 124, and supplying electrical power to all electrical power consuming components within the system; a micro-processor 125, interfaced with the system bus 114 and supported by a memory subsystem including SDRAM 126, EPROM 127 and FLASH RAM 128, and FLASH drive 129; a communication interface 130 interfaced with the system bus 114, and supporting wireless WIFI communication protocols, Ethernet, USB, Firewire, Serial and other networking protocols; an input/output (I/O) interface 131 for interfacing a LCD touchscreen display panel 105, membrane keypad 105B, LCD display panel 106 and audio transducer 133; and RFID tag reading/writing subsystem 135 interfaced with the system bus 114, including an antenna, for communicating with diverse types of RFID R/W tags 16 applied to the tissue collection and processing devices 10 of the present invention, and reading from and writing to memory onboard these RFID tags 16 during the photometrically-controlled photo-activation process of the present invention. Also, a hinged cover 160 provided to close off the tissue specimen in device 10 from ambient light during photo-activation and photo-metric operations performed by the system 100.

As shown in FIG. 10D, a sealed RFID tagged tissue collection and processing device 10 is inserted within the lensed barrel insert 140 within the chamber 101, allowing the RFID tag reading/writing subsystem 135 to read from and write to the RFID tag during photometrically-controlled photo-activation operations, described in detail hereinabove.

As shown in FIGS. 10A, 10G1 and 10G2, a plurality of LEDs (or VLDs) 115 and a plurality of photo-diodes 110 are mounted though mounting holes 148 formed in the outer optically-transparent cylindrical chamber tube 141 which can be flanged on opposing ends for mounting and/or support purposes. Within this optically-transparent cylindrical chamber tube 141, the lensed barrel insert 140 is slidably mounted and has appropriately designed lenses 145 arranged on from of each LED (or VLD) 115 and photo-diode 110 mounted within the photo-activation photometric chamber 101 of the present invention.

As shown in FIGS. 10E1 and 10E2, the lensed barrel insert 140 is preferably made from a high-grade optically transparent plastic so that the lenses 145 formed within this structure have high clarity for focusing the light rays produced from the LEDs (and/or VLDs) 115 mounted within the chamber tube 141 and lensed barrel insert 140, so that they expose regions of the collected tissue sample, within the sealed tissue collection and processing device 10, during the photo-activation mode, and within the field of view (FOV) of the respective photo-diodes during the photometric mode, as shown in FIGS. 10G1 and 10G2. To ensure that maximum amount of light rays are exposed to and absorbed within the sealed tissue sample, the lensed barrel insert 140 is provided within a mirrored surface (i.e. deposited light reflective coating) 146 on all interior surfaces of lensed barrel insert 140 facing the tissue specimen, other than surfaces where the lensed LEDs and lensed photo-diodes are mounted. This light reflective coating 146 should be tuned to reflect all wavelengths of light over the working bandwidth of the photometrically-controlled photo-activation chamber 101, during both the photo-activation and photometric modes of operation, so as to ensure (i) optimal absorption of low levels of photo-active energy during the photo-activation mode, and (ii) sufficient detected signal strength from both the red (630 nm) and near IR (830 nm) signals transmitted into the tissue sample during the photometric mode of operation of the system when Photo-Activation Index (PAI) measurements are being automatically performed and recorded by the instrument system.

As shown in FIG. 10D, the photometrically-controlled photo-activation chamber has its piezo-electric transducer 118 mounted in direct contact with the bottom distal cap portion 114 of the sealed tissue collection and processing device 10 of the present invention, so as to cause the tissue sample in the tissue collection tube, to vibrate and uniformly mix during photo-activation operations supported within the photo-activation instrument system of FIG. 10B, and optionally, during the photometric mode of operation when PAI measurements are being performed by the system 100.

As a first option, the piezo-electric transducer 118 can be replaced by a vibrator motor (i.e. a motor having an asymmetrically weighted fly wheel) so that low frequency agitation of the tissue specimen occurs when exposed to the low levels of photo-active energy during the photo-activation mode of the system. If audible sonic vibration is to be used for tissue agitation, then it is suggested that the key of E (i.e. 329 Hz) is an ideal frequency of agitation, although it is understood that other frequencies will work successfully for the purpose at hand.

As a second option, the piezo-electric transducer 118 can be replaced by a source of ultrasonic vibrational energy which has particular value when the tissue aspirate is to be cultured for replication of line differentiation, as ultrasonic vibrations will tend to free the cells from remnant adipose stromal tissue.

As shown in FIG. 10B, the RFID tag reading/writing subassembly 135 is installed within console 103, preferably proximate to the proximal end of the chamber 101, so that the subsystem 135 is able to read data from and write data to the RFID tag 16 on the tissue collection and processing device 10 loaded therein during photo-activation operations, via electromagnetic communication via RF antennas in the RFID tag 16 and RFID reading/writing subsystem 135 in a manner known in the art.

As shown in FIGS. 10C, a read-write RFID tag 16 is incorporated into or on the tissue collection and processing device 10 to record at least the following information items: date tissue was harvested; photo-activation and/or any optional procedures and or lavage performed; physician harvesting; patient name and social security; date grown; and date cryopreserved. Preferably, the RFID tags 16 will both electronically scannable using RFID tag reading/writing subsystem 135, as well as display particular fields of graphical information on an electronic ink display label 16B forming an integral part of the RFID tag 16, as taught in U.S. Pat. No. 7,913,908 to Gelbman, incorporated herein by reference, in its entirety. In addition, a printed barcode symbol can be applied to the RFID tag, or along side thereof, readable by an optical bar code symbol reader. The bar code symbol can be constructed using a high-density 2D symbology such PDF 417, or an suitable 2D datamatrix symbology known in the automatic identification (AUTOID) art.

During every photo-activation treatment, the onboard memory of the RFID tag 16 on the tissue collection device 10 can be written to reflect the PAI of the tissue sample at the instant in time of the writing operation. At indicated times, information recorded on the RFID tag 16 of any given tissue collection device 10 be transmitted to the central RDBMS 600 by the instrument system 100 which is internetworked with the central RDBMS 600 and other network servers 700, and client machines 800. Cultured lines of replicated stem cells, or differentiated lines such as adipocytes, will be placed in RFID tagged tissue collection containers 10 of the present invention. These RFID tags 16 will record information tracing the harvesting of the tissue sample, and all subsequent photo-activation treatments, and/or agents it has received to promote differentiation. The tissue labeling and cataloguing system of the present invention will allow a physician treating a moribund patient with a post-myocardial infract akinetic ventrical to quickly determine which laboratory has cultured myocytes that may be used to restore ventricular function to his or her patient. Similarly, physicians treating patients with spinal injuries can access the central RDBMS 600 and determine which laboratory has cultured neuronal cells that may be used to repair spinal injuries.

To protect the patient and its information, a simple PGP key system can be used to encrypt the data recorded on the RFID tag 16 on tissue collection devices 10 of the present invention. The data would be encrypted with the key of provider or facility. Any authorized user would have to have the provider or facility key to decrypt the information on the RFID tag. UPN registration for licensed physicians would incorporate this PGP key into the central RDBMS 600, and all licensed physicians would be provided access to the RDBMS 600. JCAAH would incorporate a facility PGP key into the central RDBMS 600, and all accredited hospital or Article 28 Ambulatory Surgery facilities to be provided access to the RDBMS 600 on the tissue banking network of the present invention.

In addition, each patient could be assigned an intelligent bracelet, intelligent card 150, or RFID tag 16, containing data encrypted specifying the primary physician's key and where banked patient tissue is stored and what cell lines are available for the patient (e.g. in the event of a heart attack, neural injury, cartilage replacement, etc.). That key could be obtained by an emergency room (ER), or EMS from UPN, to provide all licensed EMS or facilities to gain access to the central RDBMS 600 on the tissue banking network of the present invention.

Also, within the central RDBMS 600, there would be multiple levels of authorized access and data encryption to sensitive data such as, for example, a patient's H.I.V. status, psychiatric records, etc. Sensitive data would require a key from the patient, his/her representative, a court, or a treating physician or at least require an entry logging access into such records of the central RDBMS 600 on the tissue banking network.

Techniques for Photometrically-Controlling the Photo-Activation Process of The Present Invention

As explained hereinabove, delivering low levels of photo-active energy doses over the 635 nm and 830 nm wavelengths can be expected to affect chromophores within the contained tissue specimen. Notably, chromophores are photoacceptors with peak acceptance at these specific wavelengths, and which at some point decrease their affinity for photons and perhaps affect the ratio of those affinities as well. Thus, it is an object of the present invention to photometrically measure changes in those photon-affinities by the following process: (i) during a first measuring interval, transmitting a first photo-active light (e.g. red 635 nm wavelength light) energy from a first array of LEDs into the tissue specimen, and measuring the transmitted or reflected response from the tissue specimen using one or more photo-detectors; (ii) during a second measuring interval, transmitting a second photo-active light energy (e.g. IR 830 nm wavelength light) from a second array of LEDs into the tissue specimen, and measuring the transmitted or reflected response from the tissue specimen using one or more photo-detectors; and (iii) processing the measured intensities of the transmitted Red and IR signals so as to compute a Photo-Activation Index (PAI) that provides a logarithmic ratio of RED and IR photon affinities in the tissue specimen, before, during or after photo-activation treatment in accordance with the principles of the present invention Such PAI measurements may be taken before, after, and at interruptions of a continuous levels of photo-active light energy exposure, or between pulses of a pulsed levels of photo-active light energy exposure, during the photo-activation process of the present invention. This methodology is used to determine maximal photo-activation treatment range before a biphasic low levels of photo-active energy response becomes noxious in the tissue specimen.

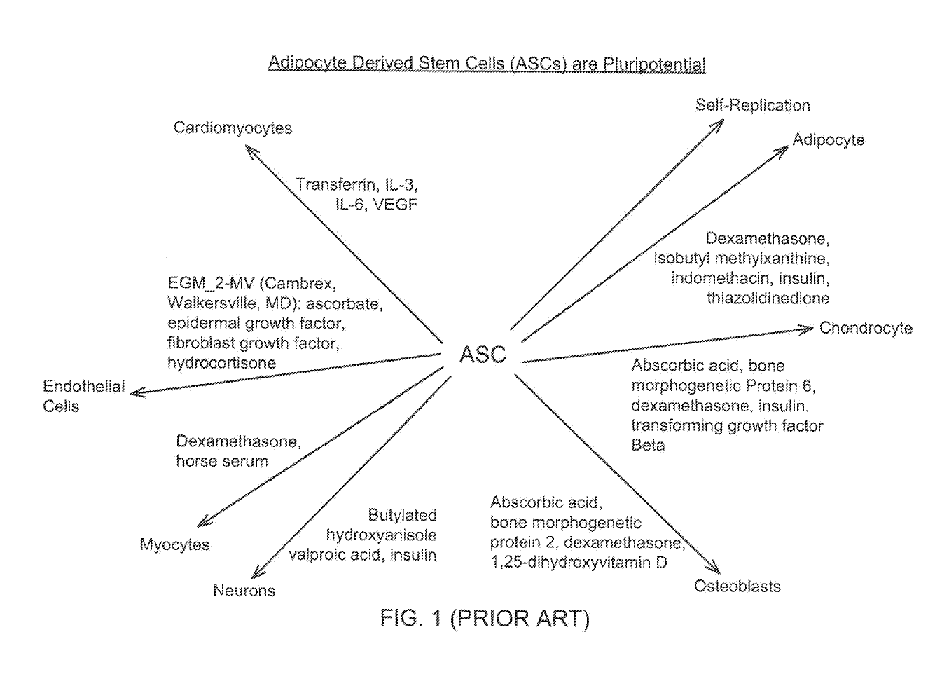

FIG. 10F describes a formula that can be used to compute the Photo-Activation Index (PAI) of an aspirated tissue sample at any testing interval within the instrument system 100 of FIG. 10A having the photometrically-controlled photo-activation chamber design specified in FIGS. 1G1 and 10G2.

FIG. 10H illustrates the timing of the photo-activation periods relative to the photometric periods of the instrument system 100 of FIG. 10A. As shown, in the illustrative embodiment, the duty cycle of the photo-activation period is about nine (9) times longer than the duty cycle of the photometric period when the Photo-Activation Index (PAI) of the tissue sample is measured and then used to control the photo-activation process of the present invention, to achieve optimal treatment without overdosing and resulting in deleterious effects.

During each photo-activation period, each 635 nm LED and 830 nm LED is driven to achieve LLE energy exposure upon an aspirated fat tissue sample, having a maximum volumetric power density that does not exceed a predetermined range for aspirated fat tissue (e.g. 5-60 Joules/Volume). During this time, the photo-diodes remain idle and no photometric measurements are made or recorded. Preferably, time duration of each photo-activation period (i.e. interval) is estimated to be about 30 [seconds], followed by a relatively short photometric period, then followed by another photo-activation period, repeating in a cyclical manner in accordance with the photometrically-controlled photo-activation process of the present invention, described in FIGS. 9C1 and 9C2 or 9D1 and 9D2.

During each photometric period, the following operations are automatically performed: (i) driving the RED LEDs indicated in FIGS. 10G1 and 10G2 and measuring and recording intensity response of the tissue sample within the FOV of the respective photo-diode; (ii) driving the IR LEDs indicated in FIGS. 10G1 and 10G2 and measuring and recording intensity response of the tissue sample within the FOV of the respective photo-diode; and (iii) processing the measured intensities of RED and IR signals according to the PAI formula set forth above to compute a Photo-Activation Index (PM) that provides a logarithmic ratio of RED and IR photon affinities in the tissue specimen, in accordance with the principles of the present invention.

This PAI value is recorded in memory 127, 128, and the microprocessor 125 carries out the photometrically-controlled photo-activation process programmed into system memory. When the optimal PAI has been attained, expectedly taking anywhere between 30 seconds and 5 minutes, depending on the photo-activation state of the aspirated tissue specimen when inserted into the photometrically-controlled photo-activation chamber 101.

Tissue Photo-Activation Instrument System of the Second Illustrative Embodiment of The Present Invention

FIG. 11A shows a second illustrative embodiment of the tissue authentication and photo-activation instrument system of present invention 200 having the form of countertop supportable instrument system with its photometrically-controlled photo-activation chamber 101′ embodied within a hand-supportable unit 202 having a flexible cable 203 that establishes an electrically interface with a console unit 204 having a housing 205 and controls 206, an LCD touch-screen display panel 207, and display panel 208. System 200 is capable of authenticating and photo-activating a collected fat tissue sample contained in a sealed tissue collection device 10 inserted within the photometrically-controlled photo-activation chamber 101′ of the hand-held unit which is similar to chamber 101, but shorter in length, based on design considerations. During photo-activation, the specimen of fat tissue undergoes photo-activation treatment by low levels of photo-active light energy having photo-active wavelengths of about 635 and 830 nanometers, emitted from arrays of visible light emitting diodes (LEDs) and/or visible laser diodes (VLDs) surrounding the sealed 10 [cc] tissue collection tube 10, while the photo-activation index (PAI) of the fat tissue sample is photometrically-measured between periodically-alternating modes of photo-activation, to ensure that the fat tissue sample is optimally photo-activated, and over-activation is avoided.

As shown in FIG. 11 B, the tissue authentication and photo-activation instrument system 200 comprises a number of subcomponents, namely: a plurality of photo-detection (i.e. photo-diode) arrays 110A through 110D, with good response characteristics over the 635 nm and 830 nm regions, and whose analog output signals are provided to a band of pre-amplifier circuits 111, band-pass filters 112, and analog/digital (A/D) converters 113, interfaced with the system bus 114 of the system; a plurality of illumination arrays (e.g. formed from 635 nm and 830 nm LEDs and/or VLDs) 115A through 115D, driven by drive currents supplied by drive circuits 116, which are controlled by digital/analog (D/A) converters 117, interfaced with the system bus 114, as shown; a piezo-electric transducer 118 for generating vibrations that agitate the tissue sample in a tissue collection tube 10 during photo-activation, driven by the output signals from a D/A converter 119 which is interfaced with the system bus 114; reference signal generating circuits 120; a clock 121 for generating clock timing signals used by the system; an AC/DC power adapter 122 interfaced with a power distribution circuit 123, supplied with backup power from a backup battery 124, and supplying electrical power to all electrical power consuming components within the system; a micro-processor 125 interfaced with the system bus 114 and supported by a memory subsystem including SDRAM 126, EPROM 127, FLASH RAM 128, and FLASH drive 129; a communication interface 130 interfaced with the system bus 114, and supporting wireless WIFI communication protocols, Ethernet, USB, Firewire, Serial and other networking protocols; an input/output (I/O) interface 131 for interfacing a LCD touchscreen display panel 105, membrane keypad 105B LCD display panel 106 and audio transducer 133; and RFID tag reading/writing subsystem 135 interfaced with the system bus 114, including an antenna, for communicating with diverse types of RFID R/W tags 16 applied to the tissue collection and processing devices 10 of the present invention, and reading from and writing to memory onboard these RFID tags 16 during the photometrically-controlled photo-activation process of the present invention.

FIG. 11 C provides a perspective view of the hand-held unit 201 embodying the photometrically-controlled photo-activation chamber 101′ in its compact hand-held housing.

As shown in FIGS. 11B and 11C, the RFID tag reading/writing subassembly 135 is installed within hand-supportable housing 210, preferably proximate to the proximal end of the chamber 101′, so that the subsystem is able to read data from and write data to the RFID tag 16 on the tissue collection and processing device 10 loaded therein during photo-activation operations, via electromagnetic communication via RF antennas in the RFID tag 16 and RFID reading/writing subsystem 135 in a manner known in the art.

As shown in FIG. 11C, a sealed RFID tagged tissue collection and processing device 10 is inserted within the chamber 101′, allowing the RFID tag reading/writing subsystem 135 to read from and write to the RFID tag during photometrically-controlled photo-activation operations, described in detail hereinabove. In all important respects, the photometrically-controlled photo-activation chamber 101′ shown in FIGS. 11C and 11E1 and 11E2 is similar to the photometrically-controlled photo-activation chamber 101 shown in FIGS. 10D, and 10G1 and 10G2, except the length of the chamber is shorter, and employs fewer RED and IR LEDs. However, it is understood that the photometrically-controlled photo-activation chamber 101′ employed in the hand-held device 202 can be extended to treat longer length RFID tagged tissue collection and processing devices 10 according to the present invention, as applications may require. Also, the inner diameter of the photometrically-controlled photo-activation chamber 101′ can be enlarged or made smaller to adapted to the outer diameter of the RFID tagged tissue collection and processing devices. Also, it is understood that the photometrically-controlled photo-activation chamber 101′ can be provided with a piezo-electric transducer 118 mounted in direct contact with the bottom distal cap portion 114 of the sealed tissue collection and processing device 10, to vibrate and uniformly mix during photo-activation operations supported within the photo-activation instrument system of FIG. 11B, and optionally, during the photometric mode of operation when PAI measurements are being performed by the system. Also, the RFID tag reading/writing subsystem 135 provided in the system of FIG. 11B performs the same functions as supported in the system of FIG. 10A.

FIG. 11D describes a formula that can be used to compute the Photo-Activation Index (PAI) of an aspirated tissue sample at any testing interval within the instrument system 200 of FIG. 11B having the photometrically-controlled photo-activation chamber design specified in FIGS. 11E1 and 11E2.

FIG. 11F illustrates the timing of the photo-activation periods relative to the photometric periods of the instrument system 200 of FIG. 11B. As shown, in the illustrative embodiment, the duty cycle of the photo-activation period is about nine (9) times longer than the duty cycle of the photometric period when the Photo-Activation Index (PAI) of the tissue sample is measured and then used to control the photo-activation process of the present invention, to achieve optimal treatment without overdosing and resulting in deleterious effects.

During each photo-activation period, each 635 nm LED and 830 nm LED is driven to achieve low-level photo-active energy exposure upon an aspirated fat tissue sample, having a maximum volumetric power density that does not exceed a predetermined range for aspirated fat tissue (e.g. 5-60 Joules/Volume). During this time, the photo-diodes remain idle and no photometric measurements are made or recorded. Preferably, time duration of each photo-activation period (i.e. interval) is estimated to be about 30 [seconds], followed by a relatively short photometric period, then followed by another photo-activation period, repeating in a cyclical manner in accordance with the photometrically-controlled photo-activation process of the present invention, described in FIGS. 9C1 and 9C2 or 9D1 and 9D2.

During each photometric period, the following operations are automatically performed: (i) driving the RED LEDs indicated in FIGS. 11E1 and 11E2 and measuring and recording intensity response of the tissue sample within the FOV of the respective photo-diode; (ii) driving the IR LEDs indicated in FIGS. 11E1 and 11E2 and measuring and recording intensity response of the tissue sample within the FOV of the respective photo-diode; and (iii) processing the measured intensities of RED and IR signals according to the PAI formula set forth above to compute a Photo-Activation Index (PM) that provides a logarithmic ratio of RED and IR photon affinities in the tissue specimen, in accordance with the principles of the present invention. As with the system of FIG. 10A PAI value is recorded in memory 127, 128, and the microprocessor 125 carries out the photometrically-controlled photo-activation process programmed into system memory. When the optimal PAI has been attained, expectedly taking anywhere between 30 seconds and 5 minutes, depending on the photo-activation state of the aspirated tissue specimen when inserted into the photometrically-controlled photo-activation chamber 101′.

Tissue Photo-Activation Instrument System of the Third Illustrative Embodiment of The Present Invention

FIG. 12A shows a third illustrative embodiment of the tissue authentication and photo-activation instrument system of present invention 300 capable of authenticating and photo-activating a collected fat tissue sample contained in a sealed tissue collection device 20 loaded within a hand-held tissue injector gun 301, and enveloped within its photometrically-controlled photo-activation chamber 101 inside a pod-like housing 302 containing the chamber 101 and RFID tag reading/writing module 135′, and having a flexible cable 304 that establishes an electrically interface with the console unit 305 having a housing 306, controls 307, an LCD touch-screen display panel 308, and display 308B. System 300 is capable of authenticating and photo-activating a collected fat tissue sample contained in the sealed tissue collection and processing device 20 loaded within the photometrically-controlled photo-activation chamber 101 in the pod housing 302.

Prior to performing tissue reinjection operations, the photometrically-controlled photo-activation chamber 101 is installed about a sealed tissue collection and processing device 20 mounted on a hand-held tissue injector gun 301, as shown in FIGS. 12A and 12D. As shown in FIG. 12D, when the device 20 is mounted in the tissue injector gun 301, the plunger 19 of the device 20 is engaged with a rachet-like mechanism 310 that is incrementally advances the plunger 19 into the mounted tissue collection tube 11 upon each manual actuation of a mechanical trigger 311, rotatably mounted in the injector gun housing 312, and engaging the rachet-like mechanism via a cam mechanism. During tissue injection operations, a cannula 21 is mounted onto the distal tip portion of the device 20, as described hereinabove.

During photo-activation treatment, the fat tissue contained in the sealed tissue collection and processing device 20 undergoes automatically-controlled photo-activation treatment by low levels of photo-active light energy having photo-active wavelengths of about 635 and 830 nanometers, emitted from arrays of visible light emitting diodes (LEDs) and/or visible laser diodes (VLDs) surrounding the tissue collection tube 20, while the photo-activation index (PAI) of the fat tissue sample is photometrically-measured between periodically-alternating modes of photo-activation, so as to ensure that the fat tissue sample is optimally photo-activated, and over-activation is avoided.

During photo-activation, the specimen of fat tissue undergoes photo-activation treatment by low level light (LLL) energy having wavelengths of about 635 and 830 nanometers, emitted from arrays of visible light emitting diodes (LEDs) or visible laser diodes (VLDs) surrounding the sealed tissue collection tube 20, while the photo-activation index (PAI) of the fat tissue sample is photometrically-measured between periodically-alternating modes of photo-activation, to ensure that the fat tissue sample is optimally photo-activated, and over-activation is avoided.

As shown in FIG. 12B, the tissue authentication and photo-activation instrument system 300 comprises a number of subcomponents, namely: a plurality of photo-detection (i.e. photo-diode) arrays 110A through 110D, with good response characteristics over the 635 nm and 830 nm regions of the spectrum, and whose analog output signals are provided to a band of pre-amplifier circuits 111, band-pass filters 112, and analog/digital (A/D) converters 113, interfaced with the system bus 114 of the system; a plurality of illumination arrays (e.g. formed from 635 nm and 830 nm LEDs and/or VLDs) 115A through 115D, driven by drive currents supplied by drive circuits 116 which are controlled by digital/analog (D/A) converters 117, interfaced with the system bus 114, as shown; a piezo-electric transducer 118, for generating vibrations that agitate the tissue sample in a tissue collection tube 20 during photo-activation, driven by the output signals from a D/A converter 119 which is interfaced with the system bus 114; reference signal generating circuits 120; a clock 121 for generating clock timing signals used by the system; an AC/DC power adapter 122 interfaced with a power distribution circuit 123, supplied with backup power from a backup battery 124, and supplying electrical power to all electrical power consuming components within the system; a micro-processor 125, interfaced with the system bus 114 and supported by a memory subsystem including SDRAM 126, EPROM 127, FLASH RAM 128, and FLASH drive 129; a communication interface 130 interfaced with the system bus 114, and supporting wireless WIFI communication protocols, Ethernet, USB, Firewire, Serial and other networking protocols; an input/output (I/O) interface 131 for interfacing a LCD touchscreen display panel 308, membrane keypad 308B, LCD display panel 309, and audio transducer 133; and RFID tag reading/writing subsystem 135 interfaced with the system bus 114, including an antenna, for communicating with diverse types of RFID R/W tags 16 applied to the tissue collection and processing devices 20 of the present invention, and reading from and writing to memory onboard these RFID tags 16 during the photometrically-controlled photo-activation process of the present invention.

FIG. 12C shows the hand-held tissue injector gun 301 employed in the tissue authentication and photo-activation instrument system 300 of FIG. 12A, with its photo-activation/photometric pod 302 removed from about the loaded tissue collection device 20.

FIG. 12D shows the hand-held tissue injector gun 310 with its photo-activation/photometric pod 302 installed upon a loaded RFID-tagged tissue collection and processing device 20.

FIG. 12D shows semi-transparent view of the photometrically-controlled photo-activation chamber 101 contained within pod housing 302 and installed on a RFID-tagged tissue collection and processing device 135′ that has been mounted within the pod housing 302. As shown, the RFID tag reading/writing subassembly 135′ is installed within the pod housing 302 proximate to the proximal end of the tissue collection and processing device 20, so that the subsystem is able to read data from and write data to the RFID tag 16 during photo-activation operations, via electromagnetic communication via RF antennas in the RFID tag 16 and RFID reading/writing subsystem 135′ in a manner known in the art.

In all respects, the photometrically-controlled photo-activation chamber 101 shown in FIGS. 12A and 12D is similar to the photometrically-controlled photo-activation chamber 101 shown in FIGS. 10D, and 10G1 and 10G2. However, it is understood that the photometrically-controlled photo-activation chamber 101 employed in the pod 302 can be extended to treat longer length RFID tagged tissue collection and processing devices 20 according to the present invention, as applications may require. Also, the inner diameter of the photometrically-controlled photo-activation chamber 101 can be enlarged or made smaller to adapted to the outer diameter of the RFID tagged tissue collection and processing devices 20. Also, it is understood that the photometrically-controlled photo-activation chamber can be provided with a piezo-electric transducer 118 mounted in direct contact with the side wall portions of the sealed tissue collection and processing device 20, so as to vibrate and uniformly mix during photo-activation operations supported within the photo-activation instrument system of FIG. 12B, and optionally, during the photometric mode of operation when PAI measurements are being performed by the system. Also, the RFID tag reading/writing subsystem 135′ provided in the system 300 of FIG. 12B performs the same functions as supported in the system of FIG. 10A.

FIG. 12E describes a formula that can be used to compute the Photo-Activation Index (PAI) of an aspirated tissue sample at any testing interval within the instrument system 300 of FIG. 12B having the photometrically-controlled photo-activation chamber design specified in FIGS. 12F1 and 12F2.

FIG. 12G illustrates the timing of the photo-activation periods relative to the photometric periods of the instrument system 300 of FIG. 12B. As shown, in the illustrative embodiment, the duty cycle of the photo-activation period is about nine (9) times longer than the duty cycle of the photometric period when the Photo-Activation Index (PAI) of the tissue sample is measured and then used to control the photo-activation process of the present invention, to achieve optimal treatment without overdosing and resulting in deleterious effects.

During each photo-activation period, each 635 nm LED and 830 nm LED is driven to achieve LLE energy exposure upon an aspirated fat tissue sample, having a maximum volumetric power density that does not exceed a predetermined range for aspirated fat tissue (e.g. 5-60 Joules/Volume). During this time, the photo-diodes remain idle and no photometric measurements are made or recorded. Preferably, time duration of each photo-activation period (i.e. interval) is estimated to be about 30 [seconds], followed by a relatively short photometric period, then followed by another photo-activation period, repeating in a cyclical manner in accordance with the photometrically-controlled photo-activation process of the present invention, described in FIGS. 9C1 and 9C2 or 9D1 and 9D2.

During each photometric period, the following operations are automatically performed: (i) driving the RED LEDs indicated in FIGS. 12F1 and 12F2 and measuring and recording intensity response of the tissue sample within the FOV of the respective photo-diode; (ii) driving the IR LEDs indicated in FIGS. 12F1 and 12F2 and measuring and recording intensity response of the tissue sample within the FOV of the respective photo-diode; and (iii) processing the measured intensities of RED and IR signals according to the PAI formula set forth above to compute a Photo-Activation Index (PAI) that provides a logarithmic ratio of RED and IR photon affinities in the tissue specimen, in accordance with the principles of the present invention.

As with the system of FIG. 10A PAI value is recorded in memory 127, 128, 129, and the microprocessor 125 carries out the photometrically-controlled photo-activation process programmed into system memory 127, 128. When the optimal PAI has been attained, expectedly taking anywhere between 30 seconds and 5 minutes, depending on the photo-activation state of the aspirated tissue specimen when inserted into the photometrically-controlled photo-activation chamber 101.

Tissue Photo-Activation Instrument System of the Fourth Illustrative Embodiment of The Present Invention

FIG. 13A is a schematic representation showing a fourth illustrative embodiment of the tissue photo-activation instrument system of present invention 400, capable of photo-activating fat tissue in vivo using a hand-held photo-activation instrument having a hand-supportable housing 401 having integrated hard-key controls 402, and touch-screen display panel 403. This hand-supportable instrument 400 has a photometrically-controlled photo-activation module 405 installed within the distal portion of its hand-supportable housing. With this arrangement, the open end of the module can make contact with in vivo fat tissue, in slow manually-performed scanning motion across the patient's skin, while the patient's underlying tissue undergoes photometrically-controlled photo-activation treatment by low levels of photo-active light energy having photo-active wavelengths of about 635 and 830 nanometers, emitted from an array of light emitting diodes (LEDs) and/or laser diodes (VLDs) mounted within the module, as shown in FIGS. 13A and 13B, and penetrating within the fat tissue beneath the scanned skin. Simultaneously, and transparent to the user and patient, the photo-activation index (PM) of the fat tissue sample is photometrically-measured between periodically-alternating modes of photo-activation, and used to control the dosage of low levels of photo-active energy administered to the patient's tissue, so as to ensure that the fat tissue is optimally photo-activated, and over-activation is avoided. This hand-held photo-activation device 400 is intended for use during post tissue grafting or injection operations to treat the area of injection afterwards to maintain the photo-activation state of the transplanted or grafted tissue, and assure an optimal result. Portable instrument 400 is simple to operate and can be used by the doctor, assistant, or the patient.

As shown in FIG. 13B, the tissue authentication and photo-activation instrument system 400 comprises a number of subcomponents, namely: photo-detection (i.e. photo-diode) arrays 110A and 110B with good response characteristics over the 635 nm and 830 nm regions, and whose analog output signals are provided to a band of pre-amplifier circuits 111 band-pass filters 112, and analog/digital (A/D) converters 113, interfaced with the system bus 114 of the system; illumination arrays (e.g. formed from 635 nm and 830 nm LEDs and/or VLDs) 115A and 115B driven by drive currents supplied by drive circuits 116, which are controlled by digital/analog (D/A) converters 117 interfaced with the system bus 114, as shown; reference signal generating circuits 120; a clock 121 for generating clock timing signals used by the system; a rechargeable battery 412 interfaced with a power distribution circuit 123, supplying electrical power to all electrical power consuming components within the system; a micro-processor 125 interfaced with the system bus 114 and supported by a memory subsystem including SDRAM 126, EPROM 127, FLASH RAM 128, and FLASH drive 129; a communication interface 130 interfaced with the system bus 114, and supporting wireless WIFI communication protocols, Ethernet, USB, Firewire, Serial and other networking protocols; an input/output (I/O) interface 131 for interfacing hard-key control panel 402 and a LCD touchscreen display panel 403, and an audio transducer 133; and RFID tag reading/writing subsystem 135″ interfaced with the system bus 114, including an antenna, for communicating with diverse types of RFID R/W tags 16 applied to a patient-specific RFID card 415, and reading from and writing to memory onboard this RFID card after each photometrically-controlled photo-activation treatment process of the present invention. Such photo-activation state data on the patient's in vivo tissue can be uploaded to the patient's records maintained in the central RDBMS 600, and made accessible by the patient's physician. RFID card 415 can also be provided with an electronic-ink display panel 16B for displaying selected information on the surface of the patient-specific card, and taught in U.S. Pat. No. 7,913,908, supra.

In this illustrative embodiment, the RFID tag reading/writing module 135″ can be installed anywhere within the hand-held housing 401 so that this subsystem 400 is able to read data from and write data to the patient's RFID card 415, or like device, before and after photo-activation treatment operations, via wireless electromagnetic communication using the RF antennas in the RFID card 415 and RFID reading/writing subsystem 135″ in a manner known in the art.

FIG. 13D provides a semi-transparent view of the photometrically-controlled photo-activation module 405 employed hand-supportable unit 400 of FIG. 13A. In all important respects, the photometrically-controlled photo-activation module 405 shown in FIG. 13D is similar in many ways to the photometrically-controlled photo-activation chamber 101 and 101′, except that it operates in a reflection mode, rather than in a transmission mode as does the photo-metrically-controlled photo-activation chambers in the other illustrative embodiments described hereinabove. However, it is understood that the photometrically-controlled photo-activation module 405 employed in the hand-held device 400 can be extended to treat wider areas of in vivo tissue, according to the present invention, as applications may require.

FIG. 13C describes a formula that can be used to compute the Photo-Activation Index (PAI) of an aspirated tissue sample at any testing interval within the instrument system of FIG. 13A having the photometrically-controlled photo-activation chamber design specified in FIG. 13D.

As this “in vivo” tissue treatment instrument system 400 employs reflective-type photo-metric measurement (i.e. nitric-oximetry), the system should be configured to observe a “minimum” absorbance to measure a “pulse static” increase in red light reflectivity in the in vivo tissue being photo-actively treated, because fat tissue with increased levels of oxyheme (or Nitric Oxide which mimics oxyheme) will exhibit an increase in red light reflectivity, whereas tissue with decreased levels of oxyheme (or Nitric Oxide) will exhibit a decrease in red light reflectivity.

FIG. 13E illustrates the timing of the photo-activation periods relative to the photometric periods of the instrument system of FIG. 13A. As shown, in the illustrative embodiment, the duty cycle of the photo-activation period is about nine (9) times longer than the duty cycle of the photometric period when the Photo-Activation Index (PAI) of the tissue sample is measured and then used to control the photo-activation process of the present invention, to achieve optimal treatment without overdosing and resulting in deleterious effects.

During each photo-activation period, each 635 nm LED and 830 nm LED is driven to achieve low level photo-active energy exposure upon an aspirated fat tissue sample including stem cells therein, having a maximum volumetric power density that does not exceed an estimated range for aspirated fat tissue (e.g. 5-50 Joules/cm³). During this time, the photo-diodes remain idle and no photometric measurements are made or recorded. Preferably, time duration of each photo-activation period (i.e. interval) is estimated to be about 30 [seconds], followed by a relatively short photometric period, then followed by another photo-activation period, repeating in a cyclical manner in accordance with the photometrically-controlled photo-activation process of the present invention, described in FIGS. 9C1 and 9C2 or 9D1 and 9D2.

During each photometric period, the following operations are automatically performed: (i) driving the RED LEDs indicated in FIG. 13D and measuring and recording intensity response of the tissue sample within the FOV of the respective photo-diode; (ii) driving the IR LEDs indicated in FIG. 3D and measuring and recording intensity response of the tissue sample within the FOV of the respective photo-diode; and (iii) processing the measured intensities of RED and IR signals according to the PAI formula set forth above to compute a Photo-Activation Index (PM) that provides a logarithmic ratio of RED and IR photon affinities in the tissue specimen, in accordance with the principles of the present invention.

As with other photo-activation systems of the present invention, PAI values are recorded in memory, and the microprocessor 125 carries out the photometrically-controlled photo-activation process programmed into system memory 127, 128. When the optimal PAI has been attained, expectedly taking anywhere between 30 seconds and 5 minutes, over the area of treatment, depending on the photo-activation state of the in vivo tissue being treated by the photometrically-controlled photo-activation instrument 400.

Tissue Photo-Activation Instrument System of the Fifth Illustrative Embodiment of The Present Invention

FIG. 14A is a schematic representation showing a fifth illustrative embodiment of the tissue authentication and photo-activation instrument system of present invention 500, realized in a wireless mobile instrument form-factor 501, capable of authenticating and photo-activating an aspirated fat tissue sample contained in a sealed tissue collection tube 10 inserted within a photometrically-controlled photo-activation chamber 101 mounted within its hand-held housing 503, and being wirelessly connected to a base battery charging and communication station, via an RF-based wireless digital communication link established using Bluetooth® or WIFI® communication protocols. As shown, the base station 503 is also interfaced with a host computer system 515, which is internetworked with the infrastructure of the Internet to reach RDBMS servers 600 and 700, and client machines 800, through an LAN or WAN 520.

As shown in FIG. 14A, the wireless mobile instrument 500 has hard-key controls 504 an LCD touch-screen display panel 505, a battery recharging interface 506, and a RF antenna for communication with the base station 503. The base station 503 includes a battery recharging port 507 into which the recharging interface 506 of system 500 can be inserted during recharging operations. The base station also includes an AC/DC power adapter 122, and a battery recharging circuit 508 for recharging rechargeable battery 509 aboard system 500.

Mobile system 500 is capable of authenticating and photo-activating a collected fat tissue sample contained in a sealed tissue collection device 10 inserted within the photometrically-controlled photo-activation chamber 101 of the hand-held unit 501. During photo-activation, the specimen of fat tissue undergoes photo-activation treatment by low levels of photo-active light energy having photo-active wavelengths of about 635 and 830 nanometers, emitted from arrays of visible light emitting diodes (LEDs) or visible laser diodes (VLDs) surrounding the sealed tissue collection tube 10, while the photo-activation index (PAI) of the fat tissue sample is photometrically-measured between periodically-alternating modes of photo-activation, to ensure that the fat tissue sample is optimally photo-activated, and over-activation is avoided.

As shown in FIG. 14B, the tissue authentication and photo-activation instrument system 500 comprises a number of subcomponents, namely: a plurality of photo-detection (i.e. photo-diode) arrays 110A through 110D with good response characteristics over the 635 nm and 830 nm regions, and whose analog output signals are provided to a band of pre-amplifier circuits 111 band-pass filters 112, and analog/digital (A/D) converters 113, interfaced with the system bus 114 of the system; a plurality of illumination arrays (e.g. formed from 635 nm and 830 nm LEDs and/or VLDs) 115A through 115D, driven by drive currents supplied by drive circuits 116, which are controlled by digital/analog (D/A) converters 117 interfaced with the system bus 114, as shown; a piezo-electric transducer 118 for generating vibrations that agitate the tissue sample in a tissue collection tube 10 during photo-activation, driven by the output signals from a D/A converter 119 which is interfaced with the system bus 114; reference signal generating circuits 120; a clock 121 for generating clock timing signals used by the system; rechargeable battery 509 interfaced with power distribution circuit 123 supplying electrical power to all electrical power consuming components within the system; a micro-processor 125 interfaced with the system bus 114 and supported by a memory subsystem including SDRAM 126, EPROM 127, FLASH RAM 128, and FLASH drive 129; a communication interface 130 interfaced with the system bus 114, and supporting wireless WIFI communication protocols, Ethernet, USB, Firewire,

Serial and other networking protocols; an input/output (I/O) interface 1131 for interfacing hard-key control pad 506, LCD touchscreen display panel 505, and audio transducer 133; and RFID tag reading/writing subsystem 135 interfaced with the system bus 114, including an antenna, for communicating with diverse types of RFID R/W tags 16 applied to the tissue collection and processing devices 10, and reading from and writing to memory onboard these RFID tags 16 during the photometrically-controlled photo-activation process of the present invention.

FIG. 14C provides a perspective view of the hand-held unit 501 embodying the photometrically-controlled photo-activation chamber 101

As shown in FIGS. 14B and 14C, the RFID tag reading/writing subassembly 135 is installed within hand-supportable housing 503, proximate to the proximal end of the chamber 101, so that the subsystem is able to read data from and write data to the RFID tag 16 on the tissue collection and processing device 10 loaded therein during photo-activation operations, via electromagnetic communication via RF antennas in the RFID tag 16 and RFID reading/writing subsystem 135 in a manner known in the art.

Within the chamber, an RFID tagged tissue collection and processing device 10 is inserted, allowing the RFID tag reading/writing subsystem 135 to read from and write to the RFID tag during photometrically-controlled photo-activation operations, described in detail hereinabove. In all important respects, the photometrically-controlled photo-activation chamber 101 shown in FIGS. 14B and 14C is similar to the photometrically-controlled photo-activation chamber 101 shown in FIGS. 10D, and 10G1 and 10G2. However, it is understood that the photometrically-controlled photo-activation chamber 101 employed in the hand-held device 501 can be extended to treat longer length RFID tagged tissue collection and processing devices 10, or be made shorter to treat shorter length RFID tagged tissue collection and processing devices 10, as applications may require. Also, the inner diameter of the photometrically-controlled photo-activation chamber 101 can be enlarged or made smaller to adapted to the outer diameter of the RFID tagged tissue collection and processing devices 10. Also, it is understood that the photometrically-controlled photo-activation chamber can be provided with a piezo-electric transducer 118 mounted in direct contact with the bottom distal cap portion 14 of the sealed tissue collection and processing device 10, as shown in FIGS. 14E1 and 14E2, to vibrate and uniformly mix during photo-activation operations supported within the photo-activation instrument system of FIG. 14A, and optionally, during the photometric mode of operation when PAI measurements are being performed by the system. Also, the RFID tag reading/writing subsystem 135 provided in the system of FIG. 14A performs the same functions as supported in the system of FIG. 10A.

FIG. 14D describes a formula that can be used to compute the Photo-Activation Index (PAI) of an aspirated tissue sample at any testing interval within the instrument system 500 of FIG. 14A having the photometrically-controlled photo-activation chamber design specified in FIGS. 14E1 and 14E2.

FIG. 14F illustrates the timing of the photo-activation periods relative to the photometric periods of the instrument system of FIG. 14A. As shown, in the illustrative embodiment, the duty cycle of the photo-activation period is about nine (9) times longer than the duty cycle of the photometric period when the Photo-Activation Index (PAI) of the tissue sample is measured and then used to control the photo-activation process of the present invention, to achieve optimal treatment without overdosing and resulting in deleterious effects.

During each photo-activation period, each 635 nm LED and 830 nm LED is driven to achieve low level photo-active energy exposure upon an aspirated fat tissue sample, having a maximum volumetric power density that does not exceed a predetermined range for aspirated fat tissue (e.g. 5-50 Joules/cm³). During this time, the photo-diodes remain idle and no photometric measurements are made or recorded. Preferably, time duration of each photo-activation period (i.e. interval) is estimated to be about 30 [seconds], followed by a relatively short photometric period, then followed by another photo-activation period, repeating in a cyclical manner in accordance with the photometrically-controlled photo-activation process of the present invention, described in FIGS. 9C1 and 9C2 or 9D1 and 9D2.

During each photometric period, the following operations are automatically performed: (i) driving the RED LEDs indicated in FIGS. 14E1 and 14E2 and measuring and recording intensity response of the tissue sample within the FOV of the respective photo-diode; (ii) driving the IR LEDs indicated in FIGS. 14E1 and 14E2 and measuring and recording intensity response of the tissue sample within the FOV of the respective photo-diode; and (iii) processing the measured intensities of RED and IR signals according to the PAI formula set forth above to compute a Photo-Activation Index (PM) that provides a logarithmic ratio of RED and IR photon affinities in the tissue specimen, in accordance with the principles of the present invention.

As with the system of FIG. 10A PAI value is recorded in memory, and the microprocessor 125 carries out the photometrically-controlled photo-activation process programmed into system memory. When the optimal PAI has been attained, expectedly taking anywhere between 30 seconds and 5 minutes, depending on the photo-activation state of the aspirated tissue specimen when inserted into the photometrically-controlled photo-activation chamber 101.

Method of Aspirated Tissue Processing According to a First Illustrative Embodiment of The Present Invention

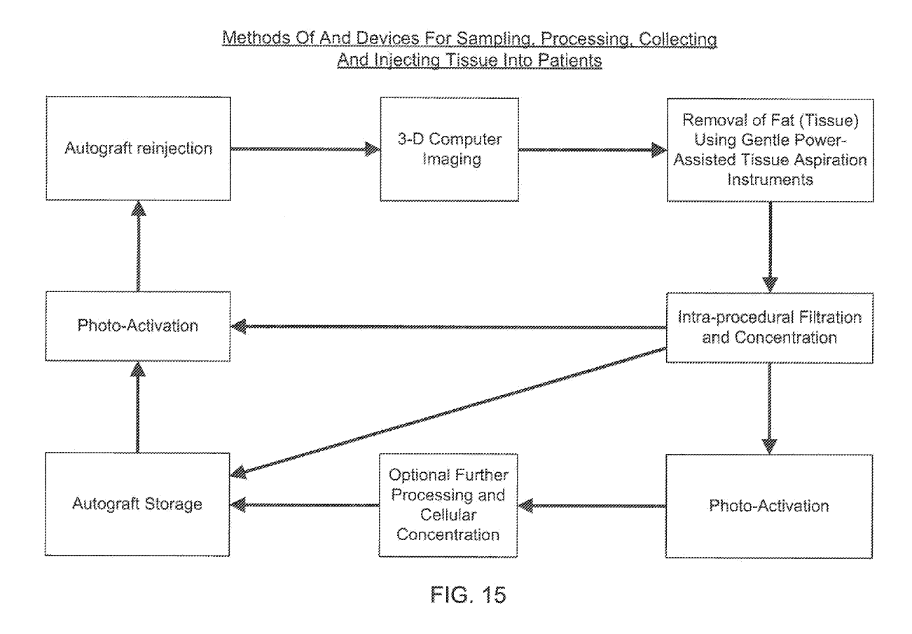

FIG. 15 describes the primary steps involved in carrying out the first method of sampling, collecting, processing and injecting aspirated tissue samples into patients in accordance with the principles of the present invention.

As indicated in FIG. 15, any of the systems 100, 200, 300, and 500 described above can be used to photo-actively treat fat tissue in vitro at various stages, namely: after harvesting from the patient or donor; before immediate reinjection into the patient; before shipping to the tissue bank; before tissue bank growth and differentiation; before shipment of banked tissue to the doctor; and before the doctor injects autograft into the patient.

Also, as indicated in FIG. 15, system 400 described above can be used to photo-actively treat fat tissue in vivo at various stages, namely: immediately after autograft in injection into the patient (i.e. thru intact skin); and during subsequent patient re-visits to the doctor after the injection into the patient.

Method of Aspirated Tissue Processing According to a Second Illustrative Embodiment of The Present Invention

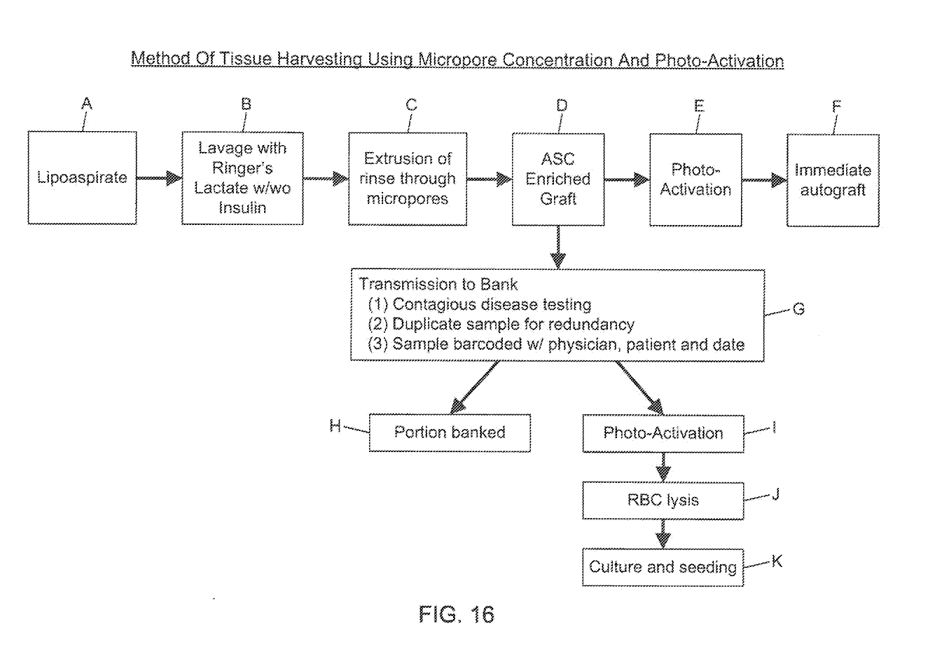

FIG. 16 describes the primary steps involved in carrying out a second method of harvesting, concentrating and photo-activating tissue samples in accordance with the principles of the present invention.

As indicated at Block A in FIG. 16, a lipoaspirate is obtained by harvesting using device 25 or 30 described above, each employing the tissue collection and processing device 10. Device 10 is then extended into device 20 by removing distal cap 14 and adding the micro-pore occluder 13, and plunger 19, as described hereinabove.

At Block B, using device 20, the lipoaspirate is lavaged with Ringers lactate with or without Insulin, to remove blood and oils from the fat tissue sample.

At Block C, the tissue collection and processing device 20 is manually configured and operated to express the rinse fluids out of the micro-pores 12A, 12B and concentrate the tissue cells, to produce an ASC enriched tissue graft at Block D.

At Block E, while still contained in its sealed tissue collection and processing device 10, the ASC enriched graft is then treated with the photo-activation process of the present invention using any one of the in vitro instruments described in detail hereinabove.

Then, at Block F, the photo-activated ASC enriched tissue graft is ready for immediate autograft in various types of treatments. Device 25 can be configured from device 20 as described above to allow the photo-activated autograph to be injected into a desired treatment site on the patient.

Alternatively, the ASC enriched tissue graft at Block D can be transmitted to the tissue (and stem cell) bank as described above, transmitting tissue/stem cell/patent information records from the RIFID tag 16 on the tissue collection and processing device 10, to the central RDBMS 600 on the network 1.

At the tissue and stem cell bank, the following procedures will be performed on the ASC enriched tissue graft: (i) contagious disease testing; duplicate sample preparation for redundancy; and (iii) bar coding of the sealed tissue collection and processing device 10 containing the ASC enriched graft, using at least physician, patient, and date information stored in the RFID tag 16 during the course of history of the collected and processed tissue sample.

At Block H, a redundant portion of the tissue sample can be banked. At Block I, the tissue sample is photo-activated once again with the photo-activation process of the present invention using any one of the in vitro instruments described in detail hereinabove.

At Block J, a red blood cell (RBC) lysis is performed on the tissue sample and the RBC can be recorded in the RFID tag of its tissue collection and processing device.

At Block K, the ASC enriched tissue sample is ready for culturing and seeding in a manner known in the art.

Method of Aspirated Tissue Processing According to a Third Illustrative Embodiment of The Present Invention

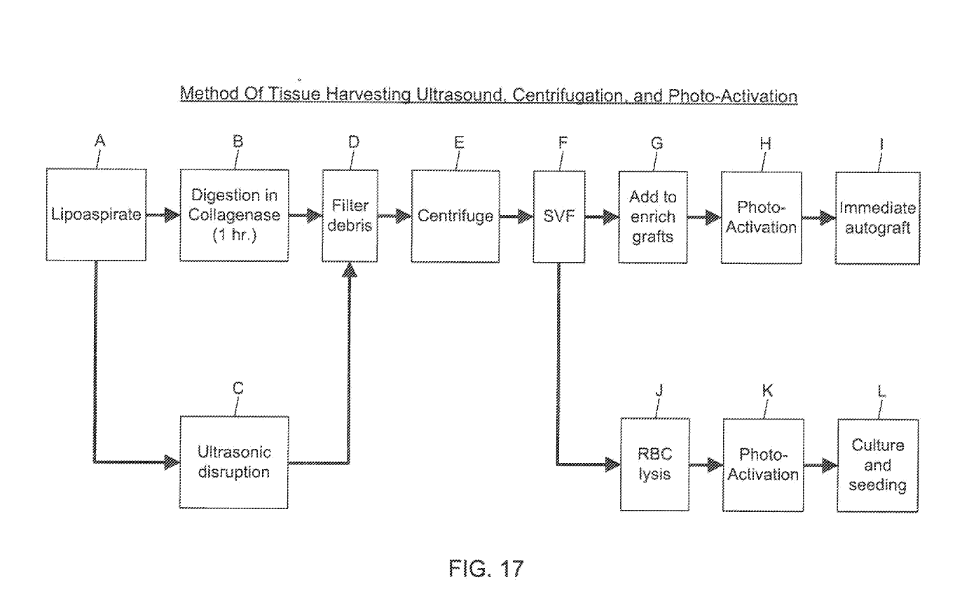

FIG. 17 describes the primary steps involved in carrying out a third alternative method of harvesting, concentrating and photo-activating tissue samples in accordance with the principles of the present invention.

As indicated in FIG. 17, a lipoaspirate (e.g. adipose tissue) sample is harvested using conventional devices.

At Block B, the tissue sample is digested in collagense (for 1 hour).

At Block C, the tissue sample is subject to ultrasonic disruption.

At Block D, the sample is then passed through a debris filter.

At Block E, the sample is placed in a centrifuge to separate the fluids from the solid components.

At Block F, preparation of the stromal vascular fraction (SVF) is derived from the adipose tissue sample.

At Block G, the SVF is added to tissue sample to enrich grafts.

At Block H, the enriched tissue sample is photo-activated with the photo-activation process of the present invention using any one of the in vitro instruments described in detail hereinabove.

At Block I, the photo-activated tissue sample is ready for immediate autograft into the patient.

Alternatively, after Block F, a red blood cell (RBC) lysis can be performed at Block J on the tissue sample and the RBC can be recorded.

At Block K, the tissue sample is photo-activated with the photo-activation process of the present invention using any one of the in vitro instruments described in detail hereinabove.

At Block L, the photo-activated tissue sample is ready for culturing and seeding in a manner known in the art.

Modifications That Come to Mind

The above-described process has been provided as an illustrative example of how photometrically-controlled photo-activation of fat tissue can be practices in both in vitro and in vivo environments.

In the illustrative embodiments, the photo-activation wavelengths 635 nm and 630 nm were selected because of their known photo-active properties to stimulate chromophores in cells including stem cells in aspirated fat tissue (i.e. once such tissue has been lavaged to remove blood and oils from the specimen). However, it is understood that deviation from such specified wavelengths is expected to occur when practicing the present invention, especially as more is learned about the effects of photo-active light energy on the bio-energy of cells and cellular components in aspirated fat tissue.

Also, in alternative embodiment, different wavelengths can be transmitted into aspirated tissue samples, and into stem cells therein, to promote cell differentiation. Pulsed modes of low levels of photo-active energy transmitted through aspirated tissue samples, including stem cells therein, can be used, along with other (e.g. high-speed optical modulation) techniques, to generate spectral harmonics that photo-activate cellular organelles, promote cell growth and or differentiation, and increase the bio-energy states of living tissue.

Based on the principles of the present invention, photometrically-controlled photo-activation systems can be designed and manufactured which are capable of simultaneously photo-activating multiple sealed tissue collected and processing devices 10, to increase the rate of tissue and stem cell processing.

Variations and modifications to this process will readily occur to those skilled in the art having the benefit of the present disclosure. All such modifications and variations are deemed to be within the scope of the accompanying Claims.

1. A fat tissue re-injection system comprising:
a hand-held tissue injector gun for mounting a tissue collection and processing device and supporting a cannula for injecting fat tissue from said tissue collection and processing device into a patient, during tissue re-injection operations; and
a photometrically-controlled photo-activation chamber for installation about said tissue collection and processing device, mounted on said hand-held tissue injector gun;
wherein, prior to performing fat tissue reinjection operations, fat tissue including stem cells therein contained in said sealed tissue collection and processing device is exposed to automatically-controlled photo-activation treatment within said photometrically-controlled photo-activation chamber, using low levels of photo-active light energy having photo-active wavelengths emitted from arrays of visible light emitting diodes (LEDs) and/or visible laser diodes (VLDs) surrounding the tissue collection tube; and
wherein the photo-activation index (PAI) of said fat tissue sample is photometrically-measured between periodically-alternating modes of photo-activation, so as to ensure that said fat tissue sample including said stem cells are optimally photo-activated, and over-activation is avoided.

2. A fat tissue injection gun comprising:
a hand-held housing having a manually actuable trigger mechanism; and
a support device extending from said hand-held housing, for supporting a tissue collection tube having (i) a distal end opening for attachment of a cannula, (ii) a proximal end opening leading into an interior volume in communication with said distal end opening, for containing a fat tissue sample, and (iii) a plunger and piston assembly inserted into said interior volume and operable connecting with said trigger mechanism;
wherein as said trigger mechanism is manually actuated, said plunger and piston assembly is pushed into said interior volume, causing the fat tissue sample to be ejected from said distal end opening, out of said cannula and into a patient, during fat tissue injection operations.
